(19) United States
(12) Patent Application Publication
(10) Pub. No.: US 2014/0350064 A1
(43) Pub. Date: Nov. 27, 2014

(54) PHARMACEUTICAL COMPOSITION COMPRISING AN AMPK ACTIVATOR AND A SEROTONERGIC AGENT AND METHODS OF USE THEREOF

(71) Applicant: ALS MOUNTAIN LLC, Forest Hills, NY (US)

(72) Inventor: CHIEN-HUNG CHEN, Forest Hills, NY (US)

(21) Appl. No.: 14/212,734

(22) Filed: Mar. 14, 2014

Related U.S. Application Data
(60) Provisional application No. 61/793,407, filed on Mar. 15, 2013.

(51) Int. Cl. A61K 31/4045; A61K 31/137; A61K 31/155

(52) U.S. Cl. CPC A61K 31/4045; A61K 31/155; A61K 31/137
USPC 514/415

(57) ABSTRACT

The present invention is based on the unexpected discovery that a combination of certain known drugs exhibits synergistic effects in treating metabolic syndrome and various other diseases. In particular, the invention comprises a pharmaceutical composition comprising: (1) a therapeutically effective quantity of a first agent that is an AMPK activator; and (2) a therapeutically effective quantity of a second agent that possesses or maintains serotonin activity. A preferred composition comprises metformin hydrochloride and melatonin. The invention further comprises methods for the use of these compositions for the treatment of metabolic syndrome, hyperproliferative diseases including cancer, and other diseases and conditions.

### CROSS-REFERENCES TO RELATED APPLICATION

This application claims the benefit of United States Provisional Patent Application Ser. No. 61/793,407 by Chien-Hung Chen, entitled “Pharmaceutical Composition Comprising an AMPK Activator and a Serotonergic Agent and Methods of Use Thereof, filed on Mar. 15, 2013, the contents of which are hereby incorporated in their entirety by this reference.

### FIELD OF THE INVENTION

This invention is directed to pharmaceutical compositions comprising a 5′-adenosine-monophosphate-activated kinase (AMPK) activator and a serotonergic agent and the use of these pharmaceutical compositions for a number of diseases and conditions.

### BACKGROUND OF THE INVENTION

Metabolic syndrome is characterized by a group of metabolic risk factors, including abdominal obesity, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels). Metabolic syndrome has become increasingly common in the United States. It is estimated that over 50 million Americans have this disorder. There is a need to develop novel drugs to effectively treat this disorder.

According to the World Health Organization, about five million people die from cancer every year. Drug treatment is one of the three major therapies for cancer. At present, drugs are used to treat cancers by the following mechanisms: interfering with or inhibiting cell division, regulating cell generation cycle, promoting tumor cell apoptosis, inhibiting angiogenesis, inhibiting oncogene activity, promoting tumor-suppressing gene activity, acting as tumor antigens, inhibiting telomerase activities, and interfering with information transfer of tumor cells.

In view of the high mortality rates associated with abnormal proliferative diseases including cancer, there exists a need for an effective treatment for these diseases.

Acquired immunodeficiency syndrome (AIDS), a consequence of infection with the HIV-1 retrovirus, affects over 30 million people worldwide. AIDS is characterized by a number of otherwise very rare opportunistic infections such as Kaposi's sarcoma, caused by the Kaposi's sarcoma-associated herpes virus, Pneumocystis jirovecii pneumonia, and other malignancies and infectious diseases. Patients with AIDS also suffer from severe weight loss, night sweats, swollen lymph nodes, and other consequences of a compromised immune system. In AIDS, CD4⁺ T cells are attacked by the virus and greatly reduced in number. Although treatments for AIDS do exist, including treatment with a “cocktail” of three drugs belonging to at least two classes of antiretroviral drugs, such as, for example, two nucleoside analogue reverse transcriptase inhibitors plus either a protease inhibitor or a non-nucleoside reverse transcriptase inhibitor. Although this approach has proved reasonably successful in inhibiting the growth of HIV-1 and preventing the occurrence of opportunistic infections and other symptoms of AIDS, it is not a cure and the effectiveness of drug therapy can be limited by drug resistance, drug toxicity, and possible patient non-compliance. Therefore, there is a need for an improved therapy for AIDS.

### BRIEF SUMMARY OF THE INVENTION

The present invention provides pharmaceutical compositions and methods that are suitable for treating a number of diseases and conditions, including: metabolic syndrome and diseases and conditions associated with metabolic syndrome, including diabetes, obesity, and hypertension; hyperproliferative diseases and conditions including cancer; AIDS; Parkinson's disease; polycystic ovarian syndrome, Alzheimer's disease; osteoporosis; sleep apnea; erectile dysfunction; McArdle disease; and carbohydrate metabolism disorders, as well as being useful for treating aging or fatigue.

This invention is based on the unexpected discovery that a combination of certain known drugs exhibits synergistic effects in treating metabolic syndrome and various other diseases.

One aspect of the invention is a pharmaceutical composition comprising:

(1) a therapeutically effective quantity of a first agent that is an AMPK activator; and

(2) a therapeutically effective quantity of a second agent that possesses or maintains serotonin activity.

The AMPK activator can be an AMPK activator selected from the group consisting of (1) metformin; (2) phenformin; (3) buformin; (4) AICAR; (5) a thienopyridone; (6) resveratrol; (7) nootkatone; (8) thiazole; (9) adiponectin; (10) 2-deoxyglucose; (11) AAPDs; (12) adiponectin variant polypeptides; (13) catechins; (14) trans-10, cis-12 conjugated linoleic acid; (15) a corydaline-related compound selected from the group consisting of corydaline, corlumidin, (+)-corlumidin, corypalmine, 14R-(+)-corypalmine, tetrahydropalmatine, 14R-(+)-tetrahydropalmatine, 14R,13S-(+)-corydaline, bicuculline, d-(+)-bicuculline, egenine, and +-egenine; (16) a dithiolethione; (17) an inhibitor or antagonist of DNA-dependent protein kinase catalytic subunit (DNA-PKcs); (18) a small interfering RNA (siRNA) that can inhibit the expression and/or translation of DNA-PKcs; (19) a fibrate selected from the group consisting of bezafibrate, ciprofibrate, fenofibrate, clofibrate, and gemfibrozil; (20) GW2974 (N4-(1-benzyl-1H-indazol-5-yl)-N6,N6-dimethyl-pyrido-[3,4-d]-pyrimidine-4,6-diamine); (21) honokiol; (22) leptin; (23) LKB1 (serinethreonine kinase 11); (24) obovatol (4′,5-diallyl-2,3-dihydroxybiphenyl ether); (25) a thiazolidinedione selected from the group consisting of pioglitazone and related thiazolidinediones, including rosiglitazone and rosiglitazone maleate; (26) a variant adiponectin peptide having one or more mutations at amino acid positions 109-229 of wild-type adiponectin and having at least threefold increased solubility when compared to wild-type adiponectin; (27) a butyrate compound selected from a butyrate salt and a butyrate ester; and (28) a quinoxalinedione derivative; and the salts, solvates, analogues, congeners, bioisosteres, hydrolysis products, metabolites, precursors, and prodrugs thereof. Typically, the first agent is selected from the group consisting of metformin, phenformin, buformin, AICAR, thienopyridones, resveratrol, nootkatone, thiazole, adiponectin, thiazolidinediones, rosiglitazone, pioglitazone, dithiolethiones, and the salts, solvates, analogues, congeners, bioisosteres, hydrolysis products, metabolites, precursors, and prodrugs thereof. A particularly preferred AMPK activator is metformin or a salt thereof, such as metformin hydrochloride.

The second agent can be serotonin or a serotonin metabolite, such as a compound selected from the group consisting of serotonin sulfate, serotonin creatinine sulfate complex, serotonin hydrochloride, melatonin, 5-hydroyxindoleacetic acid, a salt of 5-hydroxyindoleacetic acid, melatonin creatinine sulfate complex, and 5-hydroxyindoleacetic acid creatinine sulfate complex. A particularly preferred second agent is melatonin.

Alternatively, the second agent can be a serotonergic compound such as a serotonergic compound selected from the group consisting of: (1) serotonin transport inhibitors; (2) serotonin receptor 2C modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin agonists; (11) serotonin receptor 1A antagonists; and (12) serotonin metabolites.

The composition can further comprise a pharmaceutically acceptable carrier. The first or second agents can be associated with a carrier substance or carrier substances to facilitate the transport of the first agent or the second agent to an intended site of action of the first agent or the second agent.

Another aspect of the present invention is a method of treating a disease or condition comprising the step of administering a therapeutically effective quantity of a pharmaceutical composition according to the present invention as described above to a subject that has the disease or condition or that is at risk of developing the disease or condition, in order to treat or prevent the occurrence of the disease or condition, wherein the disease or condition is selected from the group consisting of metabolic syndrome, diabetes, obesity, hypertension, cancer, AIDS, Parkinson's disease, polycystic ovarian syndrome, Alzheimer's disease, osteoporosis, sleep apnea, erectile dysfunction, McArdle disease, and a carbohydrate metabolism disorder. Typically, the disease or condition is selected from the group consisting of metabolic syndrome, diabetes, obesity, and hypertension. In another alternative, the disease or condition is cancer. In still another alternative, the disease or condition is selected from the group consisting of Parkinson's disease, polycystic ovarian syndrome, Alzheimer's disease, osteoporosis, sleep apnea, erectile dysfunction, McArdle disease, and a carbohydrate metabolism disorder. The pharmaceutical composition can be administered orally or parenterally.

### BRIEF DESCRIPTION OF THE DRAWINGS

The following invention will become better understood with reference to the specification, appended claims, and accompanying drawings, where:

FIG. 1 is a graph showing the increase of body weight for rats treated with either metformin plus melatonin or sibutramine as compared with untreated rats based on data from the Example.

FIG. 2 is a graph showing the average food intake for rats treated with either metformin plus melatonin or sibutramine as compared with untreated rats based on data from the Example.

FIG. 3 is a graph showing the average total fat mass for rats treated with either metformin plus melatonin or sibutramine as compared with untreated rats based on data from the Example.

### DETAILED DESCRIPTION OF THE INVENTION

This invention is based on the unexpected discovery that a combination of certain known drugs exhibits synergistic effects in treating metabolic syndrome and various other diseases. In addition to metabolic syndrome and diseases and conditions associated with metabolic syndrome, the combination of these known drugs can be used to treat hyperproliferative disease (including cancer), AIDS, Parkinson's disease, polycystic ovarian syndrome, Alzheimer's disease, osteoporosis, sleep apnea, erectile dysfunction, McArdle disease, and carbohydrate metabolism disorders. The combination of these known drugs can also be used to treat aging or fatigue. The combination of these known drugs can also be used to treat a disease or condition such as: (1) cardiac dysrhythmias; (2) endometriosis, uterine fibroid (uterine leiomyomata) menorrhagia, cervical erosion, cervical polyp, and related conditions; and (3) defects or disorders of intervertebral discs.

In one aspect, the invention comprises a pharmaceutical composition comprising:

(1) a therapeutically effective quantity of a first agent that is an activator of 5′-adenosine-monophosphate-activated kinase (AMPK); and

(2) a therapeutically effective quantity of a second agent that possesses or maintains serotonin activity.

AMPK activators include, but are not limited to: (1) metformin; (2) phenformin; (3) buformin; (4) AICAR; (5) thienopyridones; (6) resveratrol; (7) nootkatone; (8) thiazole; (9) adiponectin; (10) 2-deoxyglucose; (11) AAPDs (atypical antipsychotic drugs, including olanzapine, quetiapine, and risperidone); (12) adiponectin variant polypeptides such as AdipoR3v1 polypeptide, AdipoRe polypeptide, and AdipoR2vs polypeptide, disclosed in U.S. Pat. No. 7,435,808 to Wu et al., incorporated herein by this reference; (13) catechins, including catechin, gallocatechin, catechin gallate, gallocatechin gallate, epicatechin, epigallocatechin, epicatechin gallate and epigallocatechin gallate, disclosed in United States Patent Application Publication No. 20070004650 by Shimotoyodome et al., incorporated herein by this reference; (14) trans-10, cis-12 conjugated linoleic acid; (15) corydaline and related compounds, including corlumidin, (+)-corlumidin, corypalmine, 14R-(+)-corypalmine, tetrahydropalmatine, 14R-(+)-tetrahydropalmatine, 14R,13S-(+)-corydaline, bicuculline, d-(+)-bicuculline, egenine, and +-egenine, disclosed in United States Patent Application Publication No. 2009/0042810 by Chung and United States Patent Application Publication No. 2009/048246 by Lin et al., both of which are incorporated herein by this reference; (16) dithiolethiones, including oltipraz and 5-(4-methoxyphenyl)-3H-1,2-dithiole-3-thione; (17) inhibitors or antagonists of DNA-dependent protein kinase catalytic subunit (DNA-PKcs), disclosed in United States Patent Application Publication No. 2010/0130597 by Chung et al., incorporated herein by this reference; (18) small interfering RNAs (siRNAs) that can inhibit the expression and/or translation of DNA-PKcs, disclosed in United States Patent Application Publication No. 2010/0130597 by Chung et al., incorporated herein by this reference; (19) fibrates, including bezafibrate, ciprofibrate, fenofibrate, clofibrate, and gemfibrozil; (20) GW2974 (N4-(1-benzyl-1H-indazol-5-yl)-N6,N6-dimethyl-pyrido-[3,4-d]-pyrimidine-4,6-diamine); (21) honokiol; (22) leptin; (23) LKB1 (serinethreonine kinase 11); (24) obovatol (4′,5-diallyl-2,3-dihydroxybiphenyl ether); (25) pioglitazone and related thiazolidinediones, including rosiglitazone and rosiglitazone maleate; (26) Y122SI125E and additional muteins of adiponectins, disclosed in U.S. Pat. No. 7,678,886 to Zalevsky et al., incorporated herein by this reference, such as a variant adiponectin peptide with the formula: V(109)-V(110)-V(111)-F(112)-F(113-121)-V(122)-F(123)-V(124)-V(125)-F(126-127)-V(128)-F(129-134)-V(135)-F(136-151)-V(152)-F(153-163)-F-(164)-F(165-181)-V(182)-F(183)-V(184)-F(185-206)-V(207)-F(208-220)-F(221)-F(222-223)-V(224)-V(225)-F(226)-V(227)-F(228)-V(229), wherein V(109) is selected from the group consisting of: the wild-type amino acid V; any of variant amino acids D, E, H, K, N, Q, and R; and, a deletion of V109; V(110) is selected from the group consisting of: the wild-type amino acid V; any of variant amino acids D, E, H, K, N, Q, R, and S; and, a deletion of V110; V(111) is selected from the group consisting of: the wild-type amino acids Y and H; any of variant amino acids D, E, N, R, and S; and, a deletion of 111; F(112) is selected from the group consisting of the wild-type amino acids R and C, and, a deletion of 112; F(113-121) is selected from the group consisting of: the wild-type amino acid sequence SAFSVGLET (SEQ ID NO: 1); and, a deletion of any of S113, A114, F115, S116, V117, G118, L119, E120, and T121; V(122) is selected from the group consisting of: the wild-type amino acid Y; any of variant amino acids D, E, H, N, R, and S; and, a deletion of Y122; F(123) is selected from the group consisting of: the wild-type amino acid sequence V and a deletion of V123; V(124) is selected from the group consisting of: the wild-type amino acid T; any of variant amino acids D, E, K, N, and R; and, a deletion of T124; V(125) is selected from the group consisting of: the wild-type amino acid I; any of variant amino acids D, E, H, K N, Q, R, S, and T; and, a deletion of 1125; F(126-127) comprises the wild-type amino acid sequence PN; V(128) is selected from the group consisting of: the wild-type amino acid M; and any of variant amino acids A, D, E, H, K, N, Q, R, S, and T; F(129-134) comprises the wild-type amino acid sequence PIRFTK (SEQ ID NO: 2); V(135) is selected from the group consisting of: the wild-type amino acid I; and, any of variant amino acids D, E, H, K, N, Q and R; F(136-151) comprises the wild-type amino acid sequence FYNQQNHYDGSTGKFH (SEQ ID NO: 3); V(152) is selected from the group consisting of: the wild-type amino acid C; and, any of variant amino acids A, F, L, N, S, T and V; F(153-163) comprises the wild-type amino acid sequence NIPGLYYFAYH (SEQ ID NO: 4); F(164) is selected from the group consisting of the wild-type amino acid I and T; F(165-181) comprises the wild-type amino acid sequence TVYMKDVKVSLFKKDKA (SEQ ID NO: 5); V(182) is selected from the group consisting of: the wild-type amino acid M; and, any of variant amino acids A, D, E, K, N, Q, R, S, and T; F(183) comprises the wild-type amino acid L; V(184) is selected from the group consisting of: the wild-type amino acid F; and, any of variant amino acids D, H, K, N and R; F(185-206) comprises the wild-type amino acid sequence TYDQYQENNVDQASGSVLLHLE (SEQ ID NO: 6); V(207) is selected from the group consisting of: the wild-type amino acid V; and, any of variant amino acids D, E, H, K, N, Q, R, and S; F(208-220) comprises the wild-type amino acid sequence GDQVWLQVYGEGE (SEQ ID NO: 7); F(221) is selected from the group consisting of the wild-type amino acids R and S; F(222-223) comprises the wild-type amino acid sequence NG; V(224) is selected from the group consisting of: the wild-type amino acid L; and, any of variant amino acids D, E, H, K, N, Q, R and S; V(225) is selected from the group consisting of: the wild-type amino acid Y; and, any of variant amino acids D, E, H, K, N, Q, R and S; F(226) comprises the wild-type amino acid A; V(227) is selected from the group consisting of: the wild-type amino acid D; and, any of variant amino acids H, K and R; F(228) comprises the wild-type amino acid N; or V(229) is selected from the group consisting of: the wild-type amino acid D; and, any of variant amino acids H, K and R, the variant adiponectin having at least threefold increased solubility when compared to wild-type adiponectin; (27) butyrate and butyrate analogs as disclosed in United States Patent Application Publication No. 20110077300 by Ye et al., incorporated herein by this reference, including, but not limited, to butyrate salts, including sodium butyrate, butyl butyrate, n-pentyl butyrate, isobutyl butyrate, α-methylbenzyl butyrate, hexyl butyrate, phenethyl butyrate, methyl butyrate, ethyl butyrate, 2-hydroxy-3-methylbutanoic acid, trimethylbutyrin, a triglyceride with at least one butyrate moiety attached to the glycerol backbone of the triglyceride, preferably two butyrate moieties attached to the glycerol backbone of the triglyceride, wherein the triglyceride also comprises at least one long-chain fatty acid attached to the glycerol backbone of the triglyceride, wherein the long-chain fatty acid is a saturated fatty acid or an unsaturated fatty acid, and in which a preferred long-chain fatty acid is oleate; (28) quinoxalinedione derivatives as described in United States Patent Application Publication No. 20110130404 by Cravo et al., incorporated herein by this reference; (29) thienopyridone derivatives as described in United States Patent Application Publication No. 20110034505 by Cravo et al., incorporated herein by this reference; and (30) thienopyridone derivatives as described in United States Patent Application Publication No. 20110006001 by Cravo et al., incorporated herein by this reference; and the salts, solvates, analogues, congeners, bioisosteres, hydrolysis products, metabolites, precursors, and prodrugs thereof.

Typically, the first agent is selected from the group consisting of metformin, phenformin, buformin, AICAR, thienopyridones, resveratrol, nootkatone, thiazole, adiponectin, thiazolidinediones, rosiglitazone, pioglitazone, dithiolethiones, and the salts, solvates, analogues, congeners, bioisosteres, hydrolysis products, metabolites, precursors, and prodrugs thereof.

Preferably, the first agent is metformin or a salt thereof, such as metformin hydrochloride.

In one alternative, the second agent is serotonin or a serotonin metabolite. The second agent can be, but is not limited to, a compound selected from the group consisting of serotonin sulfate, serotonin creatinine sulfate complex, serotonin hydrochloride, melatonin, 5-hydroyxindoleacetic acid, a salt of 5-hydroxyindoleacetic acid, melatonin creatinine sulfate complex, and 5-hydroxyindoleacetic acid creatinine sulfate complex. Typically, in this alternative, the second agent is a compound selected from the group consisting of melatonin, 5-hydroxyindoleacetic acid, and a salt of 5-hydroxyindoleacetic acid. Preferably, in this alternative, the second agent is melatonin.

In another alternative, the second agent is a serotonergic compound. The serotonergic compound can be, but is not limited to, a serotonergic compound selected from the group consisting of: (1) serotonin transport inhibitors; (2) serotonin receptor 2C modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin agonists; (11) serotonin receptor 1A antagonists; and (12) serotonin metabolites. These categories are not exclusive, and many active serotonergic compounds suitable for inclusion in compositions of the present invention as the third agent can be considered to be in more than one of these categories; for example, such compounds can specifically interact with more than one class of serotonin receptor or more than one subclass of serotonin receptor within a single class.

Serotonin transport inhibitors include paroxetine, fluoxetine, fenfluramine, fluvoxamine, sertraline, imipramine, and compounds disclosed in PCT Patent Application Publication No. WO 03/00663, including phenyl-substituted piperazinylpyrimidines.

Serotonin receptor 2C modulators include BVT933, DPCA37215, IK264, (6-methyl-1,2,3,4,5,6-hexahydro-azepino[4,5-b]indole), WAY161503 (8,9-dichloro-2,3,4,4a-tetrahydro-1H-pyrazino[1,2-a]quinoxalin-5(6H)-one hydrochloride), R-1065, YM348 ((2S)-1-(7-ethyl-1H-furo[2,3-g]indazol-1-yl)propan-2-amine), and compounds disclosed in U.S. Pat. No. 3,914,250 and in PCT Patent Application Publication Nos. WO 01/66548, WO 02/10169, WO 02/36596, WO 02/40456, and WO 02/40457, WO 02/44152, WO 02/48124, WO 02/51844, and WO 03/033479, including 1,4-diazepino[6,5,4-jk]carbazoles, aza-indolyl derivatives, piperazine derivatives, cycloalkenyl[b][1,4]diazepino[6,7,1-hi]indoles and derivatives thereof, piperazinylpyrazine compounds, indoline derivatives, piperazine derivatives, and indole derivatives.

Serotonin reuptake inhibitors include arylpyrrolidine compounds, phenylpiperazine compounds, benzylpiperidine compounds, piperidine compounds, tricyclic gamma-carbolines, duloxetine compounds, pyrazinoquinoxaline compounds, pyridoindole compounds, piperidylindole compounds, milnacipran, citalopram, sertraline metabolite desmethylsertraline, norfluoxetine, citalopram metabolite desmethylcitalopram, escitalopram, d,l-fenfluramine, femoxetine, ifoxetine, cyanodothiepin, litoxetine, dapoxetine, nefazodone, cericlamine, trazodone, mirtazapine, fluoxetine, fluvoxamine, indalpine, indeloxazine, paroxetine, sertraline, sibutramine, zimeldine, trazodone hydrochloride, dexfenfluramine, and compounds disclosed in U.S. Pat. No. 6,365,633, PCT Patent Application Publication No. WO 01/27060, and PCT Patent Application Publicatio No. WO 01/162341, including (+)-N-[1-[1-(4-chlorophenyl)cyclobutyl]-3-methylbutyl}-N-methylamine, (−)-N-{1-[1-(4-chlorophenyl)cyclobutyl-3-methylbutyl}-N-methylamine, (+)-1-[1-(4-chlorophenyl)cyclobutyl]-3-methylbutylamine, (−)-1-[1-(4-chlorophenyl)cyclobutyl]-3-methylbutylamine, (+)-N-{1-[1-(4-chlorophenyl)cyclobutyl]-3-methylbutyl}-N,N-dimethylamine, and (−)-N-{1-[1-(4-chlorophenyl)cyclobutyl]-3-methylbutyl}-N, -dimethylamine.

Serotonin and norepinephrine reuptake inhibitors include venlafaxine, venlafaxine metabolite O-desmethylvenlafaxine, clomipramine, and clomipramine metabolite desmethylclomipramine.

Serotonin dopamine antagonists include olanzapine and ziprasidone.

Monoamine reuptake inhibitors include amides.

Pyridazinone aldose reductase inhibitors include pyridazinone compounds.

Stimulants of serotonin receptors include ergoloid mesylate and pergolide mesylate.

Stimulants of serotonin synthesis include vitamin B1, vitamin B3, vitamin B6, biotin, S-adenosylmethionine, folic acid, folinic acid, derivatives of folic acid and folinic acid, ascorbic acid, magnesium, coenzyme Q10, and piracetam.

Serotonin agonists include fenfluramine and buspirone (a partial agonist for serotonin receptor 1A).

Serotonin receptor 1A antagonists include alprenolol, asenapine, BMY 7378 (8-(2-[4-(2-methoxyphenyl)-1-piperazinyl]ethyl)-8-azaspiro[4.5]decane-7,9-dione), cyanopindolol, iodocyanopindolol, lezcotozan, methiothepin, NAN-190 (1-(2-methoxyphenyl)-4-(4-phthalimidobutyl)piperazine), oxprenolol, pindolol, propranolol, robalzotan, S15535 (1-(2,3-dihydro-1,4-benzodioxin-8-yl)-4-(2,3-dihydro-1H-inden-2-yl)piperazine), spiperone, TFMPP, UH-301 ((S)-5-fluoro-8-hydroxy-2-(dipropylamino)tetralin), WAY-100,135 ((S)—N-tert-butyl-3-(4-(2-methoxyphenyl)-piperazin-1-yl)-2-phenylpropanamide), WAY-100,635 (N-[2-[4-(2-methoxyphenyl)-1-piperazinyl]ethyl]-N-(2-pyridyl)cyclohexanecarboxamide), and mefway.

Serotonin metabolites include, but are not limited to, 5-hydroxytryptophan, 5-methoxytryptamine, melatonin, or 5-HIAA (5-hydroxyindoleacetic acid). Preferably, the serotonin metabolite is present in the form of a creatinine sulfate complex, so that particularly preferred serotonin metabolites, in the form of a creatinine sulfate complex, include, but are not limited to, 5-hydroxytryptophan creatinine sulfate complex, 5-methoxytryptamine creatinine sulfate complex, melatonin creatinine sulfate complex, and 5-HIAA (5-hydroxyindoleacetic acid) creatinine sulfate complex. When the serotonin metabolite is included in the composition described above, it can be substantially free of impurities. For example, the serotonin metabolite can have a purity of at least about 80% (e.g., at least about 85%, at least about 90%, at least about 95%, or at least about 99%).

Many other serotonergic compounds and derivatives and metabolites thereof are known in the art and are included within the scope of the present application. Such serotonergic compounds and derivatives and metabolites thereof include: (1) serotonergic aminoalkylbenzadioxanes, such as those disclosed in U.S. Pat. No. 5,200,410; (2) serotonergic aminotetrahydrobenzindoles, such as those disclosed in U.S. Pat. No. 5,070,102; (3) serotonergic aminothiopyrans, such as those disclosed in U.S. Pat. No. 5,200,410; (4) serotonergic indolamines, such as those disclosed in U.S. Pat. No. 5,200,410; (5) serotonergic indolylalkylpiperidines, such as those disclosed in U.S. Pat. No. 5,200,410; (6) serotonergic monoamine oxidase inhibitors; (7) serotonergic tricyclic antidepressants; (7) serotonergic acetamide or carbamide derivatives, such as those disclosed in U.S. Pat. No. 6,756,393; (8) serotonergic 1-oxa-3,8-diaza-spiro[4.5]decan-2-one compounds such as those disclosed in U.S. Pat. No. 6,911,452; (9) serotonergic N-substituted piperidine derivatives, such as those disclosed in United States Patent Application Publication No. 20040106600; (10) serotonergic 2-pyrimidinyl-1-piperazines, such as those disclosed in U.S. Pat. No. 4,988,700; (11) serotonergic aryl-1-piperazines, such as those disclosed in U.S. Pat. No. 4,988,700; (12) serotonergic L-tryptophan derivatives and peptidyl derivatives of L-tryptophan, such as those disclosed in U.S. Pat. No. 6,579,899; (13) serotonin antagonists, such as those disclosed in United States Patent Application Publication No. 20010008896; and (14) serotonergic substituted dihydroergoline compounds, such as those disclosed in U.S. Pat. No. 4,798,834. Still other compounds are known in the art. Moreover, because of the multiplicity of classes and subclasses of serotonin receptors, some compounds may act as an agonist or partial agonist at one class or subclass of serotonin receptor, such as serotonin receptor 1A or 2A, and yet may act as an antagonist or inverse agonist at another class or subclass of serotonin receptor, such as serotonin receptor 2B, serotonin receptor 2C, serotonin receptor 6, or serotonin receptor 7.

Accordingly, suitable serotonergic compounds according to the present invention include, but are not limited to: (1) paroxetine; (2) fluoxetine; (3) fenfluramine; (4) fluvoxamine; (5) sertraline; (6) imipramine; (7) BVT933; (8) DPCA37215; (9) IK264; (10) PNU22394 (6-methyl-1,2,3,4,5,6-hexahydro-azepino[4,5-b]indole); (11) WAY161503 (8,9-dichloro-2,3,4,4a-tetrahydro-1H-pyrazino[1,2-a]quinoxalin-5(6H)-one hydrochloride); (12) R-1065; (13) YM348 ((2S)-1-(7-ethyl-1H-furo[2,3-g]indazol-1-yl)propan-2-amine); (14) milnacipran; (15) citalopram; (16) desmethylsertraline (a metabolite of sertraline); (17) norfluoxetine; (18) desmethylcitalopram (a metabolite of citalopram); (19) escitalopram; (20) femoxetine; (21) ifoxetine; (22) cyanodothiepin; (23) litoxetine; (24) dapoxetine; (25) nefazodone; (26) cericlamine; (27) trazodone; (28) mirtazapine; (29) indalpine; (30) indeloxazine; (31) sibutramine; (32) zimeldine; (33) (+)-N-[N-[1-[1-(4-chlorophenyl)cyclobutyl]-3-methylbutyl}-N-methylamine; (34) (−)-N-{1-[1-(4-chlorophenyl)cyclobutyl-3-methylbutyl}-N-methylamine; (35) (−)-1-[1-(4-chlorophenyl)cyclobutyl]-3-methylbutylamine; (36) (+)-N-{1-[1-(4-chlorophenyl)cyclobutyl]-3-methylbutyl}-N; (37) (−)-N-{1-[1-(4-chlorophenyl)cyclobutyl]-3-methylbutyl}-N,N-dimethylamine)N-dimethylamine; (38) venlafaxine; (39) 0-desmethylvenlafaxine (a metabolite of venlafaxine); (40) clomipramine; (41) desmethylclomipramine (a metabolite of clomipramine); (42) buspirone; (43) olanzapine; (44) ziprasidone; (45) ergoloid mesylates; (46) pergolide mesylate; (47) vitamin B1; (48) vitamin B3; (49) vitamin B6; (50) biotin; (51) S-adenosylmethionine; (52) folic acid; (53) folinic acid; (54) ascorbic acid; (55) magnesium; (56) coenzyme Q10; (57) piracetam; (58) (+)-2,5-dimethoxy-4-iodoamphetamine; (59) (+)-3,4-methylenedioxyamphetamine; (60) (+)-N-[2-[4-[2,3-dihydro-2-(hydroxymethyl)-1,4-benzodioxin-5-yl]1-piperazinyl]-4-fluorobenzamide hydrochloride; (61) (+)-norfenfluramine (a metabolite of fenfluramine); (62) (3β)-2,3-dihydrolysergene; (63) (3β)-2,3-dihydrolysergol; (64) (3β)-2,3-dihydro-methyllysergate; (65) (3β, 5β, 8β)-9,10-didehydro-2,3-dihydro-6-methyl-8-(2-pyridylthiomethyl) ergoline; (66) (3β, 5β, 8β)-9,10-didehydro-2,3-dihydro-6-methyl-8-(methylthiomethyl) ergoline; (67) (3β, 5β, 8β)-9,10-didehydro-2,3-dihydro-6-methyl-8-(phenylthiomethyl) ergoline; (68) (3β, 5β, 8β)-9,10-didehydro-2,3-dihydro-8-methyl-6-propylergoline; (69) 1-(4-bromo-2,5-dimethoxyphenyl)-2-aminopropane; (70) 1-(m-trifluoromethylphenyl)-piperazine; (71) 2-(4-(4-(2-pyrimidinyl)1-piperazinyl-propyl)-1,2-benzoisothiazol-3-(2H)-one 1,1-dioxide hydrochloride; (72) 2-methylserotonin; (73) 3β, 5β, 8β)-9,10-didehydro-2,3-dihydro-6-methylergoline-8-acetonitrile; (74) zolmitriptan; (75) 3a,4,4a,6a,7,7a-hexahydro-2-[4-[4-(2-pyrimidinyl)-11-piperazinyl]butyl]-4,7-etheno-1H-cyclobutanoisoindole-1,3(2H)-dione dihydrochloride sesquihydrate; (76) 3-butyl-9,9-dimethyl-7-[4-[4-[2-methoxyphenyl) 1-piperazinyl]butyl]-3,7-diazabicyclo[3,2,1]nonane-2,4,6,8-tetraone; (77) 4,4-dimethyl-1-[4-[4-(2-pyrimidinyl)-1-piperazinyl]butyl]2,6-piperidinedione hydrochloride; (78) 5-hydroxy-L-tryptophan; (79) 5-methoxy-N,N-dimethyltryptamine; (80) 6-[[3-[4[o-methoxyphenyl]-1-piperazinyl]propyl]-amino]-1,3-dimethyluracil; (81) 8-[4-N-[4-(2-pyrimidinyl)-1-piperazinyl]-butyl]-8-azaspiro[4.5]-decane-7,9-dione hydrochloride; (82) 8-hydroxy-2-(di-n-propylamino)tetralin (8-OH-DPAT); (83) alniditan; (84) almotriptan; (85) 2-aminotetralin; (86) bifeprunox; (87) gepirone; (88) BW723C86 (1-[5(2-thienylmethoxy)-1H-3-indolyl[propan-2-amine hydrochloride); (89) cisapride; (90) dihydroergotamine; (91) D-lysergic acid diethylamide; (92) donitriptan; (93) eletriptan; (94) frovatriptan; (95) tegaserod; (96) ipsapirone; (97) L694247 (2-[5-[3-(4-methylsulphonylamino)benzyl-1,2,4-oxadiazol-5-yl]-1H-indol-3yl]ethanamine); (98) cinitapride; (99) lesopitron; (100) MCPP (m-chlorophenylpiperazine); (101) methysergide; (102) metoclopramide; (103) MK-212 (6-chloro-2-(1-piperazinyl)pyrazine hydrochloride); (104) mosapride; (105) N,N-dimethyl-5-methoxytryptamine; (106) N,N-dimethyltryptamine; (107) N-[4-[4-(2-pyrimidinyl)-1-piperazinyl]butylbicyclo[2.2.1]heptane-2,3-di-oxo-carboximide; (108) naratriptan; (109) norcisapride; (110) phentermine; (111) quipazine; (112) prucalopride; (113) rauwolscine; (114) repinotan; (115) rizatriptan; (116) sumatriptan; (117) tandospirone; (118) 1-methyl-4-phenyl-1,2,3,6-tetrahydropyridine; (119) tiaspirone; (120) trifluoromethylphenylpiperazine; (121) L-tryptophan; (122) xaliproden; (123) yohimbine; (124) zacopride; (125) zalospirone (126) mianserin; (127) setiptiline; (128) adatanserin; (129) altanserin; (130) benanserin; (131) blonanserin; (132) butanserin; (133) cinanserin; (134) eplivanserin; (135) flibanserin (136) glemanserin; (137) iferanserin; (138) ketanserin; (139) lidanserin; (140) pelanserin; (141) pruvanserin; (142) ritanserin; (143) seganserin; (144) tropanserin; (145) iloperidone; (146) sertindole; (147) EMR-62218; (148) asenapine; (149) zotepine; (150) ocaperidone; (151) APD125; (152) AVE8488; (153) pimavanserin; (154) isocarboxazid; (155) phenelzine; (156) tranylcypromine; (157) amitriptyline; (158) clomipramine; (159) N-(1-(1-methylethyl)piperidin-4-yl)-N-((4-methylphenyl)methyl)-4-methoxyphenylacetamide; (160) N-(1-(2,2-dimethylethyl)piperidin-4-yl)-N-((4-methylphenyl)methyl)-4-methoxyphenylacetamide; (161) N-(1-pentylpiperidin-4-yl)-N-((4-methylphenyl)methyl)-4-methoxyphenylacetamide; (162) N-(1-hexylpiperidin-4-yl)-N-((4-methylphenyl)methyl)-4-methoxyphenylacetamide; (163) N-(1-cyclohexylpiperidin-4-yl)-N-((4-methylphenyl)methyl)-4-methoxyphenylacetamide; (164) N-(1-cyclopentylpiperidin-4-yl)-N-((4-methylphenyl)methyl)-4-methoxyphenylacetamide; (165) N-(1-cyclobutylpiperidin-4-yl)-N-((4-methylphenyl)methyl)-4-methoxyphenylacetamide; (166) N-(1-cyclopropylpiperidin-4-yl)-N-((4-methylphenyl)methyl)-4-methoxyphenylacetamide; (167) N-(1-(cyclopentylmethyl)piperidin-4-yl)-N-((4-methylphenyl)methyl)-4-methoxyphenylacetamide; (168) N-(1-(cyclobutylmethyl)piperidin-4-yl)-N-((4-methylphenyl)methyl)-4-methoxyphenylacetamide; (169) N-(1-(cyclopropylmethyl)piperidin-4-yl)-N-((4-methylphenyl)methyl)-4-methoxyphenylacetamide; (170) N-(1-(2-hydroxyethyl)piperidin-4-yl)-N-((4-methylphenyl)methyl)-4-methoxyphenylacetamide; (171) N-(1-(3-hydroxypropyl)piperidin-4-yl)-N-((4-methylphenyl)methyl)-4-methoxyphenylacetamide; (172) N-((4-Methylphenyl)methyl)-N-(piperidin-4-yl)-N′-phenylmethylcarbamide; (173) N-((4-Methylphenyl)methyl)-N-(1-(2-methylpropyl)piperidin-4-yl)-N′-phenylmethylcarbamide; (174) N-(1-((2-Bromophenyl)methyl)piperidin-4-yl)-N-((4-methylphenyl)methyl)-N′-phenylmethylcarbamide; (175) N-(1-((4-Hydroxy-3-methoxyphenyl)methyl)piperidin-4-yl)-N-((4-methylphenyl)methyl)-N′-phenylmethylcarbamide; (176) N-(1-((5-Ethylthien-2-yl)methyl)piperidin-4-yl)-N-((4-methylphenyl)methyl)-N′-phenylmethylcarbamide; (177) N-(1-(Imidazol-2-ylmethyl)piperidin-4-yl)-N-((4-methylphenyl)methyl)-N′-phenylmethylcarbamide; (178) N-(1-(Cyclohexylmethyl)piperidin-4-yl)-N-((4-methylphenyl)methyl)-N′-phenylmethylcarbamide; (179) N-(1-((4-Fluorophenyl)methyl)piperidin-4-yl)-N-((4-methylphenyl)methyl)-N′-phenylmethylcarbamide; (180) N-((4-Methylphenyl)methyl)-N-(piperidin-4-yl)-4-methoxyphenylacetamide; (181) N-((4-Methylphenyl)methyl)-N-(1-methylpiperidin-4-yl)-4-methoxyphenylacetamide; (182) N-(1-Ethylpiperidin-4-yl)-N-((4-methylphenyl)methyl)-4-methoxyphenylacetamide; (183) N-((4-Methylphenyl)methyl)-N-(1-propylpiperidin-4-yl)-4-methoxyphenylacetamide; (184) N-(1-Butylpiperidin-4-yl)-N-((4-methylphenyl)methyl)-4-methoxyphenylacetamide; (185) N-(1-(3,3-Dimethylbutyl)piperidin-4-yl)-N-((4-methylphenyl)methyl)-4-methoxyphenylacetamide; (186) N-(1-(Cyclohexylmethyl)piperidin-4-yl)-N-((4-methylphenyl)methyl)-4-methoxyphenylacetamide; (187) N-((4-Methylphenyl)methyl)-N-(1-(2-methylpropyl)piperidin-4-yl)-4-methoxyphenylacetamide; (188) N-((4-Methylphenyl)methyl)-N-(1-((4-methylphenyl)methyl)piperidin-4-yl)-4-methoxyphenylacetamide; (189) N-(1-((4-Hydroxyphenyl)methyl)piperidin-4-yl)-N-((4-methylphenyl)methyl)-4-methoxyphenylacetamide; (190) N-(1-((2-Hydroxyphenyl)methyl)piperidin-4-yl)-N-((4-methylphenyl)methyl)-4-methoxyphenylacetamide; (191) N-(3-Phenylpropyl)-N-(piperidin-4-yl)-4-methoxyphenylacetamide; (192) N-(2-Phenylethyl)-N-(piperidin-4-yl)-4-methoxyphenylacetamide; (193) N-((2-Methoxyphenyl)methyl)-N-(piperidin-4-yl)-4-methoxyphenylacetamide; (194) N-((2-Chlorophenyl)methyl)-N-(piperidin-4-yl)-4-methoxyphenylacetamide; (195) N-((3,4-Di-methoxyphenyl)methyl)-N-(piperidin-4-yl)-4-methoxyphenylacetamide; (196) N-((4-Fluorophenyl)methyl)-N-(piperidin-4-yl)-4-methoxyphenylacetamide; (197) N-((2,4-Di-chlorophenyl)methyl)-N-(piperidin-4-yl)-4-methoxyphenylacetamide; (198) N-((3-Methylphenyl)methyl)-N-(piperidin-4-yl)-4-methoxyphenylacetamide; (199) N-((3-Bromophenyl)methyl)-N-(piperidin-4-yl)-4-methoxyphenylacetamide; (200) N-(1-(Phenylmethyl)piperidin-4-yl)-N-(3-phenyl-2-propen-1-yl)-4-methoxyphenylacetamide; (201) N-((4-Methylphenyl)methyl)-N-(1-piperidin-4-yl)-phenylacetamide; (202) N-((4-Methylphenyl)methyl)-N-(1-piperidin-4-yl)-3-phenylpropionamide; (203) N-((4-Methylphenyl)methyl)-N-(1-piperidin-4-yl)-(phenylthio)acetamide; (204) N-((4-Methylphenyl)methyl)-N-(1-piperidin-4-yl)-phenoxyacetamide; (205) N-((4-Methylphenyl)methyl)-N-(1-piperidin-4-yl)-(4-chlorophenoxy)acetamide; (206) N-((4-Methylphenyl)methyl)-N-(1-piperidin-4-yl)-3-methoxyphenylacetamide; (207) N-((4-Methylphenyl)methyl)-N-(1-piperidin-4-yl)-4-fluorophenylacetamide; (208) N-((4-Methylphenyl)methyl)-N-(1-piperidin-4-yl)-2,5-di-methoxyphenylacetamide; (209) N-((4-Methylphenyl)methyl)-N-(1-piperidin-4-yl)-4-chlorophenylacetamide; (210) N-((4-Methylphenyl)methyl)-N-(1-(phenylmethyl)pyrrolidin-3-yl)-N′-phenylmethylcarbamide; (211) N-((4-Methylphenyl)methyl)-N-(1-(phenylmethyl)pyrrolidin-3-yl)-4-methoxyphenylacetamide; (212) 2-(4-methoxyphenyl)-N-(4-methylbenzyl)-N-(piperidin-4-yl)acetamide; (213) 2-(4-methoxyphenyl)-N-(4-methylbenzyl)-N-(1-methylpiperidin-4-yl)acetamide; (214) 2-(4-methoxyphenyl)-N-(4-methylbenzyl)-N-(1-ethylpiperidin-4-yl)acetamide; (215) 2-(4-methoxyphenyl)-N-(4-chlorbenzyl)-N-(1-ethylpiperidin-4-yl)acetamide; (216) 2-(4-methoxyphenyl)-N-(4-chlorbenzyl)-N-(1-isopropylpiperidin-4-yl)acetamide; (217) 2-(4-methoxyphenyl)-N-(4-chlorobenzyl)-N-(piperidin-4-yl)acetamide; (218) 2-(4-methoxyphenyl)-N-(4-chlorobenzyl)-N-(1-cyclopentylpiperidin-4-yl)acetamide; (219) 2-(4-methoxyphenyl)-N-(4-chlorbenzyl)-N-(1-isopropylpiperidin-4-yl)acetamide; (220) 2-(phenyl)-N-(4-trifluoromethylbenzyl)-N-(1-methylpiperidin-4-yl)acetamide; (221) 2-(4-fluorophenyl)-N-(4-trifluoromethylbenzyl)-N-(1-methylpiperidin-4-yl)acetamide; (222) 2-(4-Methoxyphenyl)-N-(4-trifluoromethylbenzyl)-N-(1-methylpiperidin-4-yl)acetamide; (223) 2-(4-Trifluoromethylphenyl)-N-(4-trifluoromethylbenzyl)-N-(1-methylpiperidin-4-yl)acetamide; (224) 2-(4-Fluorophenyl)-N-(4-fluorobenzyl)-N-(1-methylpiperidin-4-yl)acetamide; (225) 2-(4-Methoxyphenyl)-N-(4-fluorobenzyl)-N-(1-methylpiperidin-4-yl)acetamide; (226) 2-(phenyl)-N-(4-fluorobenzyl)-N-(1-methylpiperidin-4-yl)acetamide; (227) 2-(4-Trifluoromethylphenyl)-N-(4-fluorobenzyl)-N-(1-methylpiperidin-4-yl)acetamide; (228) 2-(4-trifluoromethylphenyl)-N-[4-(methoxycarbonyl)benzyl]-N-(1-methylpiperidin-4-yl)acetamide; (229) 2-Phenyl-N-[4-(methoxycarbonyl)benzyl]-N-(1-methylpiperidin-4-yl)acetamide; (230) 2-(4-Chlorophenyl)-N-[4-(methoxycarbonyl)benzyl]-N-(1-methylpiperidin-4-yl) acetamide; (231) 2-(4-Methoxyphenyl)-N-[4-(methoxycarbonyl)benzyl]-N-(1-methylpiperidin-4-yl)acetamide; (232) 2-(4-trifluoromethylphenyl)-N-[4-(methoxycarbonyl)benzyl]-N-(1-methylpiperidin-4-yl)acetamide; (233) 2-Phenyl-N-[4-(methoxycarbonyl)benzyl]-N-(1-methylpiperidin-4-yl)acetamide; (234) 2-(4-Chlorophenyl)-N-[4-(methoxycarbonyl)benzyl]-N-(1-methylpiperidin-4-yl) acetamide; (235) 2-(4-Methoxyphenyl)-N-[4-(methoxycarbonyl)benzyl]-N-(1-methylpiperidin-4-yl)acetamide; (236) 2-(4-methoxyphenyl)-N-(4-methylbenzyl)-N-[1-(4-chloromethyl-2-thiazolylmethyl)piperidin-4-yl]acetamide; (237) 2-(4 methoxyphenyl)-N-(4-methylbenzyl)-N-{1-[3-(1,3-dihydro-2H-benzimidazol-2-on-1-yl)propyl]piperidin-4-yl}acetamide; (238) 2-(4-methoxyphenyl)-N-(2-4(fluorophenyl)ethyl)-N-(1-methylpiperidin-4-yl)acetamide; (239) 2-(4-methoxyphenyl)-N-[2-(2,5-dimethoxyphenyl)ethyl]-N-(1-methylpiperidin-4-yl)acetamide; (240) 2-(4-methoxyphenyl)-N-[2-(2,4-dichlorophenyl)ethyl]-N-(1-methylpiperidin-4-yl)acetamide; (241) 2-(4-methoxyphenyl)-N-[2-(3-chlorophenyl)ethyl]-N-(1-methylpiperidin-4-yl)acetamide; (242) 2-(4-methoxyphenyl)-N-[2-(4-methoxyphenyl)ethyl]-N-(1-methylpiperidin-4-yl) acetamide; (243) 2-(4-methoxyphenyl)-N-[2-(3-fluorophenyl)ethyl]-N-(1-methylpiperidin-4-yl)acetamide; (244) 2-(4-ethoxyphenyl)-N-[2-(4-fluorophenethyl]-N-(1-methylpiperidin-4-yl)acetamide; (245) 2-(4-ethoxyphenyl)-N-(4-fluorobenzyl)-N-(1-methylpiperidin-4-yl)acetamide; (246) 2-(4-methoxyphenyl)-N-(4-methylbenzyl)-N-{1-[2-(2-hydroxyethoxy)ethyl]piperidin-4-yl}acetamide; (247) 2-(4-methoxyphenyl)-N-(4-methylbenzyl)-N-[1-((2-chloro-5-thienyl)methyl) piperidin-4-yl]acetamide; (248) 2-(4-methoxyphenyl)-N-(4-methylbenzyl)-N-[1-(2-(imidazolidinon-1-yl)ethyl)piperidin-4-yl]acetamide; (249) 2-(4-methoxyphenyl)-N-(4-methylbenzyl)-N-{1-[2-(2,4(1H,3H)quinazolinedion-3-yl)ethyl]piperidin-4-yl}acetamide; (250) 2-(4-methoxyphenyl)-N-(4-methylbenzyl)-N-{1-[2-(1,3-dioxolan-2-yl)ethyl]piperidin-4-yl}acetamide; (251) 2-(4-methoxyphenyl)-N-(4-methylbenzyl)-N-{1-[2-(3-indolyl)ethyl]piperidin-4-yl}acetamide; (252) 2-(4-methoxyphenyl)-N-(4-methylbenzyl)-N-{1-[3-(1,2,4-triazol-1-yl)propyl]piperidin-4-yl}acetamide; (253) 2-(4-methoxyphenyl)-N-(4-methylbenzyl)-N-[1-(5-benzofurazanylmethyl)piperidin-4-yl]acetamide; (254) 2-(4-methoxyphenyl)-N-(4-methylbenzyl)-N-[1-(5-chlorobenzo[b]thien-3-ylmethyl)piperidin-4-yl]acetamide; (255) 2-(4-methoxyphenyl)-N-(4-methylbenzyl)-N-[1-(5-phenyl-1,2,4-oxadiazol-3-ylmethyl)piperidin-4-yl]acetamide; (256) 2-(4-Chlorophenyl)-N-(4-methylbenzyl)-N-(1-isopropylpiperidin-4-yl)-acetamide; (257) 2-(4-Chlorophenyl)-N-(4-methylbenzyl)-N-(1-ethylpiperidin-4-yl)-acetamide; (258) 2-Phenyl-N-(4-methylbenzyl)-N-(1-methylpiperidin-4-yl)-acetamide; (259) 2-(4-Chlorophenyl)-N-(4-methylbenzyl)-N-(1-methylpiperidin-4-yl)-acetamide; (260) 2-(4-Chlorophenyl)-N-(4-methylbenzyl)-N-(1-cyclopentylpiperidin-4-yl)-acetamide; (261) 2-(4-Fluorophenyl)-N-(4-methylbenzyl)-N-(1-methylpiperidin-4-yl)-acetamide; (262) 2-(4-Chlorophenyl)-N-(4-methylbenzyl)-N-(1-(2-hydroxyethyl)-piperidin-4-yl)-acetamide; (263) 2-(4-Chlorophenyl)-N-(4-methylbenzyl)-N-(1-cyclobutylpiperidin-4-yl)-acetamide; (264) 2-(4-Methoxyphenyl)-N-(4-methylbenzyl)-N-(1-cyclobutylpiperidin-4-yl)-acetamide; (265) 2-(4-Methoxyphenyl)-N-(4-methylbenzyl)-N-(tropin-4-yl)-acetamide; (266) N-(4-Methylbenzyl)-N-(1-methylpiperidin-4-yl)-N′-benzyl-carbamide; (267) N-(4-Methylbenzyl)-N-(1-methylpiperidin-4-yl)-N′-phenyl-carbamide; (268) N-Phenethyl-N-(1-methylpiperidin-4-yl)-N′-benzyl-carbamide; (269) 2-Phenyl-N-(4-methoxybenzyl)-N-(1-methylpiperidin-4-yl)-acetamide; (270) 2-(4-Trifluoromethylphenyl)-N-(4-methoxybenzyl)-N-(1-methylpiperidin-4-yl)-acetamide (271) 2-(4-Fluorophenyl)-N-(4-methoxybenzyl)-N-(1-methylpiperidin-4-yl)-acetamide; (272) 2-(4-Methoxyphenyl)-N-(4-methoxybenzyl)-N-(1-methylpiperidin-4-yl)-acetamide; (273) 2-(4-Methylphenyl)-N-(4-chlorobenzyl)-N-(1-methylpiperidin-4-yl)-acetamide; (274) 2-(4-Hydroxyphenyl)-N-(4-methylbenzyl)-N-(1-methylpiperidin-4-yl)-acetamide; (275) N-Phenethyl-N-(1-methylpiperidin-4-yl)-N′-phenyl-carbamide; (276) N-(3-Phenylpropyl)-N-(1-methylpiperidin-4-yl)-N′-benzyl-carbamide; (277) N-(3-Phenylpropyl)-N-(1-methylpiperidin-4-yl)-N′-phenyl-carbamide; (278) 2-(4-Methoxyphenyl)-2,2-ethylene-N-(4-methylbenzyl)-N-(1-methylpiperidin-4-yl)acetamide; (279) 2-(4-Methoxyphenyl)-N-alpha-methylbenzyl-N-(1-methylpiperidin-4-yl)acetamide; (280) 2-(4-Methoxyphenyl)-N-(4-methylbenzyl)-N-(3-tropen-4-yl)acetamide; (281) 2-Phenyl-2-ethyl-N-(4-methylbenzyl)-N-(1-methylpiperidin-4-yl)acetamide; (282) N-Phenethyl-N-(4-methylbenzyl)-N-(1-methylpiperidin-4-yl)-amine; (283) 2-(4-Methoxyphenyl)-N-(1-indanyl)-N-(1-methylpiperidin-4-yl)acetamide; (284) N-(4-Methylbenzyl)-N-(1-methylpiperidin-4-yl)-N′-(4-methoxybenzyl)-carbamide; (285) 2-(3,4-dimethoxyphenyl)-N-(4-methylbenzyl)-N-(1-methylpiperidin-4-yl)acetamide; (286) 2-(3,4-Methylenedioxyphenyl)-N-(4-methylbenzyl)-N-(1-methylpiperidin-4-yl)acetamide; (287) 2-(4-Methoxyphenyl)-N-(4-methylbenzyl)-N-(1-t-butylpiperidin-4-yl)-acetamide; (288) N-(4-Methylbenzyl)-N-(1-methylpiperidin-4-yl)-N′-phenethyl-carbamide; (289) N-Phenethyl-N-(1-methylpiperidin-4-yl)-N′-phenethyl-carbamide; (290) N-(4-Methylbenzyl)-N-(1-t-butylpiperidin-4-yl)-N′-(4-methoxybenzyl)-carbamide; (291) 2-(4-Ethoxyphenyl)-N-(4-methylbenzyl)-N-(1-methylpiperidin-4-yl)acetamide; (292) 2-(4-Butoxyphenyl)-N-(4-methylbenzyl)-N-(1-methylpiperidin-4-yl)acetamide; (293) 2-(4-i-Propoxyphenyl)-N-(4-methylbenzyl)-N-(1-methylpiperidin-4-yl)acetamide; (294) 2-(4-t-Butoxyphenyl)-N-(4-methylbenzyl)-N-(1-methylpiperidin-4-yl)acetamide; (295) 2-(4-Butoxyphenyl)-N-(4-fluorobenzyl)-N-(1-methylpiperidin-4-yl)acetamide; (296) 2-(4-Propoxyphenyl)-N-(4-fluorobenzyl)-N-(1-methylpiperidin-4-yl)acetamide; (297) 2-(4-i-Propoxyphenyl)-N-(4-fluorobenzyl)-N-(1-methylpiperidin-4-yl)acetamide; (298) 2-(4-t-Butoxyphenyl)-N-(4-fluorobenzyl)-N-(1-methylpiperidin-4-yl)acetamide; (299) 4-(4-Fluorobenzyl)-3-(4-methoxybenzyl)-8-methyl-1-oxa-3,8-diaza-spiro[4.5]decan-2-one; (300) 3-(4-Ethoxybenzyl)-4-(4-fluorobenzyl)-8-methyl-1-oxa-3,8-diaza-spiro[4.5]decan-2-one; (301) 4-(4-Fluorobenzyl)-8-methyl-3-(4-propoxybenzyl)-1-oxa-3,8-diaza-spiro[4.5]decan-2-one; (302) 3-(4-Cyclopropylmethoxybenzyl)-4-(4-fluorobenzyl)-8-methyl-1-oxa-3,8-diaza-spiro[4.5]decan-2-one; (303) 4-(4-Fluorobenzyl)-3-(4-isopropoxybenzyl)-8-methyl-1-oxa-3,8-diaza-spiro[4.5]decan-2-one; (304) 3-(4-Butoxybenzyl)-4-(4-fluorobenzyl)-8-methyl-1-oxa-3,8-diaza-spiro[4.5]decan-2-one; (305) 4-(4-Fluorobenzyl)-3-(4-isobutoxybenzyl)-8-methyl-1-oxa-3,8-diaza-spiro[4.5]decan-2-one; (306) 3-(4-Difluoromethoxybenzyl)-4-(4-fluorobenzyl)-8-methyl-1-oxa-3,8-diaza-spiro[4.5]decan-2-one; (307) 4-(4-Fluorobenzyl)-8-methyl-3-(4-trifluoromethoxybenzyl)-1-oxa-3,8-diaza-spiro[4.5]decan-2-one; (308) 4-(4-Fluorobenzyl)-8-methyl-3-(4-pentoxybenzyl)-1-oxa-3,8-diaza-spiro[4.5]decan-2-one; (309) 8-Ethyl-4-(4-fluorobenzyl)-3-(4-isobutoxybenzyl)-1-oxa-3,8-diaza-spiro[4.5]decan-2-one; (310) 4-(4-Fluorobenzyl)-3-(4-isobutoxybenzyl)-8-isopropyl-1-oxa-3,8-diaza-spiro[4.5]decan-2-one; (311) 8-Cyclopropylmethyl-4-(4-fluorobenzyl)-3-(4-isobutoxybenzyl)-1-oxa-3,8-diaza-spiro[4.5]decan-2-one; (312) 8-Cyclohexylmethyl-4-(4-fluorobenzyl)-3-(4-isobutoxybenzyl)-1-oxa-3,8-diaza-spiro[4.5]decan-2-one; (313) 8-Cyclopentyl-4-(4-fluorobenzyl)-3-(4-isobutoxybenzyl)-1-oxa-3,8-diaza-spiro[4.5]decan-2-one; (314) 4-(4-Fluorobenzyl)-3-(4-isobutoxybenzyl)-8-(3-morpholin-4-yl-propyl)-1-oxa-3,8-diaza-spiro[4.5]decan-2-one; (315) 8-(2-[1,3]Dioxolan-2-yl-ethyl)-4-(4-fluorobenzyl)-3-(4-isobutoxybenzyl)-1-oxa-3,8-diaza-spiro[4.5]decan-2-one; (316) 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one; (317) 4-(4-Fluorobenzyl)-3-(4-isobutoxybenzyl)-8-[3-(2-oxo-2,3-dihydro-benzoimidazol-1-yl)-propyl]-1-oxa-3,8-diaza-spiro[4.5]decan-2-one; (318) 4-(4-Fluorobenzyl)-3-(4-isobutoxybenzyl)-8-(2-methyl-thiazol-4-yl-methyl)-1-oxa-3,8-diaza-spiro[4.5]decan-2-one; (319) 4-(4-Chlorobenzyl)-3-(4-isobutoxybenzyl)-8-methyl-1-oxa-3,8-diaza-spiro[4.5]decan-2-one; (320) 8-Ethyl-4-(4-chlorobenzyl)-3-(4-isobutoxybenzyl)-1-oxa-3,8-diaza-spiro[4.5]decan-2-one; (321) 4-(4-Chlorobenzyl)-3-(4-isobutoxybenzyl)-8-isopropyl-1-oxa-3,8-diaza-spiro[4.5]decan-2-one; (322) 8-Cyclopropylmethyl-4-(4-chlorobenzyl)-3-(4-isobutoxybenzyl)-1-oxa-3,8-diaza-spiro[4.5]decan-2-one; (323) 8-Cyclohexylmethyl-4-(4-chlorobenzyl)-3-(4-isobutoxybenzyl)-1-oxa-3,8-diaza-spiro[4.5]decan-2-one; (324) 8-(2-[1,3]Dioxolan-2-yl-ethyl)-4-(4-chlorobenzyl)-3-(4-isobutoxybenzyl)-1-oxa-3,8-diaza-spiro[4.5]decan-2-one; (325) 4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one; (326) 3-(4-Difluoromethoxybenzyl)-4-(4-fluorobenzyl)-8-methyl-1-oxa-3,8-diaza-spiro[4.5]decan-2-one; (327) 3-(4-Difluoromethoxybenzyl)-8-ethyl-4-(4-fluorobenzyl)-1-oxa-3,8-diaza-spiro[4.5]decan-2-one; (328) 3-(4-Difluoromethoxybenzyl)-4-(4-fluorobenzyl)-8-isopropyl-1-oxa-3,8-diaza-spiro[4.5]decan-2-one; (329) 8-Cyclopropylmethyl-3-(4-difluoromethoxybenzyl)-4-(4-fluorobenzyl)-1-oxa-3,8-diaza-spiro[4.5]decan-2-one; (330) 8-Cyclohexylmethyl-3-(4-difluoromethoxybenzyl)-4-(4-fluorobenzyl)-1-oxa-3,8-diaza-spiro[4.5]decan-2-one; (331) 3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one; (332) 3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one; (333) 8-Ethyl-4-(4-fluorobenzyl)-3-(4-trifluoromethoxybenzyl)-1-oxa-3,8-diaza-spiro[4.5]decan-2-one; (334) 4-(4-Fluorobenzyl)-8-isopropyl-3-(4-trifluoromethoxybenzyl)-1-oxa-3,8-diaza-spiro[4.5]decan-2-one; (335) 8-Cyclopropylmethyl-4-(4-fluorobenzyl)-3-(4-trifluoromethoxybenzyl)-1-oxa-3,8-diaza-spiro[4.5]decan-2-one; (336) 8-Cyclohexylmethyl-4-(4-fluorobenzyl)-3-(4-trifluoromethoxybenzyl)-1-oxa-3,8-diaza-spiro[4.5]decan-2-one; (337) 8-Cyclopentyl-4-(4-fluorobenzyl)-3-(4-trifluoromethoxybenzyl)-1-oxa-3,8-diaza-spiro[4.5]decan-2-one; (338) 8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one; (339) 4-(4-Fluorobenzyl)-8-[2-(2-oxo-imidazolidin-1-yl)-ethyl]-3-(4-trifluoromethoxybenzyl)-1-oxa-3,8-diaza-spiro[4.5]decan-2-one; (340) 8-Ethyl-4-(4-fluorobenzyl)-3-(4-propoxybenzyl)-1-oxa-3,8-diaza-spiro[4.5]decan-2-one; (341) 4-(4-Fluorobenzyl)-8-isopropyl-3-(4-propoxybenzyl)-1-oxa-3,8-diaza-spiro[4.5]decan-2-one; (342) 8-Cyclopropylmethyl-4-(4-fluorobenzyl)-3-(4-propoxybenzyl)-1-oxa-3,8-diaza-spiro[4.5]decan-2-one; (343) 8-Cyclohexylmethyl-4-(4-fluorobenzyl)-3-(4-propoxybenzyl)-1-oxa-3,8-diaza-spiro[4.5]decan-2-one; (344) 8-Cyclopentyl-4-(4-fluorobenzyl)-3-(4-propoxybenzyl)-1-oxa-3,8-diaza-spiro[4.5]decan-2-one; (345) 8-(2-[1,3]Dioxolan-2-yl-ethyl)-4-(4-fluorobenzyl)-3-(4-propoxybenzyl)-1-oxa-3,8-diaza-spiro[4.5]decan-2-one; (346) 4-(4-Fluorobenzyl)-8-[2-(2-oxo-imidazolidin-1-yl)-ethyl]-3-(4-propoxybenzyl)-1-oxa-3,8-diaza-spiro[4.5]decan-2-one; (347) 3-(4-Cyclopropylmethoxybenzyl)-8-ethyl-4-(4-fluorobenzyl)-1-oxa-3,8-diaza-spiro[4.5]decan-2-one; (348) 3-(4-Cyclopropylmethoxybenzyl)-4-(4-fluorobenzyl)-8-isopropyl-1-oxa-3,8-diaza-spiro[4.5]decan-2-one; (349) 3-(4-Cyclopropylmethoxybenzyl)-8-cyclopropylmethyl-4-(4-fluorobenzyl)-1-oxa-3,8-diaza-spiro[4.5]decan-2-one; (350) 3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one; (351) 3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one; (352) 8-(2-[1.3]-Dioxan-2-yl-ethyl)-4-(4-fluorobenzyl)-3-(4-isobutoxybenzyl)-1-oxa-3,8-diaza-spiro[4.5]decane-3-one; (353) 4-(4-Fluorobenzyl)-3-(4-isobutoxybenzyl)-8-{3-[(S)-4-isopropyl-2-oxo-oxazolidin-3-yl]-propyl}-1-oxa-3,8-diaza-spiro[4.5]decane-3-one; (354) N-{1-[2-(1,3-Dioxolan-2-yl)ethyl]piperidin-4-yl}-N-(4-fluorobenzyl)-W-(4-isobutoxybenzyl)carbamide hydrochloride; (355) N-{1-[2-(1,3-Dioxan-2-yl)ethyl]-piperidin-4-yl}-N-(4-fluorobenzyl)-2-[4-(2-hydroxy-2-methylpropoxy)phenyl]-acetamide tartrate; (356) N-(4-Fluorobenzyl)-N-(piperidin-4-yl)-2-(4-isobutoxyphenyl)acetamide; (357) N-{1-[3-(3,5-Dimethylpiperidin-1-yl)propyl]piperidin-4-yl-}-N-(4-fluorobenzyl)-2-(4-isobutoxyphenyl)acetamide dihydrochloride; (358) 1-[3-(4-{(4-Fluorobenzyl)-[2-(4isobutoxyphenyl)acetyl]amino}piperidin-1-yl)propyl]piperidine-4-carboxylic acid methyl ester dihydrochloride; (359) N-(4-Fluorobenzyl)-2-(4-isobutoxyphenyl)-N-{1-[2-(1-methylpyrrolidin-2-yl-)ethyl]piperidin-4-yl}acetamide dioxalate; (360) N-{1-[3-(2,6-Dimethylmorpholin-4-yl)propyl]piperidin-4-yl}-N-(4-fluorobenzyl)-2-(4-isobutoxyphenyl)acetamide dioxalate; (361) N-(4-Fluorobenzyl)-N-{1-[3-(3-hydroxypiperidin-1-yl)propyl]piperidin-4-yl}-2-(4-isobutoxyphenyl)acetamide dioxalate; (362) N-(4-Fluorobenzyl)-2-(4-isobutoxyphenyl)-N-{1-[3-(2-methylpiperidin-1-yl)-propyl]piperidin-4-yl}acetamide dioxalate; (363) N-(4-Fluorobenzyl-2-(4-isobutoxyphenyl)-N-[1-(3-pyrrolidin-1-yl-propyl)piperidin-4-yl]acetamide dioxalate; (364) N-{1-[3-(2,5-Dimethylpyrrolidin-1-yl)propyl]piperidin-4-yl}-N-(4-fluorobenzyl)-2-(4-isobutoxyphenyl)acetamide dioxalate; (365) N-(4-Fluorobenzyl)-N-{1-[3-(3-hydroxymethylpiperidin-1-yl)propyl]piperidin-4-yl}-2-(4-isobutoxyphenyl)acetamide dioxalate; (366) N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide oxalate; (367) N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide oxalate; (368) N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide oxalate; (369) N-(4-Fluorobenzyl)-N-{1-[3-(4-(S)-isopropyl-2-oxo-oxazolidin-3-yl)propyl]piperidin-4-yl}-2-(4-propoxyphenyl)acetamide oxalate; (370) N-{1-[2-(1,3-Dioxan-2-yl)ethyl]piperidin-4-yl}-N-(4-fluorobenzyl)-2-(4-isobutoxyphenyl)acetamide oxalate; (371) N-{1-[2-(1,3-Dioxan-2-yl)ethyl]piperidin-4-yl}-N-[2-(4-fluorophenyl)ethyl]-2-(4-isobutoxyphenyl)acetamide oxalate; (372) N-{1-[2-(1,3-Dioxan-2-yl)ethyl]piperidin-4-yl}-N-[2-(4-fluorophenyl)ethyl]-2-(4-propoxyphenyl)acetamide oxalate; (373) N-{1-[2-(1,3-Dioxan-2-yl-)ethyl]piperidin-4-yl}-N-(4-fluorobenzyl)-2-(4-propoxyphenyl)acetamide tartrate; (374) N-{1-[2-(1,3-Dioxan-2-yl)ethyl]piperidin-4-yl}-N-(4-fluorobenzyl)-N′-(4-isobutoxybenzyl)carbamide tartrate; (375) N-{1-[2-(1,3-Dioxan-2-yl)ethyl]piperidin-4-yl}-N-(4-fluorobenzyl)-2-(4-fluorophenyl)acetamide tartrate; (376) N-{1-[2-(1,3-Dioxan-2-yl)ethyl]piperidin-4-yl}-N-(4-fluorobenzyl)-2-p-tolylacetamide tartrate; (377) 2-Benzofuran-5-yl-N-{1-[2-(1,3-dioxan-2-yl)ethyl]piperidin-4-yl}-N-(4-fluorobenzyl)acetamide tartrate; (378) 2-(2,3-Dihydrobenzofuran-5-yl)-N-{1-[2-(1,3-dioxan-2-yl)ethyl]piperidin-4-yl}-N-(4-fluorobenzyl)acetamide tartrate; (379) N-{1-[2-(2,2-Dimethyl-1,3-dioxolan-4-yl)ethyl]piperidin-4-yl}-N-(4-fluorobenzyl)-2-(4-isobutoxyphenyl)acetamide tartrate; (380) N-{1-[2-(1,3-Dioxan-4-yl)ethyl]piperidin-4-yl}-N-(4-fluorobenzyl)amine; (381) N-{1-[2-(1,3-Dioxan-4-yl)ethyl]piperidin-4-yl}-N-(4-fluorobenzyl)-2-(4-isobutoxyphenyl)acetamide tartrate; (382) N-{1-[2-(1,3-Dioxan-4-yl)ethyl]piperidin-4-yl}-N(4-fluorobenzyl)-2-(4-trifluoromethylphenyl)acetamide tartrate; (383) 2-(4-Cyanophenyl)-N-{1-[2-(1,3-dioxan-4-yl)ethyl]piperidin-4-yl}-N-(4-fluorobenzyl)acetamide tartrate; (384) N-(4-Fluorobenzyl)-2-(4-isobutoxyphenyl)-N-{1-[2-(2-oxo-imidazolidin-1-yl)ethyl]piperidin-4-yl}acetamide hydrochloride; (385) 2-(4-Methoxyphenyl)-N-(4-methylbenzyl)-N-{1-[2-(2-oxo-imidazolidin-1-yl)ethyl]piperidin-4-yl}acetamide hydrochloride; (386) N-(4-Fluorobenzyl)-2-(4-isopropoxyphenyl)-N-{1-[2-(2-oxo-imidazolidin-1-yl)ethyl]piperidin-4-yl}acetamide hydrochloride; (387) N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide hydrochloride; (388) N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide hydrochloride; (389) 2-(4-Methoxyphenyl)-N-(4-methylbenzyl)-N-{1-[3-(2-oxo-2,3-dihydrobenzoimidazol-1-yl)propyl]piperidin-4-yl}-acetamide hydrochloride; (390) N-(4-Fluorobenzyl)-2-(4-isopropoxyphenyl)-N-{1-[4-(2-oxo-2,3-dihydrobenzoimidazol-1-yl)butyl]piperidin-4-yl}acetamide hydrochloride; (391) N-{1-[2-(2,4-Dioxo-1,4-dihydro-2H-quinazolin-3-ypethyl]piperidin-4-yl}-N-(4-fluorobenzyl)-2-(4-isopropoxyphenyl)acetamide hydrochloride; (392) 4-(4-Fluorobenzylamino)-piperidine-1-carboxylic acid benzyl ester; (393) N-(1-Benzyloxycarbonylpiperidin-4-yl)-N-(4-fluorobenzyl)-N′-(4-isopropoxybenzyl)carbamide; (394) N-(4-Fluorobenzyl)-N′-(4-isopropoxybenzyl)-N-piperidin-4-yl-carbamide oxalate; (395) N-{1-[2-(1,3-Dioxolan-2-yl)ethyl]piperidin-4-yl}-N-(4-fluorobenzyl)-N′-(4-isopropoxy-benzyl)carbamide oxalate; (396) N-{1-[2-(1,3-Dioxolan-2-yl)ethyl]piperidin-4-yl]2-(4-methoxyphenyl)-N-(4-methylbenzyl)acetamide hydrochloride; (397) N-{1-[2-(1,3-Dioxolan-2-yl-)ethyl]piperidin-4-yl}-N-(4-fluorobenzyl)-2-(4-isobutoxyphenyl)acetamide hydrochloride; (398) N-{1-[2-(1,3-Dioxolan-2-yl)ethyl]piperidin-4-yl}-2-(4-isopropoxyphenyl)-N-(4-methylbenzyl)acetamide hydrochloride; (399) N-{1-[2-(1,3-Dioxolan-2-yl)ethyl]piperidin-4-yl}-N-(4-fluorobenzyl)-2-(4-propoxyphenyl)acetamide tartrate; (400) N-(4-Fluorobenzyl)-N′-(4-isopropoxybenzyl)-N-{1-[2-((S)-4-methyl-1,3-dioxolane-2-yl)ethyl]piperidin-4-yl}carbamide oxalate; (401) N-(4-Fluorobenzyl)-N′-(4-isopropoxybenzyl)-N-[1-(3-morpholin-4-yl-propyl)piperidin-4-yl]carbamide oxalate; (402) 2-(4-Methoxyphenyl)-N-(4-methylbenzyl)-N-[1-(2-morpholin-4-yl-ethyl)piperidin-4-yl]acetamide dihydrochloride; (403) 2-(4-Methoxyphenyl)-N-(4-methylbenzyl)-N-[1-(3-morpholin-4-ylpropyl)piperidin-4-yl]acetamide dihydrochloride; (404) N-(4-Fluorobenzyl)-2-(4-isobutoxyphenyl)-N-[1-(3-morpholin-4-ylpropyl)piperidin-4-yl]acetamide dihydrochloride; (405) N-(4-Fluorobenzyl)-2-(4-isopropoxy-phenyl)-N-[1-(3-morpholin-4-yl-propyl)piperidin-4-yl]acetamide dihydrochloride; (406) N-(4-Fluorobenzyl)-N′-(4-isopropoxybenzyl)-N-[1-(3-piperidin-1-yl-propyl)piperidin-4-yl]carbamide oxalate; (407) N-(4-Fluorobenzyl)-N′-(4-isopropoxybenzyl)-N-[1-(3-((S)-4-isopropyl-2-oxazolidinon-1-yl-propyl)piperidin-4-yl]carbamide tartrate; (408) N-(4-Fluorobenzyl)-N′-(4-isopropoxybenzyl)-N-{1-[2-(2,5,5-trimethyl-1,3-dioxan-2-yl)ethyl]}piperidin-4-yl]carbamide oxalate; (409) N-{1-[3-(1,3-Dioxolan-2-yl)propyl]piperidin-4-yl}-N-(4-fluorobenzyl)-N′-(4-isopropoxybenzyl)carbamide oxalate; (410) N-[1-(2,2-Dimethyl-1,3-dioxan-5-yl)-piperidin-4-yl]-N-(4-fluorobenzyl)-N′-(4-isopropoxybenzyl)carbamide oxalate; (411) N-(4-Fluorobenzyl)-N′-(4-isopropoxybenzyl)-N-{[2-(1-methyl pyrrolidin-2-yl)ethyl]-piperidin-4-yl}carbamide oxalate; (412) N-[1-(2,2-Dimethyl-1,3-dioxan-5-yl)piperidin-4-yl]-N-(4-fluorobenzyl)-2-(4-isobutoxyphenyl)acetamide oxalate; (413) N-[1-(1,3-Dioxan-5-yl)-piperidin-4-yl)-N-(4-fluorobenzyl)-2-(4-isobutoxyphenyl)acetamide tartrate; (414) N-[1-(2,2-Dimethyl-1,3-dioxan-5-yl)piperidin-4-yl]-N-(4-fluorobenzyl)-2-(4-fluorophenyl)acetamide tartrate; (415) N-{1-[2-(1,3-Dioxan-4-yl)ethyl]piperidin-4-yl}-N-(4-fluorobenzyl)-2-(4-fluorophenyl)acetamide tartrate: (416) N-{1-[2-(1,3-Dioxan-4-yl)ethyl]piperidin-4-yl}-N-(4-fluorobenzyl)-2-(4-trifluoromethoxyphenyl)acetamide tartrate: (417) N-{1-[2-(1,3-Dioxan-4-yl)ethyl]piperidin-4-yl}-N-(4-fluorobenzyl)-2-(4-propoxyphenyl)acetamide tartrate; (418) N-(4-Fluorobenzyl)-2-(4-isobutoxyphenyl)-N-[1-(tetrahydropyran-4-yl)piperidin-4-yl]acetamide tartrate; (419) N-(4-Fluorobenzyl)-2-(4-isobutoxyphenyl)-N-[1-(tetrahydropyran-4-ylmethyl)piperidin-4-yl]acetamide tartrate; (420) N-(4-Fluorobenzyl)-2-(4-isobutoxyphenyl)-N-{1-[2-(tetrahydropyran-4-yl)ethyl]piperidin-4-yl]acetamide tartrate; (421) N-(4-Fluorobenzyl)-2-(4-fluorophenyl)-N-[1-(tetrahydropyran-4-yl)piperidin-4-yl]acetamide tartrate; (422) N-[1-((S)-3,5-Dihydroxypentyl)piperidine-4-yl]-N-(4-fluorobenzyl)-2-(4-isobutoxyphenyl)acetamide tartrate; (423) N-{1-[2-((4S)-1,3-Dioxane-4-yl)ethyl]piperidine-4-yl}-N-(4-fluorobenzyl)-2(4-isobutoxyphenyl)acetamide tartrate; (424) N-{1-[2-(1,3-Dioxan-2-yl)ethyl]piperidin-4-yl}-N-(4-fluorobenzyl)amine; (425) 2-(4-Benzyloxyphenyl)-N-{1-[2-(1,3-dioxan-2-yl)ethyl]piperidin-4-yl}-N-(4-fluorobenzyl)acetamide tartrate; (426) N-{1-[2-(1,3-Dioxan-2-yl)ethyl]piperidin-4-yl}-N-(4-fluorobenzyl)-2-(4-hydroxyphenyl)-acetamide tartrate; (427) N-{1-[2-(1,3-Dioxan-2-yl)ethyl]piperidin-4-yl}-N-(4-fluorobenzyl)-2-(4-methoxyphenyl)-acetamide tartrate; (428) N-{1-[2-(1,3-Dioxan-2-yl)ethyl]piperidin-4-yl}-N-(4-fluorobenzyl)-2-(4-isopropylphenyl)-acetamide tartrate; (429) N-{1-[2-(1,3-Dioxan-2-yl)ethyl]piperidin-4-yl}-N-(4-fluorobenzyl)-2-(4-trifluoromethoxy-phenypacetamide tartrate; (430) N-{1-[2-(1,3-Dioxan-2-yl)ethyl]-piperidin-4-yl}-N-(4-fluorobenzyl)-2-(4-ethoxyphenyl)-acetamide oxalate; (431) N-{1-[2-(1,3-Dioxan-2-yl)ethyl]piperidin-4-yl}-N-(4-fluorobenzyl)-2-(4-isopropoxyphenyl)-acetamide oxalate; (432) N-{1-[2-(1,3-Dioxan-2-yl)ethyl]piperidin-4-yl}-N-(4-fluorobenzyl)-2-phenylacetamide oxalate; (433) N-{1-[2-(1,3-Dioxan-2-yl)ethyl]piperidin-4-yl}-N-(4-fluorobenzyl)-2-[4-(2-fluoroethoxy)-phenyl]acetamide oxalate; (434) N-{1-[2-(5,5-Dimethyl-1,3-dioxan-2-yl)ethyl]piperidin-4-yl}-N-(4-fluorobenzyl)-2-(4-isobutoxyphenyl)acetamide oxalate; (435) N-(4-Fluorobenzyl)-2-(4-isobutoxyphenyl)-N-{1-[2-((R)-4-methyl-1,3-dioxan-2-yl)ethyl]-piperidin-4-yl}acetamide oxalate; (436) N-(4-Fluorobenzyl)-2-(4-isobutoxyphenyl)-N-{1-[2-((S)-4-methyl-1,3-dioxolan-2-yl)ethyl]piperidin-4-yl}acetamide oxalate; (437) N-{1-[2-(4,6-Dimethyl-1,3-dioxan-2-yl)ethyl]piperidin-4-yl}-N-(4-fluorobenzyl)-2-(4-isobutoxyphenyl)acetamide oxalate; (438) N-(4-Fluorobenzyl)-N-{1-[2-((S)-4-methyl-1,3-dioxolan-2-yl)ethyl]piperidin-4-yl}-2-(4-trifluoromethoxyphenyl)acetamide oxalate; (439) N-(4-Fluorobenzyl)-2-(4-isopropylphenyl)-N-{1-[2-((S)-4-methyl-1,3-dioxolan-2-yl)ethyl]-piperidin-4-yl}acetamide oxalate; (440) N-(4-Fluorobenzyl)-N-{1-[2-((R)-4-methyl-1,3-dioxan-2-yl)ethyl]piperidin-4-yl}-2-(4-trifluoromethoxyphenyl)acetamide oxalate; (441) N-(4-Fluorobenzyl)-2-(4-isobutoxyphenyl)-N-{1-[2-(2,5,5-trimethyl-1,3-dioxan-2-yl)ethyl]piperidin-4-yl}acetamide oxalate; (442) N-(4-Fluorobenzyl)-2-(4-isobutoxyphenyl)-N-{1-[2-(2-methyl-1,3-dioxolan-2-yl)ethyl]-piperidin-4-yl-}acetamide oxalate; (443) N-(4-Fluorobenzyl)-2-(4-isobutoxyphenyl)-N-{1-[3-(1,3-dioxolan-2-yl)propyl]piperidin-4-yl}acetamide tartrate; (444) N-(4-Fluorobenzyl)-2-(4-isobutoxyphenyl)-N-{1-(3-piperidin-1-yl-propyl)piperidin-4-yl}-acetamide dihydrochloride; (445) N-(4-Fluorobenzyl)-2-(4-isobutoxyphenyl)-N-{1-[2-(tetrahydropyran-2-yloxy)ethyl]-piperidin-4-yl}acetamide oxalate; (446) N-(4-Fluorobenzyl)-2-(4-isobutoxyphenyl)-N-{1-[3-(2-oxo-piperidin-1-yl)propyl]piperidin-4-yl}acetamide; (447) N-(4-Fluorobenzyl)-2-(4-isobutoxyphenyl)-N-{1-[3-(2-oxo-pyrrolidin-1-yl)propyl]piperidin-4-yl}acetamide hydrochloride; (448) N-(4-Fluorobenzyl)-2-(4-isobutoxyphenyl)-N-{1-[3-((R)-4-isopropyl-2-oxo-oxazolidin-3-yl)propyl]piperidin-4-yl}acetamide oxalate; (449) N-(4-Fluorobenzyl)-2-(4-isobutoxyphenyl)-N-{1-[3-(2-oxo-oxazolidin-3-yl)propyl]piperidin-4-yl}acetamide oxalate; (450) N-(4-Fluorobenzyl)-2-(4-isobutoxyphenyl)-N-{1-[3-((S)-4-methyl 2-oxo-oxazolidin-3-yl)propyl]piperidin-4-yl}acetamide tartrate; (451) N-(4-Fluorobenzyl)-2-(4-isobutoxyphenyl)-N-{1-[3-((S)-4-ethyl-2-oxo-oxazolidin-3-yl)-propyl]piperidin-4-yl}acetamide oxalate; (452) N-(4-Fluorobenzyl)-2-(4-isobutoxyphenyl)-N-{1-[2-(1,3-oxothiolan-2-yl)ethyl]piperidin-4-yl}acetamide L-tartrate; (453) 2-(4-Bromophenyl)-N-{1-[2-(1,3-dioxan-2-yl)ethyl)piperidin-4-yl}-N-(4-fluorobenzyl)-acetamide L-tartrate; (454) N-{1-[2-(1,3-Dioxan-2-yl)ethyl)piperidin-4-yl}-N-(4-fluorobenzyl)-2-(4-isobutylamino-phenyl)acetamide L-tartrate; (455) N-{1-[2-(1,3-Dioxan-2-yl)ethyl)piperidin-4-yl}-N-(4-fluorobenzyl)-2-(4-propylaminophenyl)acetamide L-tartrate; (456) N-{1-[2-(1,3-Dioxan-2-yl)ethyl)piperidin-4-yl}-N-(4-fluorobenzyl)-2-(4-(1-nitropropyl)-phenyl)acetamide L-tartrate; (457) N-{1-[2(1,3-Dioxan-2-yl)ethyl)piperidin-4-yl}-N-(4-fluorobenzyl)-2-[4-(2-oxopyrrolidin-1-yl)phenyl)acetamide L-tartrate; (458) N-{1-[2-(1,3-Dioxan-2-yl)ethyl)piperidin-4-yl}-N-(4-fluorobenzyl)-2-(4-isobutylsulfanylphenyl)acetamide L-tartrate; (459) N-{1-[2-(1,3-Dioxan-2-yl)ethyl)piperidin-4-yl}-N-(4-fluorobenzyl)-2-(4-iodophenyl)-acetamide L-tartrate; (460) 2-(4-Acetophenyl)-N-{1-[2-(1,3-dioxan-2-yl)ethyl)piperidin-4-yl}-N-(4-fluorobenzyl)-acetamide L-tartrate; (461) 2-[4-(1-hydroxyiminoethyl)phenyl]-N-{1-[2-(1,3-dioxan-2-yl)ethyl)piperidin-4-yl}-N-(4-fluorobenzyl)acetamide L-tartrate; (462) N-{1-[2-(1,3-Dioxan-2-yl)ethyl)piperidin-4-yl}-N-(4-fluorobenzyl)-2-(4-morpholin-4-yl-phenyl)acetamide L-tartrate; (463) N-{1-[2-(1,3-Dioxan-2-yl)ethylypiperidin-4-yl}-N-(4-fluorobenzyl)-2-(4-pyrazol-1-ylphenyl)acetamide L-tartrate; (464) N-{1-[2-(1,3-Dioxan-2-yl)-1-methylethyl]piperidin-4-yl}-N-(4-fluorobenzyl)-2-(4-isobutoxyphenyl)-acetamide L-tartrate; (465) N-{1-[2-(1,3-Dioxan-4-yl)ethyl)piperidin-4-yl}-N-(4-fluorobenzyl)-2-(4-pyrazol-1-ylphenyl)acetamide L-tartrate; (466) N-[1-((R)-3,5-Dihydroxypentyl)pipe-ridine-4-yl]-N-(4-fluorobenzyl)-2-(4-isobutoxyphenyl)acetamide tartrate; (467) N-{1-[2-((4R)-1,3-Dioxane-4-yl)ethyl]piperidine-4-yl}-N-(4-fluorobenzyl)-2-(4-isobutoxyphenyl)acetamide tartrate; (468) N-{1-[2-(1,3-Dioxan-2-yl)ethyl]piperidin-4-yl}-N-(4-fluorobenzyl)-2-[4-(1,2,4-triazol-4-yl)phenyl]acetamide L-tartrate; (469) nortriptyline; (470) duloxetine; (471) lofepramine; (472) tomoxetine; (473) 3-({1-[2-(7-methyl-5-oxo-5H)-[1,3]thiazolo[3,2-a]pyrimidin-6-yl)ethyl]-3-pyrrolidinyl}methyl)-1H-indole-5-carbonitrile hydrochloride; (474) 3-({1-[2-(6-chloro-2-oxo-2,3-dihydro-1H-indol-5-yl)ethyl]-3-pyrrolidinyl}-methyl)-1H-indole-5-carbonitrile hydrochloride; (475) moclobemide; (476) N-acetylserotonin; (477) bromfaromine; (478) beflaxozone; (479) chlorimipramine; (480) cyanimipramine; (481) cianopramine; (482) desipramine; (483) protriptyline; (484) trimipramine; (485) doxepin; (486) cyclobenzaprine; (487) 5-methoxycarbonylamino-N-acetyltryptamine; (488) amoxapine; (489) maprotiline; (490) fefazodone; (491) flesinoxan hydrochloride; (492) urapidil; (493) WY47846 (3a,4,4a,6a,7,7a-hexahydro-2-[4-[4-(2-pyrimidinyl)-1-piperazinyl]-butyl]-4,7-etheno-1H-cyclobutano[f]isoindole-1,3(2H)-dione dihydrochloride sesquihydrate); (494) SM3997 (N-[4-[4-(2-pyrimidinyl)-1-piperazinyl]butyl]-bicyclo[2.2.1]heptane-2,3-di-exo-carboximide); (495) 2-(4-(4-(2-pyrimidinyl)-1-piperazinyl-propyl)-1,2-benzoisothiazol-3-(2H)-one 1,1-dioxide hydrochloride; (496) KC9172 (3-butyl-9,9-dimethyl-7-[4-[4-[2-methoxyphenyl)-1-piperazinyl]butyl]-3,7-diazabicyclo[3,2,1]nonane-2,4,6,8-tetraone); (497) 4-(N,N-dipropylamino)-6-methoxy-1,3,4,5-tetrahydrobenzqc,d]indole; (498) 4-[4-(N-1,2-benzisothiazol-3(2H)-one 1,1-dioxido)]butylamino-6-methoxy-1,3,4,5-tetrahydrobenz[c,d]-indole hydrochloride; (499) 5-carboxamidotryptamine; (500) N,N-dipropyl-5-carboxamidotryptamine; (501) AH25086 (3-(2-aminoethyl)-1H-indole-5-(N-methyl)acetamide); (502) GR43175 (3-(2-dimethylaminoethyl)-1H-indole-5-(N-methyl)methanesulfonamide); (503) 3-(2-[4-[2-(1,2-benzisothiazole-3(2H)-one 1,1-dioxido)]butyl]amino)ethyl-5-methoxy-1H-indole; (504) spiroxatrine; (505) MDL72832 (8-[4-(1,4-benzodioxan-2-ylmethylamino)butyl]-8-azaspiro-[4,5]decane-7,9-dione); (506) 2-[4-(1,4-benzodioxan-2-ylmethylamino)butyl]-1,2-benzisothiazol-3(2H)-one 1,1-dioxide; (507) 2-(N,N-dipropylamino)-8-hydroxy-1,2,3,4-tetrahydronaphthalene; (508) 2-{4-[2-(1,2-benzisothiazol-3(2H)-one 1,1-dioxido)]butyl}amino-8-methoxy-1,2,3,4-tetrahydronaphthalene; (509) 3-N,N-dipropylamino-5-hydroxy-thiochroman; 3-N,N-dipropylamino-5-ethoxy-thiochroman; (510) 3-N,N-dipropylamino-5-ethoxychroman; (511) 1-[2-(3-indolyl)]-ethyl-2,6-dimethylpiperidine; (512) 1-{2-[3-(5-carboxamido)indolyl]}ethyl-2,6-dimethylpiperidine; (513) RU24924 (5-methoxy-3-(1,2,3,6-tetrahydropyridin-4-yl]-1H-indole); (514) 5-methoxy-3-(1,2,3,6-tetrahydropyridin-5-yl)-1H-indole; (515) diethyl N-benzyloxycarbonyl-5-benzyloxycarbonyloxy-L-tryptophyl-L-aspartate; (516) dibenzyl N-benzyloxycarbonyl-5-hydroxy-L-tryptophanylaspartate; (517) 5-Hydroxy-L-tryptophyl-L-aspartic acid trihydrate; (518) diethyl N-benzyloxycarbonyl-5-hydroxy-L-tryptophyl-L-glutamate; (519) diethyl 5-hydroxy-L-tryptophyl-L-glutamate hydrochloride; (520) dibenzyl L-benzyloxycarbonyl-5-hydroxytryptophyl-L-glutamate; (521) 5-hydroxy-L-tryptophyl-L-glutamic acid; (522) pentachlorophenyl ester of N-benzyloxycarbonyl-5-hydroxy-L-tryptophan; (523) methyl ester of N-benzyloxycarbonyl-5-hydroxy-L-tryptophyl-L-tyrosine; (524) N-Acetyl-5-hydroxy-L-tryptophan; (525) methyl ester of N-acetyl-5-hydroxy-L-tryptophyl-L-tyrosine; (526) methyl ester of N-acetyl-5-hydroxy-L-tryptophyl-5-hydroxy-L-tryptophan; (527) 5-hydroxy-L-tryptophyl-L-alanine hydrate; (528) 5-hydroxy-L-tryptophan-L-valine; (529) 5-hydroxy-L-tryptophyl-L-leucine; (530) 5-hydroxy-L-tryptophyl-L-proline; (531) 5-hydroxy-L-tryptophyl-L-phenylalanine; (532) 5-hydroxy-L-tryptophyl-5-hydroxy-L-tryptophan; (533) 5-hydroxy-L-tryptophyl-L-tryptophan; (534) 1-(5-hydroxy)tryptophyl-L-serine; (535) 5-hydroxy-L-tryptophyl-L-arginine; (536) 5-hydroxy-L-tryptophylglycine; (537) 5-hydroxy-1-tryptophyl-gamma-aminobutyric acid; (538) 5-hydroxy-L-tryptophanamide hydrate; (539) methyl ester of 5-hydroxy-L-tryptophyl-L-histidine; (540) benzyl ester of L-5-hydroxytryptophan; (541) benzyl ester of N-benzyloxycarbonyl-5-hydroxy-L-tryptophyl-5-hydroxy-L-tryptophan; (542) 5-Hydroxy-L-tryptophyl-5-hydroxy-L-tryptophan hemihydrate; (543) 5-hydroxytryptophan inosinate; (544) theophylline salt of (DL) 5-hydroxytryptophan; (545) RU25591 (6,7,8,9-tetrahydro N, N-dimethyl 5-[4-nitrophenyl]oxy 5H-benzocyclohepten 7-amine) cis-fumarate); (546) LM5008 (4-[2-(3-indolyl)ethyl]piperidine); (547) DU24565 (6-nitro-2-(1-piperazinyl)quinoline); (548) CGP6085A (4-(5,6-dimethyl-2-benzofuranyl) piperidine hydrochloride); (549) alaprociate; (550) dibenzoxazepine; (551) deprenyl; (552) isocarboxazide; (553) furazolidone; (554) procarbazine; (555) Ro 60-0175/ORG 35030 ((S)-2-(4,4,7-trimethyl-1,4-dihydro-indeno (1,2-B) pyrrol-1-yl)-1-methyl-ethylamine) (556) Ro 60-0332/ORG 35035 ((S)-2-(Chloro-5-fluoro-indol-1-yl)-1-methylethylamine); (557) 1-[6-Chloro-5-trifluoromethyl)-2-pyridinyl]-piperazine hydrochloride; (558) 5-carboxyamidotryptamine; (559) SB 206553 (3,5-Dihydro-5-methyl-N-3-pyridinylbenzo[1,2-b:4,5-b′]dipyrrole-1(2H)-carboxamide hydrochloride); (560) ondansetron; (561) granisetron; (562) tropisetron; (563) dolasetron; (564) palonosetron; (565) trimethobenzamide; (566) risperidone; (567) clozapine; (568) azatadine; (569) cyproheptadine; (570) fenclonine; (571) chlorpromazine; (572) (313)-2,3-dihydrolysergine; (573) (313)-2,3-dihydroisolysergine; (574) (3β, 5β, 8β)-9,10-didehydro-2,3-dihydro-6-methylergoline-8-acetonitrile; (575) 25I-NBMD (2-(4-iodo-2,5-dimethoxyphenyl)-N-[(2,3-methylenedioxyphenyl)methyl]ethanamine); (576) N-(2-methoxybenzyl)-1-(8-bromo-2,3,6,7-tetrahydrobenzo[1,2-b:4,5-b′]difuran-4-yl)-2-aminoethane; (577) 5-benzyloxytryptamine; (578) 5-methoxy-7-N,N-dimethyltryptamine; (579) A372159 ((11S,16R)-3-[4-(propan-2-yloxy)-2-(trifluoromethyl)phenyl]-6-oxa-10,14-diazatetracyclo[8.6.1.0^{5,17}.0^{11,16}]heptadeca-1,3,5(17)-triene); (580) AL-34662 (1-((S)-2-Aminopropyl)-1H-indazol-6-ol); (581) AL-37350A ((S)-(+)-1-(2-Aminopropyl)-8,9-dihydropyrano[3,2-e]indole); (582) AL-38022A ((S)-2-(8,9-dihydro-7H-pyrano[2,3-g]indazol-1-yl)-1-methylethylamine); (583) AS-19 ((2S)—N,N-dimethyl-5-(1,3,5-trimethylpyrazol-4-yl)-1,2,3,4-tetrahydronaphthalen-2-amine); (584) alnespirone; (585) BIMU8 (N-[(1R,5S)-8-methyl-8-azabicyclo[3.2.1]oct-3-yl]-2-oxo-3-(propan-2-yl)-2,3-dihydro-1H-benzimidazole-1-carboxamide hydrochloride); (586) BMY-14802 (1-(4-fluorophenyl)-4-[4-(5-fluoropyrimidin-2-yl)piperazin-1-yl]butan-1-ol); (587) BRL-54443 (3-(1-methylpiperidin-4-yl)-1H-indol-5-ol); (588) batoprazine; (589) benzylpiperazine; (590) binospirone; (591) 1-(8-bromobenzo[1,2-b;4,5-b]difuran-4-yl)-2-aminopropane); (592) CP-809,101 (2-[(3-Chlorophenyl)methoxy]-6-(1-piperazinyl)pyrazine); (593) CP-93,129 (3-(1,2,3,6-tetrahydropyrid in-4-yl)-1,4-d ihydropyrrolo[3,2-b]pyridin-5-one); (594) CP-94,253 (3-(1,2,5,6-tetrahydro-4-pyridyl)-5-propoxypyrrolo[3,2-b]pyridine); (595) CGS-12066A (4-(4-methylpiperazin-1-yl)-7-(trifluoromethyl)pyrrolo[1,2-a]quinoxaline); (596) chlorophenylbiguanide; (597) chlorphentermine; (598) dazopride; (599) dimemebfe; (600) 2,5-dimethoxy-4-bromoamphetamine; (601) 2,5-dimethoxy-4-fluoroamphetamine; (602) 2,5-dimethoxy-4-methylamphetamine; (603) EMD-386,088 (5-chloro-2-methyl-3-(1,2,3,6-tetrahydro-4-pyridinyl)-1H-indole); (604) EMDT (2-(2-ethyl-5-methoxy-1-indol-3-yl)-N,N-dimethylethanamine); (605) p-fluoropiperazine; (606) fluprazine; (607) jimscaline; (608) LY-293,284 ((4R)-6-acetyl-4-(di-n-propylamino)-1,3,4,5-tetrahydrobenz[c,d]indole); (609) lasmitidan; (610) lorcaserin; (611) 2-methyl-5-hydroxytryptamine; (612) 2-methyl-4,5-methylenedioxyamphetamine; (613) NBUMP (N-[4-[4-(2-methoxyphenyl)piperazin-1-yl]butyl]adamantane-1-carboxamide); (614) 1-(1-naphthyl)piperazine; (615) Org-37,684 ((3S)-3-[(2,3-dihydro-5-methoxy-1H-inden-4-yl)oxy]pyrrolidine); (616) PNU-22394 (6-Methyl-1,2,3,4,5,6-hexahydro-azepino[4,5-b]indole)); (617) PRX-00023 (N-(3-[4-(4-cyclohexylmethanesulfonylaminobutyl)piperazin-1-yl]phenyl)acetamide); (618) RH-34 (3-[2-(2-methoxybenzylamino)ethyl]-1H-quinazoline-2,4-dione); (619) RS56812 (N-(3R)-1-azabicyclo[2.2.2]oct-3-yl]-2-(1-methyl-1H-indol-3-yl)-2-oxoacetamide); (620) RS67333 (1-(4-amino-5-chloro-2-methoxyphenyl)-3-(1-butyl-4-piperidinyl)-1-propanone); (621) RU24969 (5-Methoxy-3-(1,2,5,6-tetrahydro-4-pyridinyl)-1H-indole); (622) Ro60-0175 ((S)-6-Chloro-5-fluoro-1H-indole-2-propanamine); (623) TFMFIy ((2R)-1-(8-trifluoromethyl-2,3,6,7-tetrahydrobenzo[1,2-b:4,5-b′]difuran-4-yl)-2-aminoethane); (624) U92016-A ((8R)-8-(Dipropylamino)-6,7,8,9-tetrahydro-3H-benz[e]indole-2-carbonitrile) (625) VER3323 ((2S)-1-(6-bromo-2,3-dihydroindol-1-yl)propan-2-amine); (626) vilazodone; (627) WAY-181,187 (1-[(2S,5S)-4,4-difluoro-5-(hydroxymethyl)tetrahydrofuran-2-yl]pyrimidine-2,4(1H,3H)-dione); (628) WAY-208,466 (N′-[(2Z)-4-(2,4-dichlorophenyl)-3-(2-methylpropyl)-1,3-thiazol-2(3H)-ylidene]-2-(pyrazin-2-yloxy)acetohydrazide); (629) YM-348 (2S)-1-(7-ethyl-1H-furo[2,3-g]indazol-1-yl)propan-2-amine); (630) alprenolol; (631) BMY 7378 (8-(2-[4-(2-methoxyphenyl)-1-piperazinyl]ethyl)-8-azaspiro[4.5]decane-7,9-dione); (632) cyanopindolol; (633) iodocyanopindolol; (634) lezcotozan; (635) methiothepin; (636) NAN-190 (1-(2-methoxyphenyl)-4-(4-phthalimidobutyl)piperazine); (637) oxprenolol; (638) pindolol; (639) propranolol; (640) robalzotan; (641) S15535 (1-(2,3-dihydro-1,4-benzodioxin-8-yl)-4-(2,3-dihydro-1H-inden-2-yl)piperazine); (642) spiperone; (643) TFMPP; (644) UH-301 ((S)-5-fluoro-8-hydroxy-2-(dipropylamino)tetralin); (645) WAY-100,135 ((S)—N-tert-butyl-3-(4-(2-methoxyphenyl)-piperazin-1-yl)-2-phenylpropanamide); (646) WAY-100,635 (N-[2-[4-(2-methoxyphenyl)-1-piperazinyl]ethyl]-N-(2-pyridyl)cyclohexanecarboxamide); (647) mefway; (648) 5-hydroxytryptophan; (649) 5-hydroxytryptophan creatinine sulfate complex; (650) 5-methoxytryptamine; (651) 5-methoxytryptamine creatinine sulfate complex; (652) 5-HIAA (5-hydroxyindoleacetic acid); and (653) 5-HIAA (5-hydroxyindoleacetic acid) creatinine sulfate complex; and the salts, solvates, analogues, congeners, bioisosteres, hydrolysis products, metabolites, precursors, and prodrugs thereof.

The composition can further comprise a pharmaceutically acceptable carrier. Suitable pharmaceutically acceptable carriers are described below.

In one alternative, the composition consists essentially of the first and second agents, or, if a pharmaceutically acceptable carrier is included, of the first and second agents and the pharmaceutically acceptable carrier. In this alternative, the composition is limited to the specified materials and those that do not materially affect the basic and novel characteristics of the composition.

In one alternative, the first agent is associated with a carrier substance to facilitate the transport of the first agent to an intended site of action of the first agent. The carrier substance can be, but is not limited to, an antibody, an antibody fragment, or a receptor. The first agent can be covalently or noncovalently bound to the carrier substance.

In another alternative, the second agent is associated with a carrier substance to facilitate the transport of the second agent to an intended site of action of the second agent. The carrier substance can be, but is not limited to, an antibody, an antibody fragment, or a receptor. The second agent can be covalently or noncovalently bound to the carrier substance.

In yet another alternative, the first agent and the second agent are each associated with a carrier substance to facilitate the transport of the first agent and the second agent to intended sites of action of the first agent and the second agent. The first agent and the second agent can be each associated with a single carrier substance, such as an antibody, an antibody fragment, or a receptor. Alternatively, the first agent and the second agent can be associated with separate carrier substances. The first agent and the second agent can be covalently or noncovalently bound to the carrier substance or carrier substances.

Methods for binding the first agent or second agent to an individual carrier substance are known in the art. Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the 8-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail. Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

The individual carrier substances can be, but are not limited to, antibodies, hormones, receptor agonists or antagonists, or receptors. As used herein, unless further defined or limited, the term “antibody” encompasses both polyclonal and monoclonal antibodies, as well as genetically engineered antibodies such as chimeric or humanized antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. Receptors are well known in the art and include G-protein coupled receptors (GPCRs). G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors. Agonists and antagonists specifically binding these receptors can be used as individual carrier substances; suitable receptors, agonists, or antagonists can be selected based on their specificity and the location of the receptors in particular cells or tissues.

Typically, the composition comprises from about 0.1 mg to about 10 g of the first agent per unit dose and from about 0.1 mg to about 10 g of the second agent per unit dose. In one alternative, the composition comprises about 0.1 mg of the first agent per unit dose and about 0.1 mg of the second agent per unit dose. In another alternative, the composition comprises about 5 g of the first agent per unit dose and about 5 g of the second agent per unit dose. In still another alternative, the composition comprises about 10 g of the first agent per unit dose and about 10 g of the second agent per unit dose. For example, the composition can comprise from about 0.1 g to about 10 g of a first agent selected from the group consisting of metformin, phenformin, buformin, AICAR, thienopyridones, resveratrol, nootkatone, thiazole, adiponectin, thiazolidinediones, rosiglitazone, pioglitazone, dithiolethiones, and the salts, solvates, analogues, congeners, bioisosteres, hydrolysis products, metabolites, precursors, and prodrugs thereof and from about 0.1 mg to about 10 g of a second agent selected from the group consisting of serotonin sulfate, serotonin creatinine sulfate complex, serotonin hydrochloride, melatonin, 5-hydroyxindoleacetic acid, a salt of 5-hydroxyindoleacetic acid, melatonin creatinine sulfate complex, and 5-hydroxyindoleacetic acid creatinine sulfate complex. As another example, the composition can comprise from about 3 g to about 10 g of metformin hydrochloride per unit dose and from about 3 g to about 10 g of melatonin per unit dose.

Particularly, the composition comprises the first agent and the second agent in a weight ratio of 1-1000:0.01-1. More particularly, the composition comprises the first agent and the second agent in a weight ratio of 1-100:0.05-1. Even more particularly, the composition comprises the first agent and the second agent in a weight ratio of 10-100:0.1-1. In one example, the composition comprises the first and second agent in a weight ratio of about 150:1.

Another aspect of the present invention is a method of treating a disease or condition comprising the step of administering a therapeutically effective quantity of a pharmaceutical composition according to the present invention as described above to a subject that has the disease or condition or that is at risk of developing the disease or condition, in order to treat or prevent the occurrence of the disease or condition, wherein the disease or condition is selected from the group consisting of metabolic syndrome, diabetes, obesity, hypertension, cancer, AIDS, Parkinson's disease, polycystic ovarian syndrome, Alzheimer's disease, osteoporosis, sleep apnea, erectile dysfunction, McArdle disease, and a carbohydrate metabolism disorder. Typically, the disease or condition is selected from the group consisting of metabolic syndrome, diabetes, obesity, and hypertension. In another alternative, the disease or condition is cancer. In still another alternative, the disease or condition is selected from the group consisting of Parkinson's disease, polycystic ovarian syndrome, Alzheimer's disease, osteoporosis, sleep apnea, erectile dysfunction, McArdle disease, and a carbohydrate metabolism disorder.

The pharmaceutical composition can be administered orally or parenterally. Parenteral administration includes, but is not limited to, a route of administration selected from the group consisting of subcutaneous, intracutaneous, intravenous, intramuscular, intraarticular, intraarterial, intrasynovial, intrasternal, intrathecal, intralesional, and intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition for topical administration can be prepared in form of an ointment, a gel, a plaster, an emulsion, a lotion, a foam, a cream of a mixed phase or amphiphilic emulsion system (oilwater-wateroil mixed phase), a liposome, a transfersome, a paste, or a powder.

Any of the compositions described above can also be administered in the form of suppositories for rectal administration. It also can be designed such that the composition is released in the intestine. For example, the composition is confined in a solid sub-unit or a capsule compartment that have respectively a matrix or a wall or a closure comprising an enteric polymer which dissolves or disperses at the pH of the small or large intestine to release the drug substance in the intestine. Suitable such polymers have been described above, for example with reference to U.S. Pat. No. 5,705,189.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active thiophene compound. Examples of other carriers include colloidal silicon oxide, magnesium stearate, cellulose, sodium lauryl sulfate, and D&C Yellow #10.

The compositions described above can be used to treat diseases and conditions such as metabolic syndrome, Parkinson's disease, or polycystic ovarian syndrome. The diseases mentioned above also include their associated disorders. For example, disorders associated with metabolic syndrome include atherosclerosis, coronary heart disease, stroke, obesity, diabetes, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels).

The compositions described above can also be used to treat additional diseases and conditions, including hyperproliferative diseases and Alzheimer's disease. Hyperproliferative diseases include benign tumors and malignant tumors, as well as non-tumor hyperproliferative diseases. Benign tumors include, but are not limited to: adrenal tumors such as adenoma, adrenal pheochromocytoma and adrenal ganglioneuroma; brain tumors such as meningioma and adenoma; peripheral nerve tumors such as neurofibroma and schwannoma; liver tumors such as adenoma; thyroid tumors such as follicular adenoma; parathyroid tumors such as adenoma; thymus tumors such as thymoma; salivary gland tumors such as pleomorphic adenoma; small intestine tumors such as villous adenoma; colon tumors such as tubulovillous adenoma, adenomatous polyp of colon, and polyposis coli; pancreas tumors such as serous cystadenoma; islet tumors such as pancreatic islet cell tumor; nasopharyngeal tumors such as nasal angiofibroma; ovarian tumors such as atypical proliferating mucinous neoplasm, Brenner tumor of ovary, mucinous cystadenoma, papillary cystadenoma, dermoid cyst of ovary, ovarian teratoma, ovarian fibroma, luteoma, and struma ovarii; uterine tumors such as uterine cellular leiomyoma and leiomyoma; placental tumors such as chorioangioma, partial hydatidiform mole, and complete hydatidiform mole; bone tumors such as cavernous hemangioma and giant cell tumor; soft tissue tumors such as cavernous hemangioma, desmoid tumor, lipoma, myelolipoma, and osteochondroma; joint tumors such as synovial chondromatosis; lung tumors such as carcinoid tumor, granular cell tumor, and hemangioma; myocardium tumors such as atrial myxoma; breast tumors such as fibroadenoma, intraductal papilloma and schwannoma; kidney tumors such as congenital mesoblastic nephroma; and skin tumors such as giant congenital intradermal nevus.

As used generally herein, the term “hyperproliferative disorders” refers to excess cell proliferation that is not governed by the usual limitation of normal growth. The term denotes malignant as well as nonmalignant cell populations. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Hyperproliferative cell disorders include tumors as well as non-tumor conditions. A “tumor” here refers to an abnormal mass of tissue that results from excessive cell division that is uncontrolled and progressive, also called a neoplasm.

Examples of tumors include a variety of solid tumors such as laryngeal tumors, brain tumors, other tumors of the head and neck; colon, rectal and prostate tumors; breast and thoracic solid tumors; ovarian and uterine tumors; tumors of the esophagus, stomach, pancreas, and liver; bladder and gall bladder tumors; skin tumors such as melanomas and the like; and a fluid tumor such as leukemia.

A “solid tumor,” as used herein, refers to an abnormal mass of tissue that usually does not contain cysts or liquid areas. Solid tumors may be benign (not cancerous) or malignant (cancerous). Solid tumors have a distinct structure that mimics that of normal tissues and comprises two distinct but interdependent compartments: the parenchyma (neoplastic cells) and the stroma that the neoplastic cells induce and in which they are dispersed. Different types of solid tumors are named for the type of cells that form them. Examples of solid tumors are sarcomas, carcinomas, and lymphomas. Solid tumors are loci of tumor cells in which the majority of cells are tumor cells or tumor-associated cells.

More particularly, “tumor” as used herein refers to either benign (non-cancerous) or malignant tumors.

Malignant tumors include, but are not necessarily limited to: (A) breast cancer, including: (1) ductal carcinoma, including ductal carcinoma in situ (DCIS) (comedocarcinoma, cribriform, papillary, micropapillary), infiltrating ductal carcinoma (IDC), tubular carcinoma, mucinous (colloid) carcinoma, papillary carcinoma, metaplastic carcinoma, and inflammatory carcinoma; (2) lobular carcinoma, including lobular carcinoma in situ (LCIS) and invasive lobular carcinoma; and (3) Paget's disease of the nipple; (B) cancers of the female reproductive system, including: (1) cancers of the cervix uteri, including cervical intraepithelial neoplasia (Grade I), cervical intraepithelial neoplasia (Grade II), cervical intraepithelial neoplasia (Grade III) (squamous cell carcinoma in situ), keratinizing squamous cell carcinoma, nonkeratinizing squamous cell carcinoma, verrucous carcinoma, adenocarcinoma in situ, adenocarcinoma in situ, endocervical type, endometrioid adenocarcinoma, clear cell adenocarcinoma, adenosquamous carcinoma, adenoid cystic carcinoma, small cell carcinoma, and undifferentiated carcinoma; (2) cancers of the corpus uteri, including endometrioid carcinoma, adenocarcinoma, adenocanthoma (adenocarcinoma with squamous metaplasia), adenosquamous carcinoma (mixed adenocarcinoma and squamous cell carcinoma, mucinous adenocarcinoma, serous adenocarcinoma, clear cell adenocarcinoma, squamous cell adenocarcinoma, and undifferentiated adenocarcinoma; (3) cancers of the ovary, including serous cystadenoma. serous cystadenocarcinoma, mucinous cystadenoma, mucinous cystadenocarcinoma, endometrioid tumor, endometrioid adenocarcinoma, clear cell tumor, clear cell cystadenocarcinoma, and unclassified tumor; (4) cancers of the vagina, including squamous cell carcinoma and adenocarcinoma; and (5) cancers of the vulva, including vulvar intraepithelial neoplasia (Grade I), vulvar intraepithelial neoplasia (Grade II), vulvar intraepithelial neoplasia (Grade III) (squamous cell carcinoma in situ); squamous cell carcinoma, verrucous carcinoma, Paget's disease of the vulva, adenocarcinoma (NOS), basal cell carcinoma (NOS), and Bartholin's gland carcinoma; (C) cancers of the male reproductive system, including: (1) cancers of the penis, including squamous cell carcinoma; (2) cancers of the prostate, including adenocarcinoma, sarcoma, and transitional cell carcinoma of the prostate; (3) cancers of the testis, including seminomatous tumor, nonseminomatous tumor, teratoma, embryonal carcinoma, yolk sac tumor, and choriocarcinoma; (D) cancers of the cardiac system, including sarcoma (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma, and squamous cell carcinoma of the pharynx; (F) cancers of the lung, including squamous cell carcinoma (epidermoid carcinoma), variants of squamous cell carcinoma, spindle cell carcinoma, small cell carcinoma, carcinoma of other cells, carcinoma of intermediate cell type, combined oat cell carcinoma, adenocarcinoma, acinar adenocarcinoma, papillary adenocarcinoma, bronchiolo-alveolar carcinoma, solid carcinoma with mucus formation, large cell carcinoma, giant cell carcinoma, clear cell carcinoma, and sarcoma; (G) cancers of the gastrointestinal tract, including: (1) cancers of the ampulla of Vater, including primary adenocarcinoma, carcinoid tumor, and lymphoma; (2) cancers of the anal canal, including adenocarcinoma, squamous cell carcinoma, and melanoma; (3) cancers of the extrahepatic bile ducts, including carcinoma in situ, adenocarcinoma, papillary adenocarcinoma, adenocarcinoma, intestinal type, mucinous adenocarcinoma, clear cell adenocarcinom, segnet-ring cell carcinoma, adenosquamous carcinoma, squamous cell carcinoma, small cell (oat) carcinoma, undifferentiated carcinoma, carcinoma (NOS), sarcoma, and carcinoid tumor; (4) cancers of the colon and rectum, including adenocarcinoma in situ, adenocarcinoma, mucinous adenocarcinoma (colloid type; greater than 50% mucinous carcinoma), signet ring cell carcinoma (greater than 50% signet ring cell), squamous cell (epidermoid) carcinoma, adenosquamous carcinoma, small cell (oat cell) carcinoma, undifferentiated carcinoma, carcinoma (NOS), sarcoma, lymphoma, and carcinoid tumor; (5) cancers of the esophagus, including squamous cell carcinoma, adenocarcinoma, leiomyosarcoma, and lymphoma; (6) cancers of the gallbladder, including adenocarcinoma, adenocarcinoma, intestinal type, adenosquamous carcinoma, carcinoma in situ, carcinoma (NOS), clear cell adenocarcinoma, mucinous adenocarcinoma, papillary adenocarcinoma, signet-ring cell carcinoma, small cell (oat cell) carcinoma, squamous cell carcinoma, and undifferentiated carcinoma; (7) cancers of the lip and oral cavity, including squamous cell carcinoma; (8) cancers of the liver, including hepatoma (hepatocellular carcinoma), cholangiocarcinoma, hepatoblastoma, angiosarcoma, hepatocellular adenoma, and hemangioma; (9) cancers of the exocrine pancreas, including duct cell carcinoma, pleomorphic giant cell carcinoma, giant cell carcinoma, osteoclastoid type, adenocarcinoma, adenosquamous carcinoma, mucinous (colloid) carcinoma, cystadenocarcinoma, acinar cell carcinoma, papillary carcinoma, small cell (oat cell) carcinoma, mixed cell typed, carcinoma (NOS), undifferentiated carcinoma, endocrine cell tumors arising in the islets of langerhans, and carcinoid; (10) cancers of the salivary glands, including acinic (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well differentiated or low grade), and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenosquamous carcinoma, squamous cell carcinoma, small cell carcinoma, undifferentiated carcinoma, lymphoma, sarcoma, and carcinoid tumor; and (12) cancers of the small intestine, including adenocarcinoma, lymphoma, carcinoid tumors, Kaposi's sarcoma, leiomyoma, hemangioma, lipoma, neurofibroma, and fibroma; (H) cancers of the urinary system, including: (1) cancers of the kidney, including renal cell carcinoma, carcinoma of Bellini's collecting ducts, adenocarcinoma, papillary carcinoma, tubular carcinoma, granular cell carcinoma, clear cell carcinoma (hypernephroma), sarcoma of the kidney, and nephroblastoma; (2) cancers of the renal pelvis and ureter, including transitional cell carcinoma, papillary transitional cell carcinoma, squamous cell carcinoma, and adenocarcinoma; (3) cancers of the urethra, including transitional cell carcinoma, squamous cell carcinoma, and adenocarcinoma; and (4) cancers of the urinary bladder, including carcinoma in situ, transitional urothelial cell carcinoma, papillary transitional cell carcinoma, squamous cell carcinoma, adenocarcinoma, undifferentiated; (I) cancers of muscle, bone, and soft tissue, including: (1) cancers of bone, including: (a) bone-forming: osteosarcoma; (b) cartilage-forming: chondrosarcoma and mesenchymal chondrosarcoma; (c) diant cell tumor, malignant; (d) Ewing's sarcoma; (e) vascular tumors: hemangioendothelioma, hemangiopericytoma, and angiosarcoma; (f) connective tissue tumors: fibrosarcoma, liposarcoma, malignant mesenchymoma, and undifferentiated sarcoma; and (g) other tumors: chordoma and adamantinoma of long bones; (2) cancers of soft tissues, including: alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, rhabdomyosarcoma, synovial sarcoma, and sarcoma (NOS); (3) cancers of the nervous system, including cancers of the skull (osteoma, hemangioma, granuloma, xanthoma, osteitis deformans), cancers of the meninges (meningioma, meningiosarcoma, gliomatosis), cancers of the brain (astrocytoma, medulloblastoma, glioma, ependymoma, germinoma (pilealoma), glioblastoma multiform, oligodendroglioma, schwannoma, retinoblastoma, congenital tumors), and cancers of the spinal cord neurofibroma, meningioma, glioma, sarcoma); (4) hematologic cancers, including myeloid leukemia (acute and chronic), acute lymphloblastic leukemia, chronic lymphocytic leukemia, myeloproliferative diseases, multiple myeloma; myelodysplastic syndrome), Hodgkin's disease, and non-Hodgkin's lymphoma (malignant lymphoma); (5) cancers of the endocrine system, including: (a) cancers of the thyroid gland, including papillary carcinoma (including those with follicular foci), follicular carcinoma, medullary carcinoma, and undifferentiated (anaplastic) carcinoma; and (b) neuroblastomas, including sympathicoblastoma, sympathicogonioma, malignant ganglioneuroma, gangliosympathicoblastoma, and ganglioneuroma; (6) cancers of the skin, including squamous cell carcinoma, spindle cell variant of squamous cell carcinoma, basal cell carcinoma, adenocarcinoma developing from sweat or sebaceous gland, and malignant melanoma; (7) cancers of the eye, including: (a) cancers of the conjunctiva, including carcinoma of the conjunctiva; (b) cancers of the eyelid, including basal cell carcinoma, squamous cell carcinoma, melanoma of the eyelid, and sebaceous cell carcinoma; (c) cancers of the lacrimal gland, including adenocarcinoma, adenoid cystic carcinoma, carcinoma in pleomorphic adenoma, mucoepidermoid carcinoma, and squamous cell carcinoma; (d) cancers of the uvea, including spindle cell melanoma, mixed cell melanoma, and epithelioid cell melanoma; (e) cancers of the orbit, including sarcoma of the orbit, soft tissue tumor, and sarcoma of bone; and (f) retinoblastoma.

Examples of nontumor hyperproliferative disorders include but are not limited to myelodysplastic disorders; cervical carcinoma-in-situ; familial intestinal polyposes such as Gardner syndrome; oral leukoplakias; histiocytoses; keloids; hemangiomas; inflammatory arthritis; hyperkeratoses and papulosquamous eruptions including arthritis-related eruptions. Also included are viral induced hyperproliferative diseases such as warts and EBV induced disease (i.e., infectious mononucleosis), scar formation, blood vessel proliferative disorders such as restenosis, atherosclerosis, in-stent stenosis, vascular graft restenosis, etc.; fibrotic disorders; psoriasis; glomerular nephritis; macular degenerative disorders; benign growth disorders such as prostate enlargement and lipomas; autoimmune disorders and the like.

Compositions according to the present invention can also be administered for the treatment of cardiac dysrhythmias, including but not limited to the Wolff-Parkinson-White syndrome and atrioventricular nodal reentrant tachycardia ventricular tachycardia (VT), atrial tachycardias, atrial flutter and atrial fibrillation supraventricular tachycardias.

Compositions according to the present invention can also be administered for the treatment of endometriosis, uterine fibroid (uterine leiomyomata) menorrhagia, cervical erosion, cervical polyp, and related conditions.

Compositions according to the present invention can also be administered for the treatment of the defects or disorders of intervertebral discs including but not limited to annular fissures, fragmentation of the nucleus pulposus, contained herniation (a herniated intervertebral disc), and degenerative intervertebral discs.

Compositions according to the present invention can also be administered for the treatment of additional diseases or conditions, including, but not limited to, Alzheimer's disease, osteoporosis, sleep apnea, erectile dysfunction, McArdle disease, and carbohydrate metabolism disorders.

Compositions according to the present invention can also be administered for reducing aging or fatigue. As used herein, the term “reducing aging” refers to lessening, ameliorating, or relieving the deleterious effects of aging (e.g., low vigor, memory loss, weakened vision or hearing, and joint pain) in a subject. As used herein, the term “reducing fatigue” refers to lessening, ameliorating, or relieving one or more of the symptoms of fatigue (low energy, poor endurance, and attention deficits) in a subject.

The subject to be treated can be a human patient or a socially or economically important animal, including, but not limited to, a dog, a cat, a horse, a cow, a goat, a sheep, or a pig. Compositions according to the present invention can be formulated for treatment of non-human mammalian species such as, but not limited to, those described above and can be used in veterinary medicine. Methods according to the present invention are not limited to the treatment of humans and can be adapted for use in veterinary medicine.

The composition described above can be in dry form (e.g., powder or tablet) or in aqueous form (e.g., beverage or syrup). It can be a dietary supplement or a pharmaceutical formulation (containing a pharmaceutically acceptable carrier). It can also be a drink or a food product. Examples include tea (e.g., a tea drink and the contents of a tea bag), soft drinks, juice (e.g., a fruit extract and a juice drink), milk, coffee, cookies, cereals, chocolates, and snack bars.

The first and second agents described above include active compounds, as well as their salts, prodrugs, and solvates, if applicable. A salt, for example, can be formed between an anion and a positively charged group (e.g., amino) on an agent. Suitable anions include chloride, bromide, iodide, sulfate, nitrate, phosphate, citrate, methanesulfonate, trifluoroacetate, acetate, chlorophenyoxyacetate, malate, tosylate, tartrate, fumarate, glutamate, glucuronate, lactate, glutarate, benzoate, embonate, glycolate, pamoate, aspartate, parachlorophenoxyisobutyrate, formate, succinate, cyclohexanecarboxylate, hexanoate, octanoate, decanoate, hexadecanoate, octadecanoate, benzenesulphonate, trimethoxybenzoate, paratoluenesulphonate, adamantanecarboxylate, glycoxylate, pyrrolidonecarboxylate, naphthalenesulphonate, 1-glucosephosphate, sulfite, dithionate, and maleate. Likewise, a salt can also be formed between a cation and a negatively charged group (e.g., carboxylate) on an agent. Suitable cations include sodium ion, potassium ion, magnesium ion, calcium ion, and an ammonium cation such as tetramethylammonium ion. The agents also include salts containing quaternary nitrogen atoms. Examples of prodrugs include esters and other pharmaceutically acceptable derivatives, which, upon administration to a subject, are capable of providing active compounds. A solvate refers to a complex formed between an active compound and a pharmaceutically acceptable solvent. Examples of pharmaceutically acceptable solvents include water, ethanol, isopropanol, ethyl acetate, acetic acid, and ethanolamine.

In some alternatives, the composition can include one or more additional active ingredients unless such additional active ingredients are excluded by a definition of the composition that includes the phrase “consisting essentially of.”

The invention is illustrated by the following Example. This Example is included for illustrative purposes only, and is not intended to limit the invention.

### EXAMPLE

### Comparison of Weight Gain Inhibition Induced by Administration of Metformin Plus Melatonin with Weight Gain Inhibition Induced by Administration of Sibutramine to Rats

The purpose of this Example was to demonstrate and compare the abilities of metformin plus melatonin (AM) and sibutramine to influence weight gain reduction and other related obesity indicators in the Sprague-Dawley rat.

As detailed above, metformin is an AMPK activator, and melatonin is a serotonergic compound. Sibutramine is an oral anorexiant that is a centrally-acting serotonin-norepinephrine reuptake inhibitor structurally related to amphetamines, although its mechanism of action is distinct. It also significantly reduces the reuptake of dopamine. The action of sibutramine in enhancing satiety and therefore reducing appetite is thought to be related to its inhibition of reuptake of these neurotransmitters, especially serotonin.

Animals:

Male Sprague-Dawley rats were used with average body weight 223 g. The rats were fed with standard chow.

Groups and Treatment:

Before grouping, all animals were weighed at fasting state for 5 consecutive days. The rats were then divided into three groups (n=10) according to their body weight on Day 5 as shown in Table 1. The first dose of the drug as indicated in Table 1 was administered on the next day after grouping; the dose was administered by gavage (t.i.d.) in a solution dose of 1 mLkg body weight, and continued for a total of 65 days. Initially, each rat was treated accordingly with 0.25 mL drug solution; the dosage was then adjusted according to the body weight change of each rat, by increasing the dose by 0.05 mL for each 50 g body weight increment.

TABLE 1
Experimental Groups and Mixing Formulas

| Group | Treatment | Mixing Formula |
|---|---|---|
| G01 | Metformin + Melatonin (AM) | 8500 mg metformin and 60 |
| | | mg melatonin were |
| | | dissolved in GS that |
| | | contained 5% anhydrous |
| | | ethanol. The mixture was |
| | | then diluted to 100 mL. |
| G02 | Sibutramine (SIB) | 100 mg sibutramine was |
| | | dissolved in GS that |
| | | contained 5% anhydrous |
| | | ethanol. The mixture was |
| | | then diluted to 100 mL. |
| G03 | 10% Glucose Solution (GS) | 95 mL GS was mixed with |
| | | 5 mL anhydrous ethanol. |

Observations:

Groups were fed overnight with 30 g chowrat after drug administration and food deprived during daytime. Overnight food intake was measured every morning, while fasting body weight was measured every Tuesday and Friday night.

Statistical Analysis:

Statistical analysis was performed with SPSS software (IBM). One-way ANOVA was performed.

Results:

Weight Gain Inhibition:

As shown in Table 2 (g, n=10) after 65 days' treatment, the body weight of the AM and sibutramine groups had increased 257.6 g and 270.7 g, respectively, while the rats from the control group gained 292.7 g on average. The difference between the AM and the control groups was statistically more significant (p<0.01), compared to the difference between sibutramine and the control group (p<0.05). The weight loss ratios of AM and sibutramine were 6.8% and 4.3%, respectively.

TABLE 2
Weight Gain Inhibition Effect in Sprague-Dawley Rats

| | | | | | Relative | Weight |
|---|---|---|---|---|---|---|
| | | | | | Body | Loss |
| Group | Treatment | Pre-Dose | Post-Dose | ΔBW | Weight | Ratio |
| G01 | 85 mg/kg | 224.1 ± 9.1 | 481.7 ± 14.6** | 257.6 ± 11.8** | −35.1 | −6.8% |
| | Metformin + | | | | | |
| | 0.6 mg/kg | | | | | |
| | Melatonin | | | | | |
| | (AM) | | | | | |
| G02 | 1 mg/kg | 223.1 ± 8.4 | 493.8 ±13.0 | 270.7 ± 11.6* | −22.0 | −4.3% |
| | Sibutramine | | | | | |
| | (SIB) | | | | | |
| G03 | GS | 222.0 ± 8.2 | 514.7 ± 17.2 | 292.7 ± 15.7 | | |

(*p < 0.05,
**p < 0.01 vs. GS; all weights in grams)

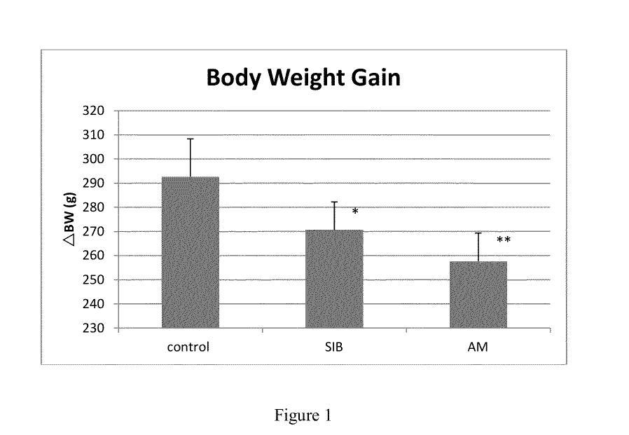

FIG. 1 shows the increase in body weight for the three groups.

Food Intake:

As shown in Table 3 (g, n=10), the daily food intakes of the AM and sibutramine groups were 29.5 g and 29.4 g, respectively, after 65 days of treatment. There were no significant differences for all the treatment groups in comparison to the control group (29.9 g).

TABLE 3
Food Intake

| | | Average Food | |
|---|---|---|---|
| | | Intake During | |
| Group | Treatment | Dosing, g | p-Value |
| G01 | AM | 29.5 ± 0.9 | 0.278 |
| G02 | SIB | 29.4 ± 0.9 | 0.240 |
| G03 | GS | 29.9 ± 0.2 | |

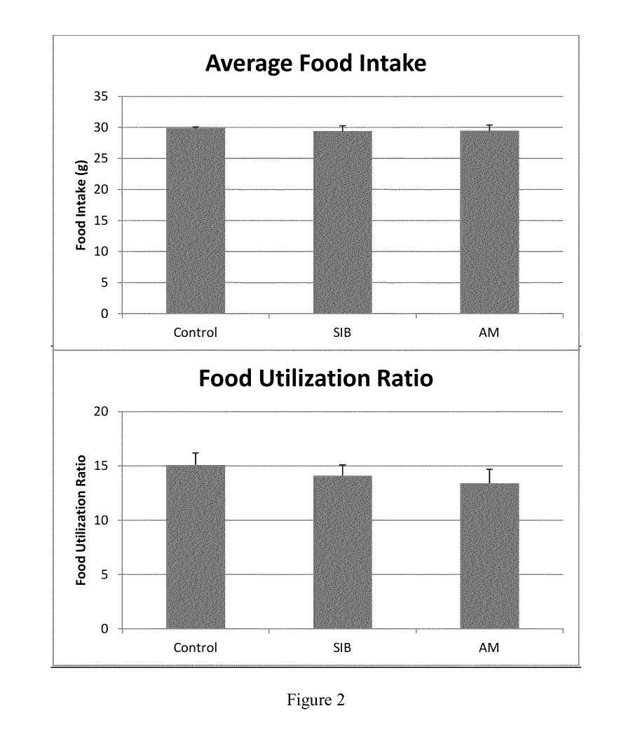

The average food intake for the groups is shown in FIG. 2.

Fat Mass:

As shown in Table 4 (g, n=10), the average fat mass of AM-treated and sibutramine-treated animals (19.9 g and 25.5 g, respectively), was less for each group than for the control group (26.1 g). Among all groups, only the difference between the control group and the AM-treated group was statistically significant (p<0.01).

TABLE 4
Fat Mass

| Group | Treatment | Fat Mass, g | p-Value |
|---|---|---|---|
| G01 | AM | 19.9 ± 6.2 | 0.009 |
| G02 | SIB | 25.5 ± 6.3 | 0.816 |
| G03 | GS | 26.1 ± 5.5 | |

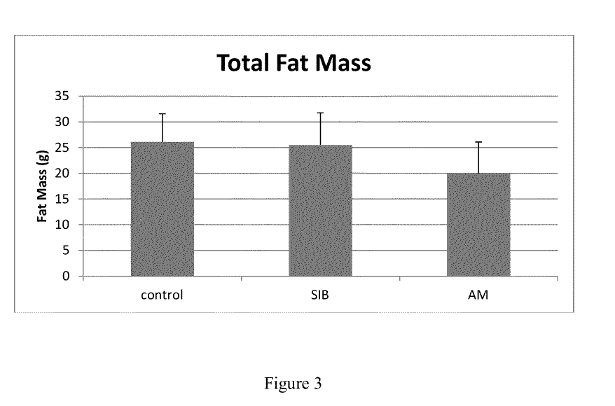

The results for total fat mass are shown in FIG. 3.

Conclusion:

After 65 consecutive days of treatment, AM and sibutramine not only decreased body weight gain, but also reduced fat mass of Sprague-Dawley rats, with AM (metformin plus melatonin) treatment demonstrating the greater degree of effectiveness, followed by sibutramine. During the dosing period, neither of the treatments affected the appetite of the rats.

### ADVANTAGES OF THE INVENTION

Compositions and methods according to the present invention are effective in treating a number of diseases and conditions, including metabolic syndrome and diseases and conditions associated with metabolic syndrome, hyperproliferative diseases including cancer, AIDS, Parkinson's disease, polycystic ovarian syndrome, Alzheimer's disease, osteoporosis, sleep apnea, erectile dysfunction, McArdle disease, and carbohydrate metabolism disorders, cardiac dysrhythmias; endometriosis, uterine fibroid (uterine leiomyomata) menorrhagia, cervical erosion, cervical polyp, and related conditions, defects or disorders of intervertebral discs. Compositions and methods according to the present invention are well tolerated, produce few if any side effects, and can be used together with other known pharmaceutically active compounds and compositions for treating these conditions.

Compositions and methods according to the present invention possess industrial applicability as compositions and methods for the preparation of a medicament to treat the diseases and conditions described above.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A pharmaceutical composition comprising:
(a) a therapeutically effective quantity of a first agent that is an activator of 5′-adenosine-monophosphate-activated kinase (AMPK); and
(b) a therapeutically effective quantity of a second agent that possesses or maintains serotonin activity.

2. The pharmaceutical composition of claim 1 wherein the AMPK activator is selected from the group consisting of: (1) metformin; (2) phenformin; (3) buformin; (4) AICAR; (5) a thienopyridone; (6) resveratrol; (7) nootkatone; (8) thiazole; (9) adiponectin; (10) 2-deoxyglucose; (11) AAPDs; (12) adiponectin variant polypeptides; (13) catechins; (14) trans-10, cis-12 conjugated linoleic acid; (15) a corydaline-related compound selected from the group consisting of corydaline, corlumidin, (+)-corlumidin, corypalmine, 14R-(+)-corypalmine, tetrahydropalmatine, 14R-(+)-tetrahydropalmatine, 14R,13S-(+)-corydaline, bicuculline, d-(+)-bicuculline, egenine, and +-egenine; (16) a dithiolethione; (17) an inhibitor or antagonist of DNA-dependent protein kinase catalytic subunit (DNA-PKcs); (18) a small interfering RNA (siRNA) that can inhibit the expression and/or translation of DNA-PKcs; (19) a fibrate selected from the group consisting of bezafibrate, ciprofibrate, fenofibrate, clofibrate, and gemfibrozil; (20) GW2974 (N4-(1-benzyl-1H-indazol-5-yl)-N6,N6-dimethyl-pyrido-[3,4-d]-pyrimidine-4,6-diamine); (21) honokiol; (22) leptin; (23) LKB1 (serinethreonine kinase 11); (24) obovatol (4′,5-diallyl-2,3-dihydroxybiphenyl ether); (25) a thiazolidinedione selected from the group consisting of pioglitazone and related thiazolidinediones, including rosiglitazone and rosiglitazone maleate; (26) a variant adiponectin peptide having one or more mutations at amino acid positions 109-229 of wild-type adiponectin and having at least threefold increased solubility when compared to wild-type adiponectin; (27) a butyrate compound selected from a butyrate salt and a butyrate ester; and (28) a quinoxalinedione derivative; and the salts, solvates, analogues, congeners, bioisosteres, hydrolysis products, metabolites, precursors, and prodrugs thereof.

3. The pharmaceutical composition of claim 2 wherein the AMPK activator is selected from the group consisting of metformin, phenformin, buformin, AICAR, thienopyridones, resveratrol, nootkatone, thiazole, adiponectin, thiazolidinediones, rosiglitazone, pioglitazone, dithiolethiones, and the salts, solvates, analogues, congeners, bioisosteres, hydrolysis products, metabolites, precursors, and prodrugs thereof.

4. The pharmaceutical composition of claim 3 wherein the AMPK activator is metformin or a salt thereof.

5. The pharmaceutical composition of claim 4 wherein the AMPK activator is metformin hydrochloride.

6. The pharmaceutical composition of claim 1 wherein the second agent is serotonin or a serotonin metabolite.

7. The pharmaceutical composition of claim 6 wherein the second agent is selected from the group consisting of serotonin sulfate, serotonin creatinine sulfate complex, serotonin hydrochloride, melatonin, 5-hydroyxindoleacetic acid, a salt of 5-hydroxyindoleacetic acid, melatonin creatinine sulfate complex, and 5-hydroxyindoleacetic acid creatinine sulfate complex.

8. The pharmaceutical composition of claim 7 wherein the second agent is melatonin or a salt thereof.

9. The pharmaceutical composition of claim 1 wherein the second agent is a serotonergic compound.

10. The pharmaceutical composition of claim 9 wherein the serotonergic compound is selected from the group consisting of:
(a) serotonin transport inhibitors;
(b) serotonin receptor 2C modulators;
(c) serotonin reuptake inhibitors;
(d) serotonin and norepinephrine reuptake inhibitors;
(e) serotonin dopamine antagonists;
(f) monoamine reuptake inhibitors;
(g) pyridazinone aldose reductase inhibitors;
(h) stimulants of serotonin receptors;
(i) stimulants of serotonin synthesis;
(j) serotonin agonists;
(k) serotonin receptor 1A antagonists; and
(l) serotonin metabolites.

11. The pharmaceutical composition of claim 10 wherein the second agent is selected from the group consisting of: (1) paroxetine; (2) fluoxetine; (3) fenfluramine; (4) fluvoxamine; (5) sertraline; (6) imipramine; (7) BVT933; (8) DPCA37215; (9) IK264; (10) PNU22394 (6-methyl-1,2,3,4,5,6-hexahydro-azepino[4,5-b]indole); (11) WAY161503 (8,9-dichloro-2,3,4,4a-tetrahydro-1H-pyrazino[1,2-a]quinoxalin-5(6H)-one hydrochloride); (12) R-1065; (13) YM348 ((2S)-1-(7-ethyl-1H-furo[2,3-g]indazol-1-yl)propan-2-amine); (14) milnacipran; (15) citalopram; (16) desmethylsertraline (a metabolite of sertraline); (17) norfluoxetine; (18) desmethylcitalopram (a metabolite of citalopram); (19) escitalopram; (20) femoxetine; (21) ifoxetine; (22) cyanodothiepin; (23) litoxetine; (24) dapoxetine; (25) nefazodone; (26) cericlamine; (27) trazodone; (28) mirtazapine; (29) indalpine; (30) indeloxazine; (31) sibutramine; (32) zimeldine; (33) (+)-N-[1-[1-(4-chlorophenyl)cyclobutyl]-3-methylbutyl}-N-methylamine; (34) (−)-N-{1-[1-(4-chlorophenyl)cyclobutyl-3-methylbutyl}-N-methylamine; (35) (−)-1-[1-(4-chlorophenyl)cyclobutyl]-3-methylbutylamine; (36) (+)-N-{1-[1-(4-chlorophenyl)cyclobutyl]-3-methylbutyl}-N; (37) (−)-N-{1-[1-(4-chlorophenyl)cyclobutyl]-3-methylbutyl}-N,N-dimethylamine)N-dimethylamine; (38) venlafaxine; (39) O-desmethylvenlafaxine (a metabolite of venlafaxine); (40) clomipramine; (41) desmethylclomipramine (a metabolite of clomipramine); (42) buspirone; (43) olanzapine; (44) ziprasidone; (45) ergoloid mesylates; (46) pergolide mesylate; (47) vitamin B1; (48) vitamin B3; (49) vitamin B6; (50) biotin; (51) S-adenosylmethionine; (52) folic acid; (53) folinic acid; (54) ascorbic acid; (55) magnesium; (56) coenzyme Q10; (57) piracetam; (58) (+)-2,5-dimethoxy-4-iodoamphetamine; (59) (+)-3,4-methylenedioxyamphetamine; (60) (+)-N-[2-[4-[2,3-dihydro-2-(hydroxymethyl)-1,4-benzodioxin-5-yl]1-piperazinyl]-4-fluorobenzamide hydrochloride; (61) (+)-norfenfluramine (a metabolite of fenfluramine); (62) (3β)-2,3-dihydrolysergene; (63) (3β)-2,3-dihydrolysergol; (64) (3β)-2,3-dihydro-methyllysergate; (65) (3β, 5β, 8β)-9,10-didehydro-2,3-dihydro-6-methyl-8-(2-pyridylthiomethyl) ergoline; (66) (3β, 5β, 8β)-9,10-didehydro-2,3-dihydro-6-methyl-8-(methylthiomethyl) ergoline; (67) (3β, 5β, 8β)-9,10-didehydro-2,3-dihydro-6-methyl-8-(phenylthiomethyl) ergoline; (68) (3β, 5β, 8β)-9,10-didehydro-2,3-dihydro-8-methyl-6-propylergoline; (69) 1-(4-bromo-2,5-dimethoxyphenyl)-2-aminopropane; (70) 1-(m-trifluoromethylphenyl)-piperazine; (71) 2-(4-(4-(2-pyrimidinyl)1-piperazinyl-propyl)-1,2-benzoisothiazol-3-(2H)-one 1,1-dioxide hydrochloride; (72) 2-methylserotonin; (73) 3β, 5β, 8β)-9,10-didehydro-2,3-dihydro-6-methylergoline-8-acetonitrile; (74) zolmitriptan; (75) 3a,4,4a,6a,7,7a-hexahydro-2-[4-[4-(2-pyrimidinyl)-11-piperazinyl]butyl]-4,7-etheno-1H-cyclobutanoisoindole-1,3(2H)-dione dihydrochloride sesquihydrate; (76) 3-butyl-9,9-dimethyl-7-[4-[4-[2-methoxyphenyl) 1-piperazinyl]butyl]-3,7-diazabicyclo[3,2,1]nonane-2,4,6,8-tetraone; (77) 4,4-dimethyl-1-[4-[4-(2-pyrimidinyl)-1-piperazinyl]butyl]2,6-piperidinedione hydrochloride; (78) 5-hydroxy-L-tryptophan; (79) 5-methoxy-N,N-dimethyltryptamine; (80) 6-[[3-[4[o-methoxyphenyl]-1-piperazinyl]propyl]-amino]-1,3-dimethyluracil; (81) 8-[4-N-[4-(2-pyrimidinyl)-1-piperazinyl]-butyl]-8-azaspiro[4.5]-decane-7,9-dione hydrochloride; (82) 8-hydroxy-2-(di-n-propylamino)tetralin (8-OH-DPAT); (83) alniditan; (84) almotriptan; (85) 2-aminotetralin; (86) bifeprunox; (87) gepirone; (88) BW723C86 (1-[5(2-thienylmethoxy)-1H-3-indolyl[propan-2-amine hydrochloride); (89) cisapride; (90) dihydroergotamine; (91) D-lysergic acid diethylamide; (92) donitriptan; (93) eletriptan; (94) frovatriptan; (95) tegaserod; (96) ipsapirone; (97) L694247 (2-[5-[3-(4-methylsulphonylamino)benzyl-1,2,4-oxadiazol-5-yl]-1H-indol-3yl]ethanamine); (98) cinitapride; (99) lesopitron; (100) MCPP (m-chlorophenylpiperazine); (101) methysergide; (102) metoclopramide; (103) MK-212 (6-chloro-2-(1-piperazinyl)pyrazine hydrochloride); (104) mosapride; (105) N,N-dimethyl-5-methoxytryptamine; (106) N,N-dimethyltryptamine; (107) N-[4-[4-(2-pyrimidinyl)-1-piperazinyl]butylbicyclo[2.2.1]heptane-2,3-di-oxo-carboximide; (108) naratriptan; (109) norcisapride; (110) phentermine; (111) quipazine; (112) prucalopride; (113) rauwolscine; (114) repinotan; (115) rizatriptan; (116) sumatriptan; (117) tandospirone; (118) 1-methyl-4-phenyl-1,2,3,6-tetrahydropyridine; (119) tiaspirone; (120) trifluoromethylphenylpiperazine; (121) L-tryptophan; (122) xaliproden; (123) yohimbine; (124) zacopride; (125) zalospirone (126) mianserin; (127) setiptiline; (128) adatanserin; (129) altanserin; (130) benanserin; (131) blonanserin; (132) butanserin; (133) cinanserin; (134) eplivanserin; (135) flibanserin (136) glemanserin; (137) iferanserin; (138) ketanserin; (139) lidanserin; (140) pelanserin; (141) pruvanserin; (142) ritanserin; (143) seganserin; (144) tropanserin; (145) iloperidone; (146) sertindole; (147) EMR-62218; (148) asenapine; (149) zotepine; (150) ocaperidone; (151) APD125; (152) AVE8488; (153) pimavanserin; (154) isocarboxazid; (155) phenelzine; (156) tranylcypromine; (157) amitriptyline; (158) clomipramine; (159) N-(1-(1-methylethyl)piperidin-4-yl)-N-((4-methylphenyl)methyl)-4-methoxyphenylacetamide; (160) N-(1-(2,2-dimethylethyl)piperidin-4-yl)-N-((4-methylphenyl)methyl)-4-methoxyphenylacetamide; (161) N-(1-pentylpiperidin-4-yl)-N-((4-methylphenyl)methyl)-4-methoxyphenylacetamide; (162) N-(1-hexylpiperidin-4-yl)-N-((4-methylphenyl)methyl)-4-methoxyphenylacetamide; (163) N-(1-cyclohexylpiperidin-4-yl)-N-((4-methylphenyl)methyl)-4-methoxyphenylacetamide; (164) N-(1-cyclopentylpiperidin-4-yl)-N-((4-methylphenyl)methyl)-4-methoxyphenylacetamide; (165) N-(1-cyclobutylpiperidin-4-yl)-N-((4-methylphenyl)methyl)-4-methoxyphenylacetamide; (166) N-(1-cyclopropylpiperidin-4-yl)-N-((4-methylphenyl)methyl)-4-methoxyphenylacetamide; (167) N-(1-(cyclopentylmethyl)piperidin-4-yl)-N-((4-methylphenyl)methyl)-4-methoxyphenylacetamide; (168) N-(1-(cyclobutylmethyl)piperidin-4-yl)-N-((4-methylphenyl)methyl)-4-methoxyphenylacetamide; (169) N-(1-(cyclopropylmethyl)piperidin-4-yl)-N-((4-methylphenyl)methyl)-4-methoxyphenylacetamide; (170) N-(1-(2-hydroxyethyl)piperidin-4-yl)-N-((4-methylphenyl)methyl)-4-methoxyphenylacetamide; (171) N-(1-(3-hydroxypropyl)piperidin-4-yl)-N-((4-methylphenyl)methyl)-4-methoxyphenylacetamide; (172) N-((4-Methylphenyl)methyl)-N-(piperidin-4-yl)-N′-phenylmethylcarbamide; (173) N-((4-Methylphenyl)methyl)-N-(1-(2-methylpropyl)piperidin-4-yl)-N′-phenylmethylcarbamide; (174) N-(1-((2-Bromophenyl)methyl)piperidin-4-yl)-N-((4-methylphenyl)methyl)-W-phenylmethylcarbamide; (175) N-(1-((4-Hydroxy-3-methoxyphenyl)methyl)piperidin-4-yl)-N-((4-methylphenyl) methyl)-N′-phenylmethylcarbamide; (176) N-(1-((5-Ethylthien-2-yl)methyl)piperidin-4-yl)-N-((4-methylphenyl)methyl)-N′-phenylmethylcarbamide; (177) N-(1-(Imidazol-2-ylmethyl)piperidin-4-yl)-N-((4-methylphenyl)methyl)-N′-phenylmethylcarbamide; (178) N-(1-(Cyclohexylmethyl)piperidin-4-yl)-N-((4-methylphenyl)methyl)-N′-phenylmethylcarbamide; (179) N-(1-((4-Fluorophenyl)methyl)pipericlin-4-yl)-N-((4-methylphenyl)methyl)-N′-phenylmethylcarbamide; (180) N-((4-Methylphenyl)methyl)-N-(piperidin-4-yl)-4-methoxyphenylacetamide; (181) N-((4-Methylphenyl)methyl)-N-(1-methylpiperidin-4-yl)-4-methoxyphenylacetamide; (182) N-(1-Ethylpiperidin-4-yl)-N-((4-methylphenyl)methyl)-4-methoxyphenylacetamide; (183) N-((4-Methylphenyl)methyl)-N-(1-propylpiperidin-4-yl)-4-methoxyphenylacetamide; (184) N-(1-Butylpiperidin-4-yl)-N-((4-methylphenyl)methyl)-4-methoxyphenylacetamide; (185) N-(1-(3,3-Dimethylbutyl)piperidin-4-yl)-N-((4-methylphenyl)methyl)-4-methoxyphenylacetamide; (186) N-(1-(Cyclohexylmethyl)piperidin-4-yl)-N-((4-methylphenyl)methyl)-4-methoxyphenylacetamide; (187) N-((4-Methylphenyl)methyl)-N-(1-(2-methylpropyl)piperidin-4-yl)-4-methoxyphenylacetamide; (188) N-((4-Methylphenyl)methyl)-N-(1-((4-methylphenyl)methyl)piperidin-4-yl)-4-methoxyphenylacetamide; (189) N-(1-((4-Hydroxyphenyl)methyl)piperidin-4-yl)-N-((4-methylphenyl)methyl)-4-methoxyphenylacetamide; (190) N-(1-((2-Hydroxyphenyl)methyl)piperidin-4-yl)-N-((4-methylphenyl)methyl)-4-methoxyphenylacetamide; (191) N-(3-Phenylpropyl)-N-(piperidin-4-yl)-4-methoxyphenylacetamide; (192) N-(2-Phenylethyl)-N-(piperidin-4-yl)-4-methoxyphenylacetamide; (193) N-((2-Methoxyphenyl)methyl)-N-(piperidin-4-yl)-4-methoxyphenylacetamide; (194) N-((2-Chlorophenyl)methyl)-N-(piperidin-4-yl)-4-methoxyphenylacetamide; (195) N-((3,4-Di-methoxyphenyl)methyl)-N-(pipericlin-4-yl)-4-methoxyphenylacetamide; (196) N-((4-Fluorophenyl)methyl)-N-(piperidin-4-yl)-4-methoxyphenylacetamide; (197) N-((2,4-Di-chlorophenyl)methyl)-N-(piperidin-4-yl)-4-methoxyphenylacetamide; (198) N-((3-Methylphenyl)methyl)-N-(pipericlin-4-yl)-4-methoxyphenylacetamide; (199) N-((3-Bromophenyl)methyl)-N-(pipericlin-4-yl)-4-methoxyphenylacetamide; (200) N-(1-(Phenylmethyl)piperidin-4-yl)-N-(3-phenyl-2-propen-1-yl)-4-methoxyphenylacetamide; (201) N-((4-Methylphenyl)methyl)-N-(1-pipericlin-4-yl)-phenylacetamide; (202) N-((4-Methylphenyl)methyl)-N-(1-piperidin-4-yl)-3-phenylpropionamide; (203) N-((4-Methylphenyl)methyl)-N-(1-piperidin-4-yl)-(phenylthio)acetamide; (204) N-((4-Methylphenyl)methyl)-N-(1-piperidin-4-yl)-phenoxyacetamide; (205) N-((4-Methylphenyl)methyl)-N-(1-piperidin-4-yl)-(4-chlorophenoxy)acetamide; (206) N-((4-Methylphenyl)methyl)-N-(1-piperidin-4-yl)-3-methoxyphenylacetamide; (207) N-((4-Methylphenyl)methyl)-N-(1-piperidin-4-yl)-4-fluorophenylacetamide; (208) N-((4-Methylphenyl)methyl)-N-(1-piperidin-4-yl)-2,5-di-methoxyphenylacetamide; (209) N-((4-Methylphenyl)methyl)-N-(1-piperidin-4-yl)-4-chlorophenylacetamide; (210) N-((4-Methylphenyl)methyl)-N-(1-(phenylmethyl)pyrrolidin-3-yl)-N′-phenylmethylcarbamide; (211) N-((4-Methylphenyl)methyl)-N-(1-(phenylmethyl)pyrrolidin-3-yl)-4-methoxyphenylacetamide; (212) 2-(4-methoxyphenyl)-N-(4-methylbenzyl)-N-(piperidin-4-yl)acetamide; (213) 2-(4-methoxyphenyl)-N-(4-methylbenzyl)-N-(1-methylpiperidin-4-yl)acetamide; (214) 2-(4-methoxyphenyl)-N-(4-methylbenzyl)-N-(1-ethylpiperidin-4-yl)acetamide; (215) 2-(4-methoxyphenyl)-N-(4-chlorbenzyl)-N-(1-ethylpiperidin-4-yl)acetamide; (216) 2-(4-methoxyphenyl)-N-(4-chlorbenzyl)-N-(1-isopropylpiperidin-4-yl)acetamide; (217) 2-(4-methoxyphenyl)-N-(4-chlorobenzyl)-N-(piperidin-4-yl)acetamide; (218) 2-(4-methoxyphenyl)-N-(4-chlorobenzyl)-N-(1-cyclopentylpiperidin-4-yl)acetamide; (219) 2-(4-methoxyphenyl)-N-(4-chlorbenzyl)-N-(1-isopropylpiperidin-4-yl)acetamide; (220) 2-(phenyl)-N-(4-trifluoromethylbenzyl)-N-(1-methylpiperidin-4-yl)acetamide; (221) 2-(4-fluorophenyl)-N-(4-trifluoromethylbenzyl)-N-(1-methylpiperidin-4-yl)acetamide; (222) 2-(4-Methoxyphenyl)-N-(4-trifluoromethylbenzyl)-N-(1-methylpiperidin-4-yl)acetamide; (223) 2-(4-Trifluoromethylphenyl)-N-(4-trifluoromethylbenzyl)-N-(1-methylpiperidin-4-yl)acetamide; (224) 2-(4-Fluorophenyl)-N-(4-fluorobenzyl)-N-(1-methylpiperidin-4-yl)acetamide; (225) 2-(4-Methoxyphenyl)-N-(4-fluorobenzyl)-N-(1-methylpiperidin-4-yl)acetamide; (226) 2-(phenyl)-N-(4-fluorobenzyl)-N-(1-methylpiperidin-4-yl)acetamide; (227) 2-(4-Trifluoromethylphenyl)-N-(4-fluorobenzyl)-N-(1-methylpiperidin-4-yl)acetamide; (228) 2-(4-trifluoromethylphenyl)-N-[4-(methoxycarbonyl)benzyl]-N-(1-methylpiperidin-4-yl)acetamide; (229) 2-Phenyl-N-[4-(methoxycarbonyl)benzyl]-N-(1-methylpiperidin-4-yl)acetamide; (230) 2-(4-Chlorophenyl)-N-[4-(methoxycarbonyl)benzyl]-N-(1-methylpiperidin-4-yl) acetamide; (231) 2-(4-Methoxyphenyl)-N-[4-(methoxycarbonyl)benzyl]-N-(1-methylpiperidin-4-yl)acetamide; (232) 2-(4-trifluoromethylphenyl)-N-[4-(methoxycarbonyl)benzyl]-N-(1-methylpiperidin-4-yl)acetamide; (233) 2-Phenyl-N-[4-(methoxycarbonyl)benzyl]-N-(1-methylpiperidin-4-yl)acetamide; (234) 2-(4-Chlorophenyl)-N-[4-(methoxycarbonyl)benzyl]-N-(1-methylpiperidin-4-yl) acetamide; (235) 2-(4-Methoxyphenyl)-N-[4-(methoxycarbonyl)benzyl]-N-(1-methylpiperidin-4-yl)acetamide; (236) 2-(4-methoxyphenyl)-N-(4-methylbenzyl)-N-[1-(4-chloromethyl-2-thiazolylmethyl)piperidin-4-yl]acetamide; (237) 2-(4 methoxyphenyl)-N-(4-methylbenzyl)-N-{1-[3-(1,3-dihydro-2H-benzimidazol-2-on-1-yl)propyl]piperidin-4-yl}acetamide; (238) 2-(4-methoxyphenyl)-N-(2-4(fluorophenyl)ethyl)-N-(1-methylpiperidin-4-yl)acetamide; (239) 2-(4-methoxyphenyl)-N-[2-(2,5-dimethoxyphenyl)ethyl]-N-(1-methylpiperidin-4-yl)acetamide; (240) 2-(4-methoxyphenyl)-N-[2-(2,4-dichlorophenyl)ethyl]-N-(1-methylpiperidin-4-yl)acetamide; (241) 2-(4-methoxyphenyl)-N-[2-(3-chlorophenyl)ethyl]-N-(1-methylpiperidin-4-yl)acetamide; (242) 2-(4-methoxyphenyl)-N-[2-(4-methoxyphenyl)ethyl]-N-(1-methylpiperidin-4-yl) acetamide; (243) 2-(4-methoxyphenyl)-N-[2-(3-fluorophenyl)ethyl]-N-(1-methylpiperidin-4-yl)acetamide; (244) 2-(4-ethoxyphenyl)-N-[2-(4-fluorophenethyl]-N-(1-methylpiperidin-4-yl)acetamide; (245) 2-(4-ethoxyphenyl)-N-(4-fluorobenzyl)-N-(1-methylpiperidin-4-yl)acetamide; (246) 2-(4-methoxyphenyl)-N-(4-methylbenzyl)-N-{1-[2-(2-hydroxyethoxy)ethyl]piperidin-4-yl}acetamide; (247) 2-(4-methoxyphenyl)-N-(4-methylbenzyl)-N-[1-((2-chloro-5-thienyl)methyl) piperidin-4-yl]acetamide; (248) 2-(4-methoxyphenyl)-N-(4-methylbenzyl)-N-[1-(2-(imidazolidinon-1-yl)ethyl)piperidin-4-yl]acetamide; (249) 2-(4-methoxyphenyl)-N-(4-methylbenzyl)-N-{1-[2-(2,4(1H,3H)quinazolinedion-3-yl)ethyl]piperidin-4-yl}acetamide; (250) 2-(4-methoxyphenyl)-N-(4-methylbenzyl)-N-{1-[2-(1,3-dioxolan-2-yl)ethyl]piperidin-4-yl}acetamide; (251) 2-(4-methoxyphenyl)-N-(4-methylbenzyl)-N-{1-[2-(3-indolyl)ethyl]piperidin-4-yl}acetamide; (252) 2-(4-methoxyphenyl)-N-(4-methylbenzyl)-N-{1-[3-(1,2,4-triazol-1-yl)propyl]piperidin-4-yl}acetamide; (253) 2-(4-methoxyphenyl)-N-(4-methylbenzyl)-N-[1-(5-benzofurazanylmethyl)piperidin-4-yl]acetamide; (254) 2-(4-methoxyphenyl)-N-(4-methylbenzyl)-N-[1-(5-chlorobenzo[b]thien-3-ylmethyl)piperidin-4-yl]acetamide; (255) 2-(4-methoxyphenyl)-N-(4-methylbenzyl)-N-[1-(5-phenyl-1,2,4-oxadiazol-3-ylmethyl)piperidin-4-yl]acetamide; (256) 2-(4-Chlorophenyl)-N-(4-methylbenzyl)-N-(1-isopropylpiperidin-4-yl)-acetamide; (257) 2-(4-Chlorophenyl)-N-(4-methylbenzyl)-N-(1-ethylpiperidin-4-yl)-acetamide; (258) 2-Phenyl-N-(4-methylbenzyl)-N-(1-methylpiperidin-4-yl)-acetamide; (259) 2-(4-Chlorophenyl)-N-(4-methylbenzyl)-N-(1-methylpiperidin-4-yl)-acetamide; (260) 2-(4-Chlorophenyl)-N-(4-methylbenzyl)-N-(1-cyclopentylpiperidin-4-yl)-acetamide; (261) 2-(4-Fluorophenyl)-N-(4-methylbenzyl)-N-(1-methylpiperidin-4-yl)-acetamide; (262) 2-(4-Chlorophenyl)-N-(4-methylbenzyl)-N-(1-(2-hydroxyethyl)-piperidin-4-yl)-acetamide; (263) 2-(4-Chlorophenyl)-N-(4-methylbenzyl)-N-(1-cyclobutylpiperidin-4-yl)-acetamide; (264) 2-(4-Methoxyphenyl)-N-(4-methylbenzyl)-N-(1-cyclobutylpiperidin-4-yl)-acetamide; (265) 2-(4-Methoxyphenyl)-N-(4-methylbenzyl)-N-(tropin-4-yl)-acetamide; (266) N-(4-Methylbenzyl)-N-(1-methylpiperidin-4-yl)-N′-benzyl-carbamide; (267) N-(4-Methylbenzyl)-N-(1-methylpiperidin-4-yl)-N′-phenyl-carbamide; (268) N-Phenethyl-N-(1-methylpiperidin-4-yl)-N′-benzyl-carbamide; (269) 2-Phenyl-N-(4-methoxybenzyl)-N-(1-methylpiperidin-4-yl)-acetamide; (270) 2-(4-Trifluoromethylphenyl)-N-(4-methoxybenzyl)-N-(1-methylpiperidin-4-yl)-acetamide (271) 2-(4-Fluorophenyl)-N-(4-methoxybenzyl)-N-(1-methylpiperidin-4-yl)-acetamide; (272) 2-(4-Methoxyphenyl)-N-(4-methoxybenzyl)-N-(1-methylpiperidin-4-yl)-acetamide; (273) 2-(4-Methylphenyl)-N-(4-chlorobenzyl)-N-(1-methylpiperidin-4-yl)-acetamide; (274) 2-(4-Hydroxyphenyl)-N-(4-methylbenzyl)-N-(1-methylpiperidin-4-yl)-acetamide; (275) N-Phenethyl-N-(1-methylpiperidin-4-yl)-N′-phenyl-carbamide; (276) N-(3-Phenylpropyl)-N-(1-methylpiperidin-4-yl)-N′-benzyl-carbamide; (277) N-(3-Phenylpropyl)-N-(1-methylpiperidin-4-yl)-N′-phenyl-carbamide; (278) 2-(4-Methoxyphenyl)-2,2-ethylene-N-(4-methylbenzyl)-N-(1-methylpiperidin-4-yl)acetamide; (279) 2-(4-Methoxyphenyl)-N-alpha-methylbenzyl-N-(1-methylpiperidin-4-yl)acetamide; (280) 2-(4-Methoxyphenyl)-N-(4-methylbenzyl)-N-(3-tropen-4-ypacetamide; (281) 2-Phenyl-2-ethyl-N-(4-methylbenzyl)-N-(1-methylpiperidin-4-yl)acetamide; (282) N-Phenethyl-N-(4-methylbenzyl)-N-(1-methylpiperidin-4-yl)-amine; (283) 2-(4-Methoxyphenyl)-N-(1-indanyl)-N-(1-methylpiperidin-4-yl)acetamide; (284) N-(4-Methylbenzyl)-N-(1-methylpiperidin-4-yl)-N′-(4-methoxybenzyl)-carbamide; (285) 2-(3,4-dimethoxyphenyl)-N-(4-methylbenzyl)-N-(1-methylpiperidin-4-yl)acetamide; (286) 2-(3,4-Methylenedioxyphenyl)-N-(4-methylbenzyl)-N-(1-methylpiperidin-4-yl)acetamide; (287) 2-(4-Methoxyphenyl)-N-(4-methylbenzyl)-N-(1-t-butylpiperidin-4-yl)-acetamide; (288) N-(4-Methylbenzyl)-N-(1-methylpiperidin-4-yl)-N′-phenethyl-carbamide; (289) N-Phenethyl-N-(1-methylpiperidin-4-yl)-N′-phenethyl-carbamide; (290) N-(4-Methylbenzyl)-N-(1-t-butylpiperidin-4-yl)-N′-(4-methoxybenzyl)-carbamide; (291) 2-(4-Ethoxyphenyl)-N-(4-methylbenzyl)-N-(1-methylpiperidin-4-yl)acetamide; (292) 2-(4-Butoxyphenyl)-N-(4-methylbenzyl)-N-(1-methylpiperidin-4-yl)acetamide; (293) 2-(4-i-Propoxyphenyl)-N-(4-methylbenzyl)-N-(1-methylpiperidin-4-yl)acetamide; (294) 2-(4-t-Butoxyphenyl)-N-(4-methylbenzyl)-N-(1-methylpiperidin-4-yl)acetamide; (295) 2-(4-Butoxyphenyl)-N-(4-fluorobenzyl)-N-(1-methylpiperidin-4-yl)acetamide; (296) 2-(4-Propoxyphenyl)-N-(4-fluorobenzyl)-N-(1-methylpiperidin-4-yl)acetamide; (297) 2-(4-i-Propoxyphenyl)-N-(4-fluorobenzyl)-N-(1-methylpiperidin-4-yl)acetamide; (298) 2-(4-t-Butoxyphenyl)-N-(4-fluorobenzyl)-N-(1-methylpiperidin-4-yl)acetamide; (299) 4-(4-Fluorobenzyl)-3-(4-methoxybenzyl)-8-methyl-1-oxa-3,8-diaza-spiro[4.5]decan-2-one; (300) 3-(4-Ethoxybenzyl)-4-(4-fluorobenzyl)-8-methyl-1-oxa-3,8-diaza-spiro[4.5]decan-2-one; (301) 4-(4-Fluorobenzyl)-8-methyl-3-(4-propoxybenzyl)-1-oxa-3,8-diaza-spiro[4.5]decan-2-one; (302) 3-(4-Cyclopropylmethoxybenzyl)-4-(4-fluorobenzyl)-8-methyl-1-oxa-3,8-diaza-spiro[4.5]decan-2-one; (303) 4-(4-Fluorobenzyl)-3-(4-isopropoxybenzyl)-8-methyl-1-oxa-3,8-diaza-spiro[4.5]decan-2-one; (304) 3-(4-Butoxybenzyl)-4-(4-fluorobenzyl)-8-methyl-1-oxa-3,8-diaza-spiro[4.5]decan-2-one; (305) 4-(4-Fluorobenzyl)-3-(4-isobutoxybenzyl)-8-methyl-1-oxa-3,8-diaza-spiro[4.5]decan-2-one; (306) 3-(4-Difluoromethoxybenzyl)-4-(4-fluorobenzyl)-8-methyl-1-oxa-3,8-diaza-spiro[4.5]decan-2-one; (307) 4-(4-Fluorobenzyl)-8-methyl-3-(4-trifluoromethoxybenzyl)-1-oxa-3,8-diaza-spiro[4.5]decan-2-one; (308) 4-(4-Fluorobenzyl)-8-methyl-3-(4-pentoxybenzyl)-1-oxa-3,8-diaza-spiro[4.5]decan-2-one; (309) 8-Ethyl-4-(4-fluorobenzyl)-3-(4-isobutoxybenzyl)-1-oxa-3,8-diaza-spiro[4.5]decan-2-one; (310) 4-(4-Fluorobenzyl)-3-(4-isobutoxybenzyl)-8-isopropyl-1-oxa-3,8-diaza-spiro[4.5]decan-2-one; (311) 8-Cyclopropylmethyl-4-(4-fluorobenzyl)-3-(4-isobutoxybenzyl)-1-oxa-3,8-diaza-spiro[4.5]decan-2-one; (312) 8-Cyclohexylmethyl-4-(4-fluorobenzyl)-3-(4-isobutoxybenzyl)-1-oxa-3,8-d iaza-spiro[4.5]decan-2-one; (313) 8-Cyclopentyl-4-(4-fluorobenzyl)-3-(4-isobutoxybenzyl)-1-oxa-3,8-diaza-spiro[4.5]decan-2-one; (314) 4-(4-Fluorobenzyl)-3-(4-isobutoxybenzyl)-8-(3-morpholin-4-yl-propyl)-1-oxa-3,8-diaza-spiro[4.5]decan-2-one; (315) 8-(2-[1,3]Dioxolan-2-yl-ethyl)-4-(4-fluorobenzyl)-3-(4-isobutoxybenzyl)-1-oxa-3,8-diaza-spiro[4.5]decan-2-one; (316) 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one; (317) 4-(4-Fluorobenzyl)-3-(4-isobutoxybenzyl)-8-[3-(2-oxo-2,3-dihydro-benzoimid azol-1-yl)-propyl]-1-oxa-3,8-diaza-spiro[4.5]decan-2-one; (318) 4-(4-Fluorobenzyl)-3-(4-isobutoxybenzyl)-8-(2-methyl-thiazol-4-yl-methyl)-1-oxa-3,8-diaza-spiro[4.5]decan-2-one; (319) 4-(4-Chlorobenzyl)-3-(4-isobutoxybenzyl)-8-methyl-1-oxa-3,8-diaza-spiro[4.5]decan-2-one; (320) 8-Ethyl-4-(4-chlorobenzyl)-3-(4-isobutoxybenzyl)-1-oxa-3,8-diaza-spiro[4.5]decan-2-one; (321) 4-(4-Chlorobenzyl)-3-(4-isobutoxybenzyl)-8-isopropyl-1-oxa-3,8-diaza-spiro[4.5]decan-2-one; (322) 8-Cyclopropylmethyl-4-(4-chlorobenzyl)-3-(4-isobutoxybenzyl)-1-oxa-3,8-diaza-spiro[4.5]decan-2-one; (323) 8-Cyclohexylmethyl-4-(4-chlorobenzyl)-3-(4-isobutoxybenzyl)-1-oxa-3,8-diaza-spiro[4.5]decan-2-one; (324) 8-(2-[1,3]Dioxolan-2-yl-ethyl)-4-(4-chlorobenzyl)-3-(4-isobutoxybenzyl)-1-oxa-3,8-diaza-spiro[4.5]decan-2-one; (325) 4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one; (326) 3-(4-Difluoromethoxybenzyl)-4-(4-fluorobenzyl)-8-methyl-1-oxa-3,8-diaza-spiro[4.5]decan-2-one; (327) 3-(4-Difluoromethoxybenzyl)-8-ethyl-4-(4-fluorobenzyl)-1-oxa-3,8-diaza-spiro[4.5]decan-2-one; (328) 3-(4-Difluoromethoxybenzyl)-4-(4-fluorobenzyl)-8-isopropyl-1-oxa-3,8-diaza-spiro[4.5]decan-2-one; (329) 8-Cyclopropylmethyl-3-(4-difluoromethoxybenzyl)-4-(4-fluorobenzyl)-1-oxa-3,8-diaza-spiro[4.5]decan-2-one; (330) 8-Cyclohexylmethyl-3-(4-difluoromethoxybenzyl)-4-(4-fluorobenzyl)-1-oxa-3,8-diaza-spiro[4.5]decan-2-one; (331) 3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one; (332) 3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one; (333) 8-Ethyl-4-(4-fluorobenzyl)-3-(4-trifluoromethoxybenzyl)-1-oxa-3,8-diaza-spiro[4.5]decan-2-one; (334) 4-(4-Fluorobenzyl)-8-isopropyl-3-(4-trifluoromethoxybenzyl)-1-oxa-3,8-diaza-spiro[4.5]decan-2-one; (335) 8-Cyclopropylmethyl-4-(4-fluorobenzyl)-3-(4-trifluoromethoxybenzyl)-1-oxa-3,8-diaza-spiro[4.5]decan-2-one; (336) 8-Cyclohexylmethyl-4-(4-fluorobenzyl)-3-(4-trifluoromethoxybenzyl)-1-oxa-3,8-diaza-spiro[4.5]decan-2-one; (337) 8-Cyclopentyl-4-(4-fluorobenzyl)-3-(4-trifluoromethoxybenzyl)-1-oxa-3,8-diaza-spiro[4.5]decan-2-one; (338) 8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one; (339) 4-(4-Fluorobenzyl)-8-[2-(2-oxo-imidazolidin-1-yl)-ethyl]-3-(4-trifluoromethoxybenzyl)-1-oxa-3,8-diaza-spiro[4.5]decan-2-one; (340) 8-Ethyl-4-(4-fluorobenzyl)-3-(4-propoxybenzyl)-1-oxa-3,8-diaza-spiro[4.5]decan-2-one; (341) 4-(4-Fluorobenzyl)-8-isopropyl-3-(4-propoxybenzyl)-1-oxa-3,8-diaza-spiro[4.5]decan-2-one; (342) 8-Cyclopropylmethyl-4-(4-fluorobenzyl)-3-(4-propoxybenzyl)-1-oxa-3,8-diaza-spiro[4.5]decan-2-one; (343) 8-Cyclohexylmethyl-4-(4-fluorobenzyl)-3-(4-propoxybenzyl)-1-oxa-3,8-diaza-spiro[4.5]decan-2-one; (344) 8-Cyclopentyl-4-(4-fluorobenzyl)-3-(4-propoxybenzyl)-1-oxa-3,8-diaza-spiro[4.5]decan-2-one; (345) 8-(2-[1,3]Dioxolan-2-yl-ethyl)-4-(4-fluorobenzyl)-3-(4-propoxybenzyl)-1-oxa-3,8-diaza-spiro[4.5]decan-2-one; (346) 4-(4-Fluorobenzyl)-8-[2-(2-oxo-imidazolidin-1-yl)-ethyl]-3-(4-propoxybenzyl)-1-oxa-3,8-diaza-spiro[4.5]decan-2-one; (347) 3-(4-Cyclopropylmethoxybenzyl)-8-ethyl-4-(4-fluorobenzyl)-1-oxa-3,8-diaza-spiro[4.5]decan-2-one; (348) 3-(4-Cyclopropylmethoxybenzyl)-4-(4-fluorobenzyl)-8-isopropyl-1-oxa-3,8-diaza-spiro[4.5]decan-2-one; (349) 3-(4-Cyclopropylmethoxybenzyl)-8-cyclopropylmethyl-4-(4-fluorobenzyl)-1-oxa-3,8-diaza-spiro[4.5]decan-2-one; (350) 3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one; (351) 3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one; (352) 8-(2-[1.3]-Dioxan-2-yl-ethyl)-4-(4-fluorobenzyl)-3-(4-isobutoxybenzyl)-1-oxa-3,8-diaza-spiro[4.5]decane-3-one; (353) 4-(4-Fluorobenzyl)-3-(4-isobutoxybenzyl)-8-{3-[(S)-4-isopropyl-2-oxo-oxazolidin-3-yl]-propyl}-1-oxa-3,8-diaza-spiro[4.5]decane-3-one; (354) N-{1-[2-(1,3-Dioxolan-2-yl)ethyl]piperidin-4-yl}-N-(4-fluorobenzyl)-N′-(4-isobutoxybenzyl)carbamide hydrochloride; (355) N-{1-[2-(1,3-Dioxan-2-yl)ethyl]-piperidin-4-yl}-N-(4-fluorobenzyl)-2-[4-(2-hydroxy-2-methylpropoxy)phenyl]-acetamide tartrate; (356) N-(4-Fluorobenyzl)-N-(piperidin-4-yl)-2-(4-isobutoxyphenyl)acetamide; (357) N-{1-[3-(3,5-Dimethylpiperidin-1-yl)propyl]piperidin-4-yl-}-(4-fluorobenzyl)-2-(4-isobutoxyphenyl)acetamide dihydrochloride; (358) 1-[3-(4-{(4-Fluorobenzyl)-[2-(4isobutoxyphenyl)acetyl]amino}piperidin-1-yl)propyl]piperidine-4-carboxylic acid methyl ester dihydrochloride; (359) N-(4-Fluorobenzyl)-2-(4-isobutoxyphenyl)-N-{1-[2-(1-methylpyrrolidin-2-yl-)ethyl]piperidin-4-yl}acetamide dioxalate; (360) N-{1-[3-(2,6-Dimethylmorpholin-4-yl)propyl]piperidin-4-yl}-N-(4-fluorobenzyl)-2-(4-isobutoxyphenyl)acetamide dioxalate; (361) N-(4-Fluorobenzyl)-N-{1-[3-(3-hydroxypiperidin-1-yl)propyl]piperidin-4-yl}-2-(4-isobutoxyphenyl)acetamide dioxalate; (362) N-(4-Fluorobenzyl)-2-(4-isobutoxyphenyl)-N-{1-[3-(2-methylpiperidin-1-yl)-propyl]piperidin-4-yl}acetamide dioxalate; (363) N-(4-Fluorobenzyl-2-(4-isobutoxyphenyl)-N-[1-(3-pyrrolidin-1-yl-propyl)piperidin-4-yl]acetamide dioxalate; (364) N-{1-[3-(2,5-Dimethylpyrrolidin-1-yl)propyl]piperidin-4-yl}-N-(4-fluorobenzyl)-2-(4-isobutoxyphenyl)acetamide dioxalate; (365) N-(4-Fluorobenzyl)-N-{1-[3-(3-hydroxyrnethylpiperidin-1-yl)propyl]piperidin-4-yl}-2-(4-isobutoxyphenyl)acetamide dioxalate; (366) N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide oxalate; (367) N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide oxalate; (368) N-[2-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide oxalate; (369) N-(4-Fluorobenzyl)-N-{1-[3-(4-(S)-isopropyl-2-oxo-oxazolidin-3-yl)propyl]piperidin-4-yl}-2-(4-propoxyphenyl)acetamide oxalate; (370) N-{1-[2-(1,3-Dioxan-2-yl)ethyl]piperidin-4-yl}-N-(4-fluorobenzyl)-2-(4-isobutoxyphenyl)acetamide oxalate; (371) N-{1-[2-(1,3-Dioxan-2-yl)ethyl]piperidin-4-yl}-N-[2-(4-fluorophenyl)ethyl]-2-(4-isobutoxyphenyl)acetamide oxalate; (372) N-{1-[2-(1,3-Dioxan-2-yl)ethyl]piperidin-4-yl}-N-[2-(4-fluorophenyl)ethyl]-2-(4-propoxyphenyl)acetamide oxalate; (373) N-{1-[2-(1,3-Dioxan-2-yl-)ethyl]piperidin-4-yl}-N-(4-fluorobenzyl)-2-(4-propoxyphenyl)acetamide tartrate; (374) N-{1-[2-(1,3-Dioxan-2-yl)ethyl]piperidin-4-yl}-N-(4-fluorobenzyl)-W-(4-isobutoxybenzyl)carbarnide tartrate; (375) N-{1-[2-(1,3-Dioxan-2-yl)ethyl]piperidin-4-yl}-N-(4-fluorobenzyl)-2-(4-fluorophenyl)acetamide tartrate; (376) N-{1-[2-(1,3-Dioxan-2-yl)ethyl]piperidin-4-yl}-N-(4-fluorobenzyl)-2-p-tolylacetamide tartrate; (377) 2-Benzofuran-5-yl-N-{1-[2-(1,3-dioxan-2-yl)ethyl]piperidin-4-yl}-N-(4-fluorobenzyl)acetamide tartrate; (378) 2-(2,3-Dihydrobenzofuran-5-yl)-N-{1-[2-(1,3-dioxan-2-yl)ethyl]piperidin-4-yl}-N-(4-fluorobenzyl)acetamide tartrate; (379) N-{1-[2-(2,2-Dimethyl-1,3-dioxolan-4-yl)ethyl]piperidin-4-yl}-N-(4-fluorobenzyl)-2-(4-isobutoxyphenyl)acetamide tartrate; (380) N-{1-[2-(1,3-Dioxan-4-yl)ethyl]piperidin-4-yl}-N-(4-fluorobenzyl)amine; (381) N-{1-[2-(1,3-Dioxan-4-yl)ethyl]piperidin-4-yl}-N-(4-fluorobenzyl)-2-(4-isobutoxyphenyl)acetamide tartrate; (382) N-{1-[2-(1,3-Dioxan-4-yl)ethyl]piperidin-4-yl}-N(4-fluorobenzyl)-2-(4-trifluoromethylphenyl)acetamide tartrate; (383) 2-(4-Cyanophenyl)-N-{1-[2-(1,3-dioxan-4-yl)ethyl]piperidin-4-yl}-N-(4-fluorobenzyl)acetamide tartrate; (384) N-(4-Fluorobenzyl)-2-(4-isobutoxyphenyl)-N-{1-[2-(2-oxo-imidazolidin-1-yl)ethyl]piperidin-4-yl}acetamide hydrochloride; (385) 2-(4-Methoxyphenyl)-N-(4-methylbenzyl)-N-{1-[2-(2-oxo-imidazolidin-1-yl)ethyl]piperidin-4-yl}acetamide hydrochloride; (386) N-(4-Fluorobenzyl)-2-(4-isopropoxyphenyl)-N-{1-[2-(2-oxo-imidazolidin-1-yl)ethyl]piperidin-4-yl}acetamide hydrochloride; (387) N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide hydrochloride; (388) N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide hydrochloride; (389) 2-(4-Methoxyphenyl)-N-(4-methylbenzyl)-N-{1-[3-(2-oxo-2,3-dihydrobenzoimidazol-1-yl)propyl]piperidin-4-yl}-acetamide hydrochloride; (390) N-(4-Fluorobenzyl)-2-(4-isopropoxyphenyl)-N-{1-[4-(2-oxo-2,3-dihydrobenzoimidazol-1-yl)butyl]piperidin-4-yl}acetamide hydrochloride; (391) N-{1-[2-(2,4-Dioxo-1,4-dihydro-2H-quinazolin-3-ypethyl]piperidin-4-yl}-N-(4-fluorobenzyl)-2-(4-isopropoxyphenyl)acetamide hydrochloride; (392) 4-(4-Fluorobenzylamino)-piperidine-1-carboxylic acid benzyl ester; (393) N-(1-Benzyloxycarbonylpiperidin-4-yl)-N-(4-fluorobenzyl)-N′-(4-isopropoxybenzyl)carbamide; (394) N-(4-Fluorobenzyl)-N′-(4-isopropoxybenzyl)-N-piperidin-4-yl-carbamide oxalate; (395) N-{1-[2-(1,3-Dioxolan-2-yl)ethyl]piperidin-4-yl}-N-(4-fluorobenzyl)-W-(4-isopropoxy-benzyl)carbamide oxalate; (396) N-{1-[2-(1,3-Dioxolan-2-yl)ethyl]piperidin-4-yl]2-(4-methoxyphenyl)-N-(4-methylbenzyl)acetamide hydrochloride; (397) N-{1-[2-(1,3-Dioxolan-2-yl-)ethyl]piperidin-4-yl}-N-(4-fluorobenzyl)-2-(4-isobutoxyphenyl)acetamide hydrochloride; (398) N-{1-[2-(1,3-Dioxolan-2-yl)ethyl]piperidin-4-yl}-2-(4-isopropoxyphenyl)-N-(4-methylbenzyl)acetamide hydrochloride; (399) N-{1-[2-(1,3-Dioxolan-2-yl)ethyl]piperidin-4-yl}-N-(4-fluorobenzyl)-2-(4-propoxyphenyl)acetamide tartrate; (400) N-(4-Fluorobenzyl)-N′-(4-isopropoxybenzyl)-N-{1-[2-((S)-4-methyl-1,3-dioxolane-2-yl)ethyl]piperidin-4-yl}carbamide oxalate; (401) N-(4-Fluorobenzyl)-N′-(4-isopropoxybenzyl)-N-[1-(3-morpholin-4-yl-propyl)piperidin-4-yl]carbamide oxalate; (402) 2-(4-Methoxyphenyl)-N-(4-methylbenzyl)-N-[1-(2-morpholin-4-yl-ethyl)piperidin-4-yl]acetamide dihydrochloride; (403) 2-(4-Methoxyphenyl)-N-(4-methylbenzyl)-N-[1-(3-morpholin-4-ylpropyl)piperidin-4-yl]acetamide dihydrochloride; (404) N-(4-Fluorobenzyl)-2-(4-isobutoxyphenyl)-N-[1-(3-morpholin-4-ylpropyl)piperidin-4-yl]acetamide dihydrochloride; (405) N-(4-Fluorobenzyl)-2-(4-isopropoxy-phenyl)-N-[1-(3-morpholin-4-yl-propyl)piperidin-4-yl]acetamide dihydrochloride; (406) N-(4-Fluorobenzyl)-N′-(4-isopropoxybenzyl)-N-[1-(3-piperidin-1-yl-propyl)piperidin-4-yl]carbamide oxalate; (407) N-(4-Fluorobenzyl)-N′-(4-isopropoxybenzyl)-N-[1-(3-((S)-4-isopropyl-2-oxazolidinon-1-yl-propyl)piperidin-4-yl]carbamide tartrate; (408) N-(4-Fluorobenzyl)-N′-(4-isopropoxybenzyl)-N-{1-[2-(2,5,5-trimethyl-1,3-dioxan-2-yl)ethyl]}piperidin-4-yl]carbamide oxalate; (409) N-{1-[3-(1,3-Dioxolan-2-yl)propyl]piperidin-4-yl}-N-(4-fluorobenzyl)-N′-(4-isopropoxybenzyl)carbamide oxalate; (410) N-[1-(2,2-Dimethyl-1,3-dioxan-5-yl)-piperidin-4-yl]-N-(4-fluorobenzyl)-N′-(4-isopropoxybenzyl)carbamide oxalate; (411) N-(4-Fluorobenzyl)-N′-(4-isopropoxybenzyl)-N-{[2-(1-methyl pyrrolidin-2-yl)ethyl]-piperidin-4-yl}carbamide oxalate; (412) N-[1-(2,2-Dimethyl-1,3-dioxan-5-yl)piperidin-4-yl]-N-(4-fluorobenzyl)-2-(4-isobutoxyphenyl)acetamide oxalate; (413) N-[1-(1,3-Dioxan-5-yl)-piperidin-4-yl)-N-(4-fluorobenzyl)-2-(4-isobutoxyphenyl)acetamide tartrate; (414) N-[1-(2,2-Dimethyl-1,3-dioxan-5-yl)piperidin-4-yl]-N-(4-fluorobenzyl)-2-(4-fluorophenyl)acetamide tartrate; (415) N-{1-[2-(1,3-Dioxan-4-yl)ethyl]piperidin-4-yl}-N-(4-fluorobenzyl)-2-(4-fluorophenyl)acetamide tartrate: (416) N-{1-[2-(1,3-Dioxan-4-yl)ethyl]piperidin-4-yl}-N-(4-fluorobenzyl)-2-(4-trifluoromethoxyphenyl)acetamide tartrate: (417) N-{1-[2-(1,3-Dioxan-4-yl)ethyl]piperidin-4-yl}-N-(4-fluorobenzyl)-2-(4-propoxyphenyl)acetamide tartrate; (418) N-(4-Fluorobenzyl)-2-(4-isobutoxyphenyl)-N-[1-(tetrahydropyran-4-yl)piperidin-4-yl]acetamide tartrate; (419) N-(4-Fluorobenzyl)-2-(4-isobutoxyphenyl)-N-[1-(tetrahydropyran-4-ylmethyl)piperidin-4-yl]acetamide tartrate; (420) N-(4-Fluorobenzyl)-2-(4-isobutoxyphenyl)-N-{1-[2-(tetrahydropyran-4-yl)ethyl]piperidin-4-yl]acetamide tartrate; (421) N-(4-Fluorobenzyl)-2-(4-fluorophenyl)-N-[1-(tetrahydropyran-4-yl)piperidin-4-yl]acetamide tartrate; (422) N-[1-((S)-3,5-Dihydroxypentyl)piperidine-4-yl]-N-(4-fluorobenzyl)-2-(4-isobutoxyphenyl)acetamide tartrate; (423) N-{1-[2-((4S)-1,3-Dioxane-4-yl)ethyl]piperidine-4-yl}-N-(4-fluorobenzyl)-2(4-isobutoxyphenyl)acetamide tartrate; (424) N-{1-[2-(1,3-Dioxan-2-yl)ethyl]piperidin-4-yl}-N-(4-fluorobenzyl)amine; (425) 2-(4-Benzyloxyphenyl)-N-{1-[2-(1,3-dioxan-2-yl)ethyl]piperidin-4-yl}-N-(4-fluorobenzyl)acetamide tartrate; (426) N-{1-[2-(1,3-Dioxan-2-yl)ethyl]piperidin-4-yl}-N-(4-fluorobenzyl)-2-(4-hydroxyphenyl)-acetamide tartrate; (427) N-{1-[2-(1,3-Dioxan-2-yl)ethyl]piperidin-4-yl}-N-(4-fluorobenzyl)-2-(4-rnethoxyphenyl)-acetamide tartrate; (428) N-{1-[2-(1,3-Dioxan-2-yl)ethyl]piperidin-4-yl}-N-(4-fluorobenzyl)-2-(4-isopropylphenyl)-acetamide tartrate; (429) N-{1-[2-(1,3-Dioxan-2-yl)ethyl]piperidin-4-yl}-N-(4-fluorobenzyl)-2-(4-trifluorornethoxy-phenypacetamide tartrate; (430) N-{1-[2-(1,3-Dioxan-2-yl)ethyl]-piperidin-4-yl}-N-(4-fluorobenzyl)-2-(4-ethoxyphenyl)-acetamide oxalate; (431) N-{1-[2-(1,3-Dioxan-2-yl)ethyl]piperidin-4-yl}-N-(4-fluorobenzyl)-2-(4-isopropoxyphenyl)-acetamide oxalate; (432) N-{1-[2-(1,3-Dioxan-2-yl)ethyl]piperidin-4-yl}-N-(4-fluorobenzyl)-2-phenylacetamide oxalate; (433) N-{1-[2-(1,3-Dioxan-2-yl)ethyl]piperidin-4-yl}-N-(4-fluorobenzyl)-2-[4-(2-fluoroethoxy)-phenyl]acetamide oxalate; (434) N-{1-[2-(5,5-Dimethyl-1,3-dioxan-2-yl)ethyl]piperidin-4-yl}-N-(4-fluorobenzyl)-2-(4-isobutoxyphenyl)acetamide oxalate; (435) N-(4-Fluorobenzyl)-2-(4-isobutoxyphenyl)-N-{1-[2-((R)-4-methyl-1,3-dioxan-2-yl)ethyl]-piperidin-4-yl}acetamide oxalate; (436) N-(4-Fluorobenzyl)-2-(4-isobutoxyphenyl)-N-{1-[2-((S)-4-methyl-1,3-dioxolan-2-yl)ethyl]piperidin-4-yl}acetamide oxalate; (437) N-{1-[2-(4,6-Dimethyl-1,3-dioxan-2-yl)ethyl]piperidin-4-yl}-N-(4-fluorobenzyl)-2-(4-isobutoxyphenyl)acetamide oxalate; (438) N-(4-Fluorobenzyl)-N-{1-[24(S)-4-methyl-1,3-dioxolan-2-yl)ethyl]piperidin-4-yl}-2-(4-trifluoromethoxyphenyl)acetamide oxalate; (439) N-(4-Fluorobenzyl)-2-(4-isopropylphenyl)-N-{1-[2-((S)-4-methyl-1,3-dioxolan-2-yl)ethyl]-piperidin-4-yl}acetamide oxalate; (440) N-(4-Fluorobenzyl)-N-{1-[2-((R)-4-rnethyl-1,3-dioxan-2-yl)ethyl]piperidin-4-yl}-2-(4-trifluorornethoxyphenyl)acetamide oxalate; (441) N-(4-Fluorobenzyl)-2-(4-isobutoxyphenyl)-N-{1-[2-(2,5,5-trimethyl-1,3-dioxan-2-yl)ethyl]piperidin-4-yl}acetamide oxalate; (442) N-(4-Fluorobenzyl)-2-(4-isobutoxyphenyl)-N-{1-[2-(2-methyl-1,3-dioxolan-2-yl)ethyl]-piperidin-4-yl-}acetamide oxalate; (443) N-(4-Fluorobenzyl)-2-(4-isobutoxyphenyl)-N-{1-[3-(1,3-dioxolan-2-yl)propyl]piperidin-4-yl}acetamide tartrate; (444) N-(4-Fluorobenzyl)-2-(4-isobutoxyphenyl)-N-{1-(3-piperidin-1-yl-propyl)piperidin-4-yl}-acetamide dihydrochloride; (445) N-(4-Fluorobenzyl)-2-(4-isobutoxyphenyl)-N-{1-[2-(tetrahydropyran-2-yloxy)ethyl]-piperidin-4-yl}acetamide oxalate; (446) N-(4-Fluorobenzyl)-2-(4-isobutoxyphenyl)-N-{1-[3-(2-oxo-piperidin-1-yl)propyl]piperidin-4-yl}acetamide; (447) N-(4-Fluorobenzyl)-2-(4-isobutoxyphenyl)-N-{1-[3-(2-oxo-pyrrolidin-1-yl)propyl]piperidin-4-yl}acetamide hydrochloride; (448) N-(4-Fluorobenzyl)-2-(4-isobutoxyphenyl)-N-{1-[3-((R)-4-isopropyl-2-oxo-oxazolidin-3-yl)propyl]piperidin-4-yl}acetamide oxalate; (449) N-(4-Fluorobenzyl)-2-(4-isobutoxyphenyl)-N-{1-[3-(2-oxo-oxazolidin-3-yl)propyl]piperidin-4-yl}acetamide oxalate; (450) N-(4-Fluorobenzyl)-2-(4-isobutoxyphenyl)-N-{1-[3-((S)-4-methyl 2-oxo-oxazolidin-3-yl)propyl]piperidin-4-yl}acetamide tartrate; (451) N-(4-Fluorobenzyl)-2-(4-isobutoxyphenyl)-N-{1-[3-((S)-4-ethyl-2-oxo-oxazolidin-3-yl)-propyl]piperidin-4-yl}acetamide oxalate; (452) N-(4-Fluorobenzyl)-2-(4-isobutoxyphenyl)-N-{1-[2-(1,3-oxothiolan-2-yl)ethyl]piperidin-4-yl}acetamide L-tartrate; (453) 2-(4-Bromophenyl)-N-{1-[2-(1,3-dioxan-2-yl)ethyl)piperidin-4-yl}-N-(4-fluorobenzyl)-acetamide L-tartrate; (454) N-{1-[2-(1,3-Dioxan-2-yl)ethyl)piperidin-4-yl}-N-(4-fluorobenzyl)-2-(4-isobutylarnino-phenyl)acetamide L-tartrate; (455) N-{1-[2-(1,3-Dioxan-2-yl)ethyl)piperidin-4-yl}-N-(4-fluorobenzyl)-2-(4-propylarninophenyl)acetamide L-tartrate; (456) N-{1-[2-(1,3-Dioxan-2-yl)ethyl)piperidin-4-yl}-N-(4-fluorobenzyl)-2-(4-(1-nitropropyl)-phenyl)acetamide L-tartrate; (457) N-{1-[2 (1,3-Dioxan-2-yl)ethyl)piperidin-4-yl}-N-(4-fluorobenzyl)-2-[4-(2-oxopyrrolidin-1-yl)phenyl)acetamide L-tartrate; (458) N-{1-[2-(1,3-Dioxan-2-yl)ethyl)piperidin-4-yl}-N-(4-fluorobenzyl)-2-(4-isobutylsulfanylphenyl)acetamide L-tartrate; (459) N-{1-[2-(1,3-Dioxan-2-yl)ethyl)piperidin-4-yl}-N-(4-fluorobenzyl)-2-(4-iodophenyl)-acetamide L-tartrate; (460) 2-(4-Acetophenyl)-N-{1-[2-(1,3-dioxan-2-yl)ethyl)piperidin-4-yl}-N-(4-fluorobenzyl)-acetamide L-tartrate; (461) 2-[4-(1-hydroxyirninoethyl)phenyl]-N-{1-[2-(1,3-dioxan-2-yl)ethyl)piperidin-4-yl}-N-(4-fluorobenzyl)acetamide L-tartrate; (462) N-{1-[2-(1,3-Dioxan-2-yl)ethyl)piperidin-4-yl}-N-(4-fluorobenzyl)-2-(4-morpholin-4-yl-phenyl)acetamide L-tartrate; (463) N-{1-[2-(1,3-Dioxan-2-yl)ethylypiperidin-4-yl}-N-(4-fluorobenzyl)-2-(4-pyrazol-1-ylphenyl)acetamide L-tartrate; (464) N-{1-[2-(1,3-Dioxan-2-yl)-1-methylethyl]piperidin-4-yl}-N-(4-fluorobenzyl)-2-(4-isobutoxyphenyl)-acetamide L-tartrate; (465) N-{1-[2-(1,3-Dioxan-4-yl)ethyl)piperidin-4-yl}-N-(4-fluorobenzyl)-2-(4-pyrazol-1-ylphenyl)acetamide L-tartrate; (466) N-[1-((R)-3,5-Dihydroxypentyl)pipe-ridine-4-yl]-N-(4-fluorobenzyl)-2-(4-isobutoxyphenyl)acetamide tartrate; (467) N-{1-[2-((4R)-1,3-Dioxane-4-yl)ethyl]piperidine-4-yl}-N-(4-fluorobenzyl)-2-(4-isobutoxyphenyl)acetamide tartrate; (468) N-{1-[2-(1,3-Dioxan-2-yl)ethyl]piperidin-4-yl}-N-(4-fluorobenzyl)-2-[4-(1,2,4-triazol-4-yl)phenyl]acetamide L-tartrate; (469) nortriptyline; (470) duloxetine; (471) lofepramine; (472) tomoxetine; (473) 3-({1-[2-(7-methyl-5-oxo-5H)-[1,3]thiazolo[3,2-a]pyrimidin-6-yl)ethyl]-3-pyrrolidinyl}methyl)-1H-indole-5-carbonitrile hydrochloride; (474) 3-({1-[2-(6-chloro-2-oxo-2,3-dihydro-1H-indol-5-yl)ethyl]-3-pyrrolidinyl}-methyl)-1H-indole-5-carbonitrile hydrochloride; (475) moclobemide; (476) N-acetylserotonin; (477) bromfaromine; (478) beflaxozone; (479) chlorimipramine; (480) cyanimipramine; (481) cianopramine; (482) desipramine; (483) protriptyline; (484) trimipramine; (485) doxepin; (486) cyclobenzaprine; (487) 5-methoxycarbonylamino-N-acetyltryptamine; (488) amoxapine; (489) maprotiline; (490) fefazodone; (491) flesinoxan hydrochloride; (492) urapidil; (493) WY47846 (3a,4,4a,6a,7,7a-hexahydro-2-[4-[4-(2-pyrimidinyl)-1-piperazinyl]-butyl]-4,7-etheno-1H-cyclobutano[f]isoindole-1,3(2H)-dione dihydrochloride sesquihydrate); (494) SM3997 (N-[4-[4-(2-pyrimidinyl)-1-piperazinyl]butyl]-bicyclo[2.2.1]heptane-2,3-di-exo-carboximide); (495) 2-(4-(4-(2-pyrimidinyl)-1-piperazinyl-propyl)-1,2-benzoisothiazol-3-(2H)-one 1,1-dioxide hydrochloride; (496) KC9172 (3-butyl-9,9-dimethyl-7-[4-[4-[2-methoxyphenyl)-1-piperazinyl]butyl]-3,7-diazabicyclo[3,2,1]nonane-2,4,6,8-tetraone); (497) 4-(N,N-dipropylamino)-6-methoxy-1,3,4,5-tetrahydrobenz-[c,d]indole; (498) 4-[4-(N-1,2-benzisothiazol-3(2H)-one 1,1-dioxido)]butylamino-6-methoxy-1,3,4,5-tetrahydrobenz[c,d]-indole hydrochloride; (499) 5-carboxamidotryptamine; (500) N,N-dipropyl-5-carboxamidotryptamine; (501) AH25086 (3-(2-aminoethyl)-1H-indole-5-(N-methyl)acetamide); (502) GR43175 (3-(2-dimethylaminoethyl)-1H-indole-5-(N-methyl)methanesulfonamide); (503) 3-(2-[4-[2-(1,2-benzisothiazole-3(2H)-one 1,1-dioxido)]butyl]amino)ethyl-5-methoxy-1H-indole; (504) spiroxatrine; (505) MDL72832 (8-[4-(1,4-benzodioxan-2-ylmethylamino)butyl]-8-azaspiro-[4,5]decane-7,9-dione); (506) 2-[4-(1,4-benzodioxan-2-ylmethylamino)butyl]-1,2-benzisothiazol-3(2H)-one 1,1-dioxide; (507) 2-(N,N-dipropylamino)-8-hydroxy-1,2,3,4-tetrahydronaphthalene; (508) 2-{4-[2-(1,2-benzisothiazol-3(2H)-one 1,1-dioxido)]butyl}amino-8-methoxy-1,2,3,4-tetrahydronaphthalene; (509) 3-N,N-dipropylamino-5-hydroxy-thiochroman; 3-N,N-dipropylamino-5-ethoxy-thiochroman; (510) 3-N,N-dipropylamino-5-ethoxychroman; (511) 1-[2-(3-indolyl)]-ethyl-2,6-dimethylpiperidine; (512) 1-{2-[3-(5-carboxamido)indolyl]}ethyl-2,6-dimethylpiperidine; (513) RU24924 (5-methoxy-3-(1,2,3,6-tetrahydropyridin-4-yl]-1H-indole); (514) 5-methoxy-3-(1,2,3,6-tetrahydropyridin-5-yl)-1H-indole; (515) diethyl N-benzyloxycarbonyl-5-benzyloxycarbonyloxy-L-tryptophyl-L-aspartate; (516) dibenzyl N-benzyloxycarbonyl-5-hydroxy-L-tryptophanylaspartate; (517) 5-Hydroxy-L-tryptophyl-L-aspartic acid trihydrate; (518) diethyl N-benzyloxycarbonyl-5-hydroxy-L-tryptophyl-L-glutamate; (519) diethyl 5-hydroxy-L-tryptophyl-L-glutamate hydrochloride; (520) dibenzyl L-benzyloxycarbonyl-5-hydroxytryptophyl-L-glutamate; (521) 5-hydroxy-L-tryptophyl-L-glutamic acid; (522) pentachlorophenyl ester of N-benzyloxycarbonyl-5-hydroxy-L-tryptophan; (523) methyl ester of N-benzyloxycarbonyl-5-hydroxy-L-tryptophyl-L-tyrosine; (524) N-Acetyl-5-hydroxy-L-tryptophan; (525) methyl ester of N-acetyl-5-hydroxy-L-tryptophyl-L-tyrosine; (526) methyl ester of N-acetyl-5-hydroxy-L-tryptophyl-5-hydroxy-L-tryptophan; (527) 5-hydroxy-L-tryptophyl-L-alanine hydrate; (528) 5-hydroxy-L-tryptophan-L-valine; (529) 5-hydroxy-L-tryptophyl-L-leucine; (530) 5-hydroxy-L-tryptophyl-L-proline; (531) 5-hydroxy-L-tryptophyl-L-phenylalanine; (532) 5-hydroxy-L-tryptophyl-5-hydroxy-L-tryptophan; (533) 5-hydroxy-L-tryptophyl-L-tryptophan; (534) 1-(5-hydroxy)tryptophyl-L-serine; (535) 5-hydroxy-L-tryptophyl-L-arginine; (536) 5-hydroxy-L-tryptophylglycine; (537) 5-hydroxy-1-tryptophyl-gamma-aminobutyric acid; (538) 5-hydroxy-L-tryptophanamide hydrate; (539) methyl ester of 5-hydroxy-L-tryptophyl-L-histidine; (540) benzyl ester of L-5-hydroxytryptophan; (541) benzyl ester of N-benzyloxycarbonyl-5-hydroxy-L-tryptophyl-5-hydroxy-L-tryptophan; (542) 5-Hydroxy-L-tryptophyl-5-hydroxy-L-tryptophan hemihydrate; (543) 5-hydroxytryptophan inosinate; (544) theophylline salt of (DL) 5-hydroxytryptophan; (545) RU25591 (6,7,8,9-tetrahydro N, N-dimethyl 5-[4-nitrophenyl]oxy 5H-benzocyclohepten 7-amine) cis-fumarate); (546) LM5008 (4-[2-(3-indolyl)ethyl]piperidine); (547) DU24565 (6-nitro-2-(1-piperazinyl)quinoline); (548) CGP6085A (4-(5,6-dimethyl-2-benzofuranyl) piperidine hydrochloride); (549) alaprociate; (550) dibenzoxazepine; (551) deprenyl; (552) isocarboxazide; (553) furazolidone; (554) procarbazine; (555) Ro 60-0175/ORG 35030 ((S)-2-(4,4,7-trimethyl-1,4-dihydro-indeno (1,2-B) pyrrol-1-yl)-1-methyl-ethylamine) (556) Ro 60-0332/ORG 35035 ((S)-2-(Chloro-5-fluoro-indol-1-yl)-1-methylethylamine); (557) 1-[6-Chloro-5-trifluoromethyl)-2-pyridinyl]-piperazine hydrochloride; (558) 5-carboxyamidotryptamine; (559) SB 206553 (3,5-Dihydro-5-methyl-N-3-pyridinylbenzo[1,2-b:4,5-b′]dipyrrole-1(2H)-carboxamide hydrochloride); (560) ondansetron; (561) granisetron; (562) tropisetron; (563) dolasetron; (564) palonosetron; (565) trimethobenzamide; (566) risperidone; (567) clozapine; (568) azatadine; (569) cyproheptadine; (570) fenclonine; (571) chlorpromazine; (572) (313)-2,3-dihydrolysergine; (573) (313)-2,3-dihydroisolysergine; (574) (3β, 5β, 8β)-9,10-didehydro-2,3-dihydro-6-methylergoline-8-acetonitrile; (575) 25I-NBMD (2-(4-iodo-2,5-dimethoxyphenyl)-N-[(2,3-methylenedioxyphenyl)methyl]ethanamine); (576) N-(2-methoxybenzyl)-1-(8-bromo-2,3,6,7-tetrahydrobenzo[1,2-b:4,5-b′]difuran-4-yl)-2-aminoethane; (577) 5-benzyloxytryptamine; (578) 5-methoxy-7-N,N-dimethyltryptamine; (579) A372159 ((11S,16R)-3-[4-(propan-2-yloxy)-2-(trifluoromethyl)phenyl]-6-oxa-10,14-diazatetracyclo[8.6.1.0^{5,17}.0^{11,16}]heptadeca-1,3,5(17)-triene); (580) AL-34662 (14(S)-2-Aminopropyl)-1H-indazol-6-ol); (581) AL-37350A ((S)-(+)-1-(2-Aminopropyl)-8,9-dihydropyrano[3,2-e]indole); (582) AL-38022A ((S)-2-(8,9-dihydro-7H-pyrano[2,3-g]indazol-1-yl)-1-methylethylamine); (583) AS-19 ((25)-N,N-dimethyl-5-(1,3,5-trimethylpyrazol-4-yl)-1,2,3,4-tetrahydronaphthalen-2-amine); (584) alnespirone; (585) BIMU8 (N-[(1R,5S)-8-methyl-8-azabicyclo[3.2.1]oct-3-yl]-2-oxo-3-(propan-2-yl)-2,3-dihydro-1H-benzimidazole-1-carboxamide hydrochloride); (586) BMY-14802 (1-(4-fluorophenyl)-4-[4-(5-fluoropyrimidin-2-yl)piperazin-1-yl]butan-1-01); (587) BRL-54443 (3-(1-methylpiperidin-4-yl)-1H-indol-5-ol); (588) batoprazine; (589) benzylpiperazine; (590) binospirone; (591) 1-(8-bromobenzo[1,2-b;4,5-b]difuran-4-yl)-2-aminopropane); (592) CP-809,101 (2-[(3-Chlorophenyl)methoxy]-6-(1-piperazinyl)pyrazine); (593) CP-93,129 (3-(1,2,3,6-tetrahydropyrid in-4-yl)-1,4-d ihydropyrrolo[3,2-b]pyridin-5-one); (594) CP-94,253 (3-(1,2,5,6-tetrahydro-4-pyridyl)-5-propoxypyrrolo[3,2-b]pyridine); (595) CGS-12066A (4-(4-methylpiperazin-1-yl)-7-(trifluoromethyl)pyrrolo[1,2-a]quinoxaline); (596) chlorophenylbiguanide; (597) chlorphentermine; (598) dazopride; (599) dimemebfe; (600) 2,5-dimethoxy-4-bromoamphetamine; (601) 2,5-dimethoxy-4-fluoroamphetamine; (602) 2,5-dimethoxy-4-methylamphetamine; (603) EMD-386,088 (5-chloro-2-methyl-3-(1,2,3,6-tetrahydro-4-pyridinyl)-1H-indole); (604) EMDT (2-(2-ethyl-5-methoxy-1-indol-3-yl)-N,N-dimethylethanamine); (605) p-fluoropiperazine; (606) fluprazine; (607) jimscaline; (608) LY-293,284 ((4R)-6-acetyl-4-(di-n-propylamino)-1,3,4,5-tetrahydrobenz[c,d]indole); (609) lasmitidan; (610) lorcaserin; (611) 2-methyl-5-hydroxytryptamine; (612) 2-methyl-4,5-methylenedioxyamphetamine; (613) NBUMP (N-[4-[4-(2-methoxyphenyl)piperazin-1-yl]butyl]adamantane-1-carboxamide); (614) 1-(1-naphthyl)piperazine; (615) Org-37,684 ((3S)-3-[(2,3-dihydro-5-methoxy-1H-inden-4-yl)oxy]pyrrolidine); (616) PNU-22394 (6-Methyl-1,2,3,4,5,6-hexahydro-azepino[4,5-b]indole)); (617) PRX-00023 (N-(3-[4-(4-cyclohexylmethanesulfonylaminobutyl)piperazin-1-yl]phenyl)acetamide); (618) RH-34 (3-[2-(2-methoxybenzylamino)ethyl]-1H-quinazoline-2,4-dione); (619) RS56812 (N-[(3R)-1-azabicyclo[2.2.2]oct-3-yl]-2-(1-methyl-1H-indol-3-yl)-2-oxoacetamide); (620) RS67333 (1-(4-amino-5-chloro-2-methoxyphenyl)-3-(1-butyl-4-piperidinyl)-1-propanone); (621) RU24969 (5-Methoxy-3-(1,2,5,6-tetrahydro-4-pyridinyl)-1H-indole); (622) Ro60-0175 ((S)-6-Chloro-5-fluoro-1H-indole-2-propanamine); (623) TFMFIy ((2R)-1-(8-trifluoromethyl-2,3,6,7-tetrahydrobenzo[1,2-b:4,5-b′]difuran-4-yl)-2-aminoethane); (624) U92016-A ((8R)-8-(Dipropylamino)-6,7,8,9-tetrahydro-3H-benz[e]indole-2-carbonitrile) (625) VER3323 ((2S)-1-(6-bromo-2,3-dihydroindol-1-yl)propan-2-amine); (626) vilazodone; (627) WAY-181,187 (1-[(2S,5S)-4,4-difluoro-5-(hydroxymethyl)tetrahydrofuran-2-yl]pyrimidine-2,4(1H,3H)-dione); (628) WAY-208,466 (N′-[(2Z)-4-(2,4-dichlorophenyl)-3-(2-methylpropyl)-1,3-thiazol-2(3H)-ylidene]-2-(pyrazin-2-yloxy)acetohydrazide); (629) YM-348 (2S)-1-(7-ethyl-1H-furo[2,3-Mindazol-1-yl)propan-2-amine); (630) alprenolol; (631) BMY 7378 (8-(2-[4-(2-methoxyphenyl)-1-piperazinyl]ethyl)-8-azaspiro[4.5]decane-7,9-dione); (632) cyanopindolol; (633) iodocyanopindolol; (634) lezcotozan; (635) methiothepin; (636) NAN-190 (1-(2-methoxyphenyl)-4-(4-phthalimidobutyl)piperazine); (637) oxprenolol; (638) pindolol; (639) propranolol; (640) robalzotan; (641) S15535 (1-(2,3-dihydro-1,4-benzodioxin-8-yl)-4-(2,3-dihydro-1H-inden-2-yl)piperazine); (642) spiperone; (643) TFMPP; (644) UH-301 ((S)-5-fluoro-8-hydroxy-2-(dipropylamino)tetralin); (645) WAY-100,135 ((S)—N-tert-butyl-3-(4-(2-methoxyphenyl)-piperazin-1-yl)-2-phenylpropanamide); (646) WAY-100,635 (N-[2-[4-(2-methoxyphenyl)-1-piperazinyl]ethyl]-N-(2-pyridyl)cyclohexanecarboxamide); (647) mefway; (648) 5-hydroxytryptophan; (649) 5-hydroxytryptophan creatinine sulfate complex; (650) 5-methoxytryptamine; (651) 5-methoxytryptamine creatinine sulfate complex; (652) 5-HIAA (5-hydroxyindoleacetic acid); and (653) 5-HIAA (5-hydroxyindoleacetic acid) creatinine sulfate complex; and the salts, solvates, analogues, congeners, bioisosteres, hydrolysis products, metabolites, precursors, and prodrugs thereof.

12. The pharmaceutical composition of claim 1 wherein the composition comprises metformin or a salt thereof and melatonin or a salt thereof.

13. The pharmaceutical composition of claim 12 wherein the composition comprises metformin hydrochloride and melatonin.

14. The pharmaceutical composition of claim 1 wherein the composition consists essentially of the first and second agents.

15. The pharmaceutical composition of claim 1 further comprising a pharmaceutically acceptable carrier.

16. The pharmaceutical composition of claim 1, wherein the first agent is associated with a carrier substance to transport the first agent to an intended site of action.

17. The pharmaceutical composition of claim 1, wherein the second agent is associated with a carrier substance to transport the second agent to an intended site of action.

18. The pharmaceutical composition of claim 1 wherein the composition comprises the first agent and the second agent in a weight ratio of 1-1000:0.01-1.

19. The pharmaceutical composition of claim 1 wherein the composition comprises the first agent and the second agent in a weight ratio of 1-100:0.05-1.

20. The pharmaceutical composition of claim 1 wherein the composition comprises the first agent and the second agent in a weight ratio of 10-100:0.1-1.

21. A method of treating a disease or condition comprising administering a therapeutically effective quantity of the pharmaceutical composition of claim 1 to a subject that has the disease or condition or that is at risk of developing the disease or condition, in order to treat or prevent the occurrence of the disease or condition, wherein the disease or condition is selected from the group consisting of metabolic syndrome, diabetes, obesity, hypertension, cancer, AIDS, Parkinson's disease, polycystic ovarian syndrome, Alzheimer's disease, osteoporosis, sleep apnea, erectile dysfunction, McArdle disease, and a carbohydrate metabolism disorder.

22. The method of claim 21 wherein the disease or condition is selected from the group consisting of metabolic syndrome, diabetes, obesity, and hypertension.

23. The method of claim 21 wherein the disease is cancer.

24. The method of claim 21 wherein the disease or condition is selected from the group consisting of Parkinson's disease, polycystic ovarian syndrome, Alzheimer's disease, osteoporosis, sleep apnea, erectile dysfunction, McArdle disease, and a carbohydrate metabolism disorder.

25. The method of claim 21 wherein the AMPK activator is selected from the group consisting of: (1) metformin; (2) phenformin; (3) buformin; (4) AICAR; (5) a thienopyridone; (6) resveratrol; (7) nootkatone; (8) thiazole; (9) adiponectin; (10) 2-deoxyglucose; (11) AAPDs; (12) adiponectin variant polypeptides; (13) catechins; (14) trans-10, cis-12 conjugated linoleic acid; (15) a corydaline-related compound selected from the group consisting of corydaline,corlumidin, (+)-corlumidin, corypalmine, 14R-(+)-corypalmine, tetrahydropalmatine, 14R-(+)-tetrahydropalmatine, 14R,13S-(+)-corydaline, bicuculline, d-(+)-bicuculline, egenine, and +-egenine; (16) a dithiolethione; (17) an inhibitor or antagonist of DNA-dependent protein kinase catalytic subunit (DNA-PKcs); (18) a small interfering RNA (siRNA) that can inhibit the expression and/or translation of DNA-PKcs; (19) a fibrate selected from the group consisting of bezafibrate, ciprofibrate, fenofibrate, clofibrate, and gemfibrozil; (20) GW2974 (N4-(1-benzyl-1H-indazol-5-yl)-N6,N6-dimethyl-pyrido-[3,4-d]-pyrimidine-4,6-diamine); (21) honokiol; (22) leptin; (23) LKB1 (serinethreonine kinase 11); (24) obovatol (4′,5-diallyl-2,3-dihydroxybiphenyl ether); (25) a thiazolidinedione selected from the group consisting of pioglitazone and related thiazolidinediones, including rosiglitazone and rosiglitazone maleate; (26) a variant adiponectin peptide having one or more mutations at amino acid positions 109-229 of wild-type adiponectin and having at least threefold increased solubility when compared to wild-type adiponectin; (27) a butyrate compound selected from a butyrate salt and a butyrate ester; and (28) a quinoxalinedione derivative; and the salts, solvates, analogues, congeners, bioisosteres, hydrolysis products, metabolites, precursors, and prodrugs thereof.

26. The method of claim 25 wherein the AMPK activator is selected from the group consisting of metformin, phenformin, buformin, AICAR, thienopyridones, resveratrol, nootkatone, thiazole, adiponectin, thiazolidinediones, rosiglitazone, pioglitazone, dithiolethiones, and the salts, solvates, analogues, congeners, bioisosteres, hydrolysis products, metabolites, precursors, and prodrugs thereof.

27. The method of claim 21 wherein the AMPK activator is metformin or a salt thereof.

28. The method of claim 27 wherein the AMPK activator is metformin hydrochloride.

29. The method of claim 21 wherein the second agent is serotonin or a serotonin metabolite.

30. The method of claim 29 wherein the second agent is selected from the group consisting of serotonin sulfate, serotonin creatinine sulfate complex, serotonin hydrochloride, melatonin, 5-hydroxyindoleacetic acid, a salt of 5-hydroxyindoleacetic acid, a salt of melatonin, melatonin creatinine sulfate complex, and 5-hydroxyindoleacetic acid creatinine sulfate complex.

31. The method of claim 30 wherein the second agent is melatonin or a salt thereof.

32. The method of claim 21 wherein the second agent is a serotonergic compound.

33. The method of claim 32 wherein the serotonergic compound is selected from the group consisting of:
(a) serotonin transport inhibitors;
(b) serotonin receptor 2C modulators;
(c) serotonin reuptake inhibitors;
(d) serotonin and norepinephrine reuptake inhibitors;
(e) serotonin dopamine antagonists;
(f) monoamine reuptake inhibitors;
(g) pyridazinone aldose reductase inhibitors;
(h) stimulants of serotonin receptors;
(i) stimulants of serotonin synthesis;
(j) serotonin agonists;
(k) serotonin receptor 1A antagonists; and
(l) serotonin metabolites.

34. The method of claim 33 wherein the second agent is selected from the group consisting of: (1) paroxetine; (2) fluoxetine; (3) fenfluramine; (4) fluvoxamine; (5) sertraline; (6) imipramine; (7) BVT933; (8) DPCA37215; (9) IK264; (10) PNU22394 (6-methyl-1,2,3,4,5,6-hexahydro-azepino[4,5-b]indole); (11) WAY161503 (8,9-dichloro-2,3,4,4a-tetrahydro-1H-pyrazino[1,2-a]quinoxalin-5(6H)-one hydrochloride); (12) R-1065; (13) YM348 ((2S)-1-(7-ethyl-1H-furo[2,3-Mindazol-1-yl)propan-2-amine); (14) milnacipran; (15) citalopram; (16) desmethylsertraline (a metabolite of sertraline); (17) norfluoxetine; (18) desmethylcitalopram (a metabolite of citalopram); (19) escitalopram; (20) femoxetine; (21) ifoxetine; (22) cyanodothiepin; (23) litoxetine; (24) dapoxetine; (25) nefazodone; (26) cericlamine; (27) trazodone; (28) mirtazapine; (29) indalpine; (30) indeloxazine; (31) sibutramine; (32) zimeldine; (33) (+)-N-[1-[1-(4-chlorophenyl)cyclobutyl]-3-methylbutyl}-N-methylamine; (34) (−)-N-{1-[1-(4-chlorophenyl)cyclobutyl-3-methylbutyl}-N-methylamine; (35) (−)-1-[1-(4-chlorophenyl)cyclobutyl]-3-methylbutylamine; (36) (+)-N-{1-[1-(4-chlorophenyl)cyclobutyl]-3-methylbutyl}-N; (37) (−)-N-{1-[1-(4-chlorophenyl)cyclobutyl]-3-methylbutyl}-N,N-dimethylamine)N-dimethylamine; (38) venlafaxine; (39) 0-desmethylvenlafaxine (a metabolite of venlafaxine); (40) clomipramine; (41) desmethylclomipramine (a metabolite of clomipramine); (42) buspirone; (43) olanzapine; (44) ziprasidone; (45) ergoloid mesylates; (46) pergolide mesylate; (47) vitamin B1; (48) vitamin B3; (49) vitamin B6; (50) biotin; (51) S-adenosylmethionine; (52) folic acid; (53) folinic acid; (54) ascorbic acid; (55) magnesium; (56) coenzyme Q10; (57) piracetam; (58) (+)-2,5-dimethoxy-4-iodoamphetamine; (59) (+)-3,4-methylenedioxyamphetamine; (60) (+)-N-[2-[4-[2,3-dihydro-2-(hydroxymethyl)-1,4-benzodioxin-5-yl]l-piperazinyl]-4-fluorobenzamide hydrochloride; (61) (+)-norfenfluramine (a metabolite of fenfluramine); (62) (3β)-2,3-dihydrolysergene; (63) (3β)-2,3-dihydrolysergol; (64) (3β)-2,3-dihydro-methyllysergate; (65) (3β, 5β, 8β)-9,10-didehydro-2,3-dihydro-6-methyl-8-(2-pyridylthiomethyl) ergoline; (66) (3β, 5β, 8β)-9,10-didehydro-2,3-dihydro-6-methyl-8-(methylthiomethyl) ergoline; (67) (3β, 5β, 8β)-9,10-didehydro-2,3-dihydro-6-methyl-8-(phenylthiomethyl) ergoline; (68) (3β, 5β, 8β)-9,10-didehydro-2,3-dihydro-8-methyl-6-propylergoline; (69) 1-(4-bromo-2,5-dimethoxyphenyl)-2-aminopropane; (70) 1-(m-trifluoromethylphenyl)-piperazine; (71) 2-(4-(4-(2-pyrimidinyl)1-piperazinyl-propyl)-1,2-benzoisothiazol-3-(2H)-one 1,1-dioxide hydrochloride; (72) 2-methylserotonin; (73) 3β, 5β, 8β)-9,10-didehydro-2,3-dihydro-6-methylergoline-8-acetonitrile; (74) zolmitriptan; (75) 3a,4,4a,6a,7,7a-hexahydro-2-[4-[4-(2-pyrimidinyl)-11-piperazinyl]butyl]-4,7-etheno-1H-cyclobutanoisoindole-1,3(2H)-dione dihydrochloride sesquihydrate; (76) 3-butyl-9,9-dimethyl-7-[4-[4-[2-methoxyphenyl) 1-piperazinyl]butyl]-3,7-diazabicyclo[3,2,1]nonane-2,4,6,8-tetraone; (77) 4,4-dimethyl-1-[4-[4-(2-pyrimidinyl)-1-piperazinyl]butyl]2,6-piperidinedione hydrochloride; (78) 5-hydroxy-L-tryptophan; (79) 5-methoxy-N,N-dimethyltryptamine; (80) 6-[[3-[4[o-methoxyphenyl]-1-piperazinyl]propyl]-amino]-1,3-dimethyluracil; (81) 8-[4-N-[4-(2-pyrimidinyl)-1-piperazinyl]-butyl]-8-azaspiro[4.5]-decane-7,9-dione hydrochloride; (82) 8-hydroxy-2-(di-n-propylamino)tetralin (8-OH-DPAT); (83) alniditan; (84) almotriptan; (85) 2-aminotetralin; (86) bifeprunox; (87) gepirone; (88) BW723C86 (1-[5(2-thienylmethoxy)-1H-3-indolyl[propan-2-amine hydrochloride); (89) cisapride; (90) dihydroergotamine; (91) D-lysergic acid diethylamide; (92) donitriptan; (93) eletriptan; (94) frovatriptan; (95) tegaserod; (96) ipsapirone; (97) L694247 (2-[5-[3-(4-methylsulphonylamino)benzyl-1,2,4-oxadiazol-5-yl]-1H-indol-3yl]ethanamine); (98) cinitapride; (99) lesopitron; (100) MCPP (m-chlorophenylpiperazine); (101) methysergide; (102) metoclopramide; (103) MK-212 (6-chloro-2-(1-piperazinyl)pyrazine hydrochloride); (104) mosapride; (105) N,N-dimethyl-5-methoxytryptamine; (106) N,N-dimethyltryptamine; (107) N-[4-[4-(2-pyrimidinyl)-1-piperazinyl]butylbicyclo[2.2.1]heptane-2,3-di-oxo-carboximide; (108) naratriptan; (109) norcisapride; (110) phentermine; (111) quipazine; (112) prucalopride; (113) rauwolscine; (114) repinotan; (115) rizatriptan; (116) sumatriptan; (117) tandospirone; (118) 1-methyl-4-phenyl-1,2,3,6-tetrahydropyridine; (119) tiaspirone; (120) trifluoromethylphenylpiperazine; (121) L-tryptophan; (122) xaliproden; (123) yohimbine; (124) zacopride; (125) zalospirone (126) mianserin; (127) setiptiline; (128) adatanserin; (129) altanserin; (130) benanserin; (131) blonanserin; (132) butanserin; (133) cinanserin; (134) eplivanserin; (135) flibanserin (136) glemanserin; (137) iferanserin; (138) ketanserin; (139) lidanserin; (140) pelanserin; (141) pruvanserin; (142) ritanserin; (143) seganserin; (144) tropanserin; (145) iloperidone; (146) sertindole; (147) EMR-62218; (148) asenapine; (149) zotepine; (150) ocaperidone; (151) APD125; (152) AVE8488; (153) pimavanserin; (154) isocarboxazid; (155) phenelzine; (156) tranylcypromine; (157) amitriptyline; (158) clomipramine; (159) N-(1-(1-methylethyl)piperidin-4-yl)-N-((4-methylphenyl)methyl)-4-methoxyphenylacetamide; (160) N-(1-(2,2-dimethylethyl)piperidin-4-yl)-N-((4-methylphenyl)methyl)-4-methoxyphenylacetamide; (161) N-(1-pentylpiperidin-4-yl)-N-((4-methylphenyl)methyl)-4-methoxyphenylacetamide; (162) N-(1-hexylpiperidin-4-yl)-N-((4-methylphenyl)methyl)-4-methoxyphenylacetamide; (163) N-(1-cyclohexylpiperidin-4-yl)-N-((4-methylphenyl)methyl)-4-methoxyphenylacetamide; (164) N-(1-cyclopentylpiperidin-4-yl)-N-((4-methylphenyl)methyl)-4-methoxyphenylacetamide; (165) N-(1-cyclobutylpiperidin-4-yl)-N-((4-methylphenyl)methyl)-4-methoxyphenylacetamide; (166) N-(1-cyclopropylpiperidin-4-yl)-N-((4-methylphenyl)methyl)-4-methoxyphenylacetamide; (167) N-(1-(cyclopentylmethyl)piperidin-4-yl)-N-((4-methylphenyl)methyl)-4-methoxyphenylacetamide; (168) N-(1-(cyclobutylmethyl)piperidin-4-yl)-N-((4-methylphenyl)methyl)-4-methoxyphenylacetamide; (169) N-(1-(cyclopropylmethyl)piperidin-4-yl)-N-((4-methylphenyl)methyl)-4-methoxyphenylacetamide; (170) N-(1-(2-hydroxyethyl)piperidin-4-yl)-N-((4-methylphenyl)methyl)-4-methoxyphenylacetamide; (171) N-(1-(3-hydroxypropyl)piperidin-4-yl)-N-((4-methylphenyl)methyl)-4-methoxyphenylacetamide; (172) N-((4-Methylphenyl)methyl)-N-(piperidin-4-yl)-N′-phenylmethylcarbamide; (173) N-((4-Methylphenyl)methyl)-N-(1-(2-methylpropyl)piperidin-4-yl)-N′-phenylmethylcarbamide; (174) N-(1-((2-Bromophenyl)methyl)piperidin-4-yl)-N-((4-methylphenyl)methyl)-N′-phenylmethylcarbamide; (175) N-(1-((4-Hydroxy-3-methoxyphenyl)methyl)piperidin-4-yl)-N-((4-methylphenyl) methyl)-N′-phenylmethylcarbamide; (176) N-(1-((5-Ethylthien-2-yl)methyl)piperidin-4-yl)-N-((4-methylphenyl)methyl)-N′-phenylmethylcarbamide; (177) N-(1-(Imidazol-2-ylmethyl)piperidin-4-yl)-N-((4-methylphenyl)methyl)-N′-phenylmethylcarbamide; (178) N-(1-(Cyclohexylmethyl)piperidin-4-yl)-N-((4-methylphenyl)methyl)-N′-phenylmethylcarbamide; (179) N-(1-((4-Fluorophenyl)methyl)piperidin-4-yl)-N-((4-methylphenyl)methyl)-N′-phenylmethylcarbamide; (180) N-((4-Methylphenyl)methyl)-N-(piperidin-4-yl)-4-methoxyphenylacetamide; (181) N-((4-Methylphenyl)methyl)-N-(1-methylpiperidin-4-yl)-4-methoxyphenylacetamide; (182) N-(1-Ethylpiperidin-4-yl)-N-((4-methylphenyl)methyl)-4-methoxyphenylacetamide; (183) N-((4-Methylphenyl)methyl)-N-(1-propylpiperidin-4-yl)-4-methoxyphenylacetamide; (184) N-(1-Butylpiperidin-4-yl)-N-((4-methylphenyl)methyl)-4-methoxyphenylacetamide; (185) N-(1-(3,3-Dimethylbutyl)piperidin-4-yl)-N-((4-methylphenyl)methyl)-4-methoxyphenylacetamide; (186) N-(1-(Cyclohexylmethyl)piperidin-4-yl)-N-((4-methylphenyl)methyl)-4-methoxyphenylacetamide; (187) N-((4-Methylphenyl)methyl)-N-(1-(2-methylpropyl)piperidin-4-yl)-4-methoxyphenylacetamide; (188) N-((4-Methylphenyl)methyl)-N-(1-((4-methylphenyl)methyl)piperidin-4-yl)-4-methoxyphenylacetamide; (189) N-(1-((4-Hydroxyphenyl)methyl)piperidin-4-yl)-N-((4-methylphenyl)methyl)-4-methoxyphenylacetamide; (190) N-(1-((2-Hydroxyphenyl)methyl)piperidin-4-yl)-N-((4-methylphenyl)methyl)-4-methoxyphenylacetamide; (191) N-(3-Phenylpropyl)-N-(piperidin-4-yl)-4-methoxyphenylacetamide; (192) N-(2-Phenylethyl)-N-(piperidin-4-yl)-4-methoxyphenylacetamide; (193) N-((2-Methoxyphenyl)methyl)-N-(piperidin-4-yl)-4-methoxyphenylacetamide; (194) N-((2-Chlorophenyl)methyl)-N-(piperidin-4-yl)-4-methoxyphenylacetamide; (195) N-((3,4-Di-methoxyphenyl)methyl)-N-(piperidin-4-yl)-4-methoxyphenylacetamide; (196) N-((4-Fluorophenyl)methyl)-N-(piperidin-4-yl)-4-methoxyphenylacetamide; (197) N-((2,4-Di-chlorophenyl)methyl)-N-(piperidin-4-yl)-4-methoxyphenylacetamide; (198) N-((3-Methylphenyl)methyl)-N-(piperidin-4-yl)-4-methoxyphenylacetamide; (199) N-((3-Bromophenyl)methyl)-N-(piperidin-4-yl)-4-methoxyphenylacetamide; (200) N-(1-(Phenylmethyl)piperidin-4-yl)-N-(3-phenyl-2-propen-1-yl)-4-methoxyphenylacetamide; (201) N-((4-Methylphenyl)methyl)-N-(1-piperidin-4-yl)-phenylacetamide; (202) N-((4-Methylphenyl)methyl)-N-(1-piperidin-4-yl)-3-phenylpropionamide; (203) N-((4-Methylphenyl)methyl)-N-(1-piperidin-4-yl)-(phenylthio)acetamide; (204) N-((4-Methylphenyl)methyl)-N-(1-piperidin-4-yl)-phenoxyacetamide; (205) N-((4-Methylphenyl)methyl)-N-(1-piperidin-4-yl)-(4-chlorophenoxy)acetamide; (206) N-((4-Methylphenyl)methyl)-N-(1-piperidin-4-yl)-3-methoxyphenylacetamide; (207) N-((4-Methylphenyl)methyl)-N-(1-piperidin-4-yl)-4-fluorophenylacetamide; (208) N-((4-Methylphenyl)methyl)-N-(1-piperidin-4-yl)-2,5-di-methoxyphenylacetamide; (209) N-((4-Methylphenyl)methyl)-N-(1-piperidin-4-yl)-4-chlorophenylacetamide; (210) N-((4-Methylphenyl)methyl)-N-(1-(phenylmethyl)pyrrolidin-3-yl)-N′-phenylmethylcarbamide; (211) N-((4-Methylphenyl)methyl)-N-(1-(phenylmethyl)pyrrolidin-3-yl)-4-methoxyphenylacetamide; (212) 2-(4-methoxyphenyl)-N-(4-methylbenzyl)-N-(piperidin-4-yl)acetamide; (213) 2-(4-methoxyphenyl)-N-(4-methylbenzyl)-N-(1-methylpiperidin-4-yl)acetamide; (214) 2-(4-methoxyphenyl)-N-(4-methylbenzyl)-N-(1-ethylpiperidin-4-yl)acetamide; (215) 2-(4-methoxyphenyl)-N-(4-chlorbenzyl)-N-(1-ethylpiperidin-4-yl)acetamide; (216) 2-(4-methoxyphenyl)-N-(4-chlorbenzyl)-N-(1-isopropylpiperidin-4-yl)acetamide; (217) 2-(4-methoxyphenyl)-N-(4-chlorobenzyl)-N-(piperidin-4-yl)acetamide; (218) 2-(4-methoxyphenyl)-N-(4-chlorobenzyl)-N-(1-cyclopentylpiperidin-4-yl)acetamide; (219) 2-(4-methoxyphenyl)-N-(4-chlorbenzyl)-N-(1-isopropylpiperidin-4-yl)acetamide; (220) 2-(phenyl)-N-(4-trifluoromethylbenzyl)-N-(1-methylpiperidin-4-yl)acetamide; (221) 2-(4-fluorophenyl)-N-(4-trifluoromethylbenzyl)-N-(1-methylpiperidin-4-yl)acetamide; (222) 2-(4-Methoxyphenyl)-N-(4-trifluoromethylbenzyl)-N-(1-methylpiperidin-4-yl)acetamide; (223) 2-(4-Trifluoromethylphenyl)-N-(4-trifluoromethylbenzyl)-N-(1-methylpiperidin-4-yl)acetamide; (224) 2-(4-Fluorophenyl)-N-(4-fluorobenzyl)-N-(1-methylpiperidin-4-yl)acetamide; (225) 2-(4-Methoxyphenyl)-N-(4-fluorobenzyl)-N-(1-methylpiperidin-4-yl)acetamide; (226) 2-(phenyl)-N-(4-fluorobenzyl)-N-(1-methylpiperidin-4-yl)acetamide; (227) 2-(4-Trifluoromethylphenyl)-N-(4-fluorobenzyl)-N-(1-methylpiperidin-4-yl)acetamide; (228) 2-(4-trifluoromethylphenyl)-N-[4-(methoxycarbonyl)benzyl]-N-(1-methylpiperidin-4-yl)acetamide; (229) 2-Phenyl-N-[4-(methoxycarbonyl)benzyl]-N-(1-methylpiperidin-4-yl)acetamide; (230) 2-(4-Chlorophenyl)-N-[4-(methoxycarbonyl)benzyl]-N-(1-methylpiperidin-4-yl) acetamide; (231) 2-(4-Methoxyphenyl)-N-[4-(methoxycarbonyl)benzyl]-N-(1-methylpiperidin-4-yl)acetamide; (232) 2-(4-trifluoromethylphenyl)-N-[4-(methoxycarbonyl)benzyl]-N-(1-methylpiperidin-4-yl)acetamide; (233) 2-Phenyl-N-[4-(methoxycarbonyl)benzyl]-N-(1-methylpiperidin-4-yl)acetamide; (234) 2-(4-Chlorophenyl)-N-[4-(methoxycarbonyl)benzyl]-N-(1-methylpiperidin-4-yl) acetamide; (235) 2-(4-Methoxyphenyl)-N-[4-(methoxycarbonyl)benzyl]-N-(1-methylpiperidin-4-yl)acetamide; (236) 2-(4-methoxyphenyl)-N-(4-methylbenzyl)-N-[1-(4-chloromethyl-2-thiazolylmethyl)piperidin-4-yl]acetamide; (237) 2-(4 methoxyphenyl)-N-(4-methylbenzyl)-N-{1-[3-(1,3-dihydro-2H-benzimidazol-2-on-1-yl)propyl]piperidin-4-yl}acetamide; (238) 2-(4-methoxyphenyl)-N-(2-4(fluorophenyl)ethyl)-N-(1-methylpiperidin-4-yl)acetamide; (239) 2-(4-methoxyphenyl)-N-[2-(2,5-dimethoxyphenyl)ethyl]-N-(1-methylpiperidin-4-yl)acetamide; (240) 2-(4-methoxyphenyl)-N-[2-(2,4-dichlorophenyl)ethyl]-N-(1-methylpiperidin-4-yl)acetamide; (241) 2-(4-methoxyphenyl)-N-[2-(3-chlorophenyl)ethyl]-N-(1-methylpiperidin-4-yl)acetamide; (242) 2-(4-methoxyphenyl)-N-[2-(4-methoxyphenyl)ethyl]-N-(1-methylpiperidin-4-yl) acetamide; (243) 2-(4-methoxyphenyl)-N-[2-(3-fluorophenyl)ethyl]-N-(1-methylpiperidin-4-yl)acetamide; (244) 2-(4-ethoxyphenyl)-N-[2-(4-fluorophenethyl]-N-(1-methylpiperidin-4-yl)acetamide; (245) 2-(4-ethoxyphenyl)-N-(4-fluorobenzyl)-N-(1-methylpiperidin-4-yl)acetamide; (246) 2-(4-methoxyphenyl)-N-(4-methylbenzyl)-N-{1-[2-(2-hydroxyethoxy)ethyl]piperidin-4-yl}acetamide; (247) 2-(4-methoxyphenyl)-N-(4-methylbenzyl)-N-[1-((2-chloro-5-thienyl)methyl) piperidin-4-yl]acetamide; (248) 2-(4-methoxyphenyl)-N-(4-methylbenzyl)-N-[1-(2-(imidazolidinon-1-yl)ethyl)piperidin-4-yl]acetamide; (249) 2-(4-methoxyphenyl)-N-(4-methylbenzyl)-N-{1-[2-(2,4(1H,3H)quinazolinedion-3-yl)ethyl]piperidin-4-yl}acetamide; (250) 2-(4-methoxyphenyl)-N-(4-methylbenzyl)-N-{1-[2-(1,3-dioxolan-2-yl)ethyl]piperidin-4-yl}acetamide; (251) 2-(4-methoxyphenyl)-N-(4-methylbenzyl)-N-{1-[2-(3-indolyl)ethyl]piperidin-4-yl}acetamide; (252) 2-(4-methoxyphenyl)-N-(4-methylbenzyl)-N-{1-[3-(1,2,4-triazol-1-yl)propyl]piperidin-4-yl}acetamide; (253) 2-(4-methoxyphenyl)-N-(4-methylbenzyl)-N-[1-(5-benzofurazanylmethyl)piperidin-4-yl]acetamide; (254) 2-(4-methoxyphenyl)-N-(4-methylbenzyl)-N-[1-(5-chlorobenzo[b]thien-3-ylmethyl)piperidin-4-yl]acetamide; (255) 2-(4-methoxyphenyl)-N-(4-methylbenzyl)-N-[1-(5-phenyl-1,2,4-oxadiazol-3-ylmethyl)piperidin-4-yl]acetamide; (256) 2-(4-Chlorophenyl)-N-(4-methylbenzyl)-N-(1-isopropylpiperidin-4-yl)-acetamide; (257) 2-(4-Chlorophenyl)-N-(4-methylbenzyl)-N-(1-ethylpiperidin-4-yl)-acetamide; (258) 2-Phenyl-N-(4-methylbenzyl)-N-(1-methylpiperidin-4-yl)-acetamide; (259) 2-(4-Chlorophenyl)-N-(4-methylbenzyl)-N-(1-methylpiperidin-4-yl)-acetamide; (260) 2-(4-Chlorophenyl)-N-(4-methylbenzyl)-N-(1-cyclopentylpiperidin-4-yl)-acetamide; (261) 2-(4-Fluorophenyl)-N-(4-methylbenzyl)-N-(1-methylpiperidin-4-yl)-acetamide; (262) 2-(4-Chlorophenyl)-N-(4-methylbenzyl)-N-(1-(2-hydroxyethyl)-piperidin-4-yl)-acetamide; (263) 2-(4-Chlorophenyl)-N-(4-methylbenzyl)-N-(1-cyclobutylpiperidin-4-yl)-acetamide; (264) 2-(4-Methoxyphenyl)-N-(4-methylbenzyl)-N-(1-cyclobutylpiperidin-4-yl)-acetamide; (265) 2-(4-Methoxyphenyl)-N-(4-methylbenzyl)-N-(tropin-4-yl)-acetamide; (266) N-(4-Methylbenzyl)-N-(1-methylpiperidin-4-yl)-N′-benzyl-carbamide; (267) N-(4-Methylbenzyl)-N-(1-methylpiperidin-4-yl)-N′-phenyl-carbamide; (268) N-Phenethyl-N-(1-methylpiperidin-4-yl)-N′-benzyl-carbamide; (269) 2-Phenyl-N-(4-methoxybenzyl)-N-(1-methylpiperidin-4-yl)-acetamide; (270) 2-(4-Trifluoromethylphenyl)-N-(4-methoxybenzyl)-N-(1-methylpiperidin-4-yl)-acetamide (271) 2-(4-Fluorophenyl)-N-(4-methoxybenzyl)-N-(1-methylpiperidin-4-yl)-acetamide; (272) 2-(4-Methoxyphenyl)-N-(4-methoxybenzyl)-N-(1-methylpiperidin-4-yl)-acetamide; (273) 2-(4-Methylphenyl)-N-(4-chlorobenzyl)-N-(1-methylpiperidin-4-yl)-acetamide; (274) 2-(4-Hydroxyphenyl)-N-(4-methylbenzyl)-N-(1-methylpiperidin-4-yl)-acetamide; (275) N-Phenethyl-N-(1-methylpiperidin-4-yl)-N′-phenyl-carbamide; (276) N-(3-Phenylpropyl)-N-(1-methylpiperidin-4-yl)-N′-benzyl-carbamide; (277) N-(3-Phenylpropyl)-N-(1-methylpiperidin-4-yl)-N′-phenyl-carbamide; (278) 2-(4-Methoxyphenyl)-2,2-ethylene-N-(4-methylbenzyl)-N-(1-methylpiperidin-4-yl)acetamide; (279) 2-(4-Methoxyphenyl)-N-alpha-methylbenzyl-N-(1-methylpiperidin-4-yl)acetamide; (280) 2-(4-Methoxyphenyl)-N-(4-methylbenzyl)-N-(3-tropen-4-ypacetamide; (281) 2-Phenyl-2-ethyl-N-(4-methylbenzyl)-N-(1-methylpiperidin-4-yl)acetamide; (282) N-Phenethyl-N-(4-methylbenzyl)-N-(1-methylpiperidin-4-yl)-amine; (283) 2-(4-Methoxyphenyl)-N-(1-indanyl)-N-(1-methylpiperidin-4-yl)acetamide; (284) N-(4-Methylbenzyl)-N-(1-methylpiperidin-4-yl)-N′-(4-methoxybenzyl)-carbamide; (285) 2-(3,4-dimethoxyphenyl)-N-(4-methylbenzyl)-N-(1-methylpiperidin-4-yl)acetamide; (286) 2-(3,4-Methylenedioxyphenyl)-N-(4-methylbenzyl)-N-(1-methylpiperidin-4-yl)acetamide; (287) 2-(4-Methoxyphenyl)-N-(4-methylbenzyl)-N-(1-t-butylpiperidin-4-yl)-acetamide; (288) N-(4-Methylbenzyl)-N-(1-methylpiperidin-4-yl)-N′-phenethyl-carbamide; (289) N-Phenethyl-N-(1-methylpiperidin-4-yl)-N′-phenethyl-carbamide; (290) N-(4-Methylbenzyl)-N-(1-t-butylpiperidin-4-yl)-N′-(4-methoxybenzyl)-carbamide; (291) 2-(4-Ethoxyphenyl)-N-(4-methylbenzyl)-N-(1-methylpiperidin-4-yl)acetamide; (292) 2-(4-Butoxyphenyl)-N-(4-methylbenzyl)-N-(1-methylpiperidin-4-yl)acetamide; (293) 2-(4-i-Propoxyphenyl)-N-(4-methylbenzyl)-N-(1-methylpiperidin-4-yl)acetamide; (294) 2-(4-t-Butoxyphenyl)-N-(4-methylbenzyl)-N-(1-methylpiperidin-4-yl)acetamide; (295) 2-(4-Butoxyphenyl)-N-(4-fluorobenzyl)-N-(1-methylpiperidin-4-yl)acetamide; (296) 2-(4-Propoxyphenyl)-N-(4-fluorobenzyl)-N-(1-methylpiperidin-4-yl)acetamide; (297) 2-(4-i-Propoxyphenyl)-N-(4-fluorobenzyl)-N-(1-methylpiperidin-4-yl)acetamide; (298) 2-(4-t-Butoxyphenyl)-N-(4-fluorobenzyl)-N-(1-methylpiperidin-4-yl)acetamide; (299) 4-(4-Fluorobenzyl)-3-(4-methoxybenzyl)-8-methyl-1-oxa-3,8-diaza-spiro[4.5]decan-2-one; (300) 3-(4-Ethoxybenzyl)-4-(4-fluorobenzyl)-8-methyl-1-oxa-3,8-diaza-spiro[4.5]decan-2-one; (301) 4-(4-Fluorobenzyl)-8-methyl-3-(4-propoxybenzyl)-1-oxa-3,8-diaza-spiro[4.5]decan-2-one; (302) 3-(4-Cyclopropylmethoxybenzyl)-4-(4-fluorobenzyl)-8-methyl-1-oxa-3,8-diaza-spiro[4.5]decan-2-one; (303) 4-(4-Fluorobenzyl)-3-(4-isopropoxybenzyl)-8-methyl-1-oxa-3,8-diaza-spiro[4.5]decan-2-one; (304) 3-(4-Butoxybenzyl)-4-(4-fluorobenzyl)-8-methyl-1-oxa-3,8-diaza-spiro[4.5]decan-2-one; (305) 4-(4-Fluorobenzyl)-3-(4-isobutoxybenzyl)-8-methyl-1-oxa-3,8-diaza-spiro[4.5]decan-2-one; (306) 3-(4-Difluoromethoxybenzyl)-4-(4-fluorobenzyl)-8-methyl-1-oxa-3,8-diaza-spiro[4.5]decan-2-one; (307) 4-(4-Fluorobenzyl)-8-methyl-3-(4-trifluoromethoxybenzyl)-1-oxa-3,8-diaza-spiro[4.5]decan-2-one; (308) 4-(4-Fluorobenzyl)-8-methyl-3-(4-pentoxybenzyl)-1-oxa-3,8-diaza-spiro[4.5]decan-2-one; (309) 8-Ethyl-4-(4-fluorobenzyl)-3-(4-isobutoxybenzyl)-1-oxa-3,8-diaza-spiro[4.5]decan-2-one; (310) 4-(4-Fluorobenzyl)-3-(4-isobutoxybenzyl)-8-isopropyl-1-oxa-3,8-diaza-spiro[4.5]decan-2-one; (311) 8-Cyclopropylmethyl-4-(4-fluorobenzyl)-3-(4-isobutoxybenzyl)-1-oxa-3,8-diaza-spiro[4.5]decan-2-one; (312) 8-Cyclohexylmethyl-4-(4-fluorobenzyl)-3-(4-isobutoxybenzyl)-1-oxa-3,8-diaza-spiro[4.5]decan-2-one; (313) 8-Cyclopentyl-4-(4-fluorobenzyl)-3-(4-isobutoxybenzyl)-1-oxa-3,8-diaza-spiro[4.5]decan-2-one; (314) 4-(4-Fluorobenzyl)-3-(4-isobutoxybenzyl)-8-(3-morpholin-4-yl-propyl)-1-oxa-3,8-diaza-spiro[4.5]decan-2-one; (315) 8-(2-[1,3]Dioxolan-2-yl-ethyl)-4-(4-fluorobenzyl)-3-(4-isobutoxybenzyl)-1-oxa-3,8-diaza-spiro[4.5]decan-2-one; (316) 4-(4-Fluorobenzyl)-3-(4-isobutoxybenzyl)-8-[2-(2-oxo-imidazolidin-1-yl)-ethyl]-1-oxa-3,8-diaza-spiro[4.5]decan-2-one; (317) 4-(4-Fluorobenzyl)-3-(4-isobutoxybenzyl)-8-[3-(2-oxo-2,3-dihydro-benzoimid azol-1-yl)-propyl]-1-oxa-3,8-diaza-spiro[4.5]decan-2-one; (318) 4-(4-Fluorobenzyl)-3-(4-isobutoxybenzyl)-8-(2-methyl-thiazol-4-yl-methyl)-1-oxa-3,8-diaza-spiro[4.5]decan-2-one; (319) 4-(4-Chlorobenzyl)-3-(4-isobutoxybenzyl)-8-methyl-1-oxa-3,8-diaza-spiro[4.5]decan-2-one; (320) 8-Ethyl-4-(4-chlorobenzyl)-3-(4-isobutoxybenzyl)-1-oxa-3,8-diaza-spiro[4.5]decan-2-one; (321) 4-(4-Chlorobenzyl)-3-(4-isobutoxybenzyl)-8-isopropyl-1-oxa-3,8-diaza-spiro[4.5]decan-2-one; (322) 8-Cyclopropylmethyl-4-(4-chlorobenzyl)-3-(4-isobutoxybenzyl)-1-oxa-3,8-diaza-spiro[4.5]decan-2-one; (323) 8-Cyclohexylmethyl-4-(4-chlorobenzyl)-3-(4-isobutoxybenzyl)-1-oxa-3,8-diaza-spiro[4.5]decan-2-one; (324) 8-(2-[1,3]Dioxolan-2-yl-ethyl)-4-(4-chlorobenzyl)-3-(4-isobutoxybenzyl)-1-oxa-3,8-diaza-spiro[4.5]decan-2-one; (325) 4-(4-Chlorobenzyl)-3-(4-isobutoxybenzyl)-8-[2-(2-oxo-imidazolidin-1-yl)-ethyl]-1-oxa-3,8-diaza-spiro[4.5]decan-2-one; (326) 3-(4-Difluoromethoxybenzyl)-4-(4-fluorobenzyl)-8-methyl-1-oxa-3,8-diaza-spiro[4.5]decan-2-one; (327) 3-(4-Difluoromethoxybenzyl)-8-ethyl-4-(4-fluorobenzyl)-1-oxa-3,8-diaza-spiro[4.5]decan-2-one; (328) 3-(4-Difluoromethoxybenzyl)-4-(4-fluorobenzyl)-8-isopropyl-1-oxa-3,8-diaza-spiro[4.5]decan-2-one; (329) 8-Cyclopropylmethyl-3-(4-difluoromethoxybenzyl)-4-(4-fluorobenzyl)-1-oxa-3,8-diaza-spiro[4.5]decan-2-one; (330) 8-Cyclohexylmethyl-3-(4-difluoromethoxybenzyl)-4-(4-fluorobenzyl)-1-oxa-3,8-diaza-spiro[4.5]decan-2-one; (331) 3-(4-Difluoromethoxybenzyl)-8-(2-[1,3]dioxolan-2-yl-ethyl)-4-(4-fluorobenzyl)-1-oxa-3,8-diaza-spiro[4.5]decan-2-one; (332) 3-(4-Difluoromethoxybenzyl)-4-(4-fluorobenzyl)-8-[2-(2-oxo-imidazolidin-1-yl)-ethyl]-1-oxa-3,8-diaza-spiro[4.5]decan-2-one; (333) 8-Ethyl-4-(4-fluorobenzyl)-3-(4-trifluoromethoxybenzyl)-1-oxa-3,8-diaza-spiro[4.5]decan-2-one; (334) 4-(4-Fluorobenzyl)-8-isopropyl-3-(4-trifluoromethoxybenzyl)-1-oxa-3,8-diaza-spiro[4.5]decan-2-one; (335) 8-Cyclopropylmethyl-4-(4-fluorobenzyl)-3-(4-trifluoromethoxybenzyl)-1-oxa-3,8-diaza-spiro[4.5]decan-2-one; (336) 8-Cyclohexylmethyl-4-(4-fluorobenzyl)-3-(4-trifluoromethoxybenzyl)-1-oxa-3,8-diaza-spiro[4.5]decan-2-one; (337) 8-Cyclopentyl-4-(4-fluorobenzyl)-3-(4-trifluoromethoxybenzyl)-1-oxa-3,8-diaza-spiro[4.5]decan-2-one; (338) 8-(2-[1,3]Dioxolan-2-yl-ethyl)-4-(4-fluorobenzyl)-3-(4-trifluoromethoxybenzyl)-1-oxa-3,8-diaza-spiro[4.5]decan-2-one; (339) 4-(4-Fluorobenzyl)-8-[2-(2-oxo-imidazolidin-1-yl)-ethyl]-3-(4-trifluoromethoxybenzyl)-1-oxa-3,8-diaza-spiro[4.5]decan-2-one; (340) 8-Ethyl-4-(4-fluorobenzyl)-3-(4-propoxybenzyl)-1-oxa-3,8-diaza-spiro[4.5]decan-2-one; (341) 4-(4-Fluorobenzyl)-8-isopropyl-3-(4-propoxybenzyl)-1-oxa-3,8-diaza-spiro[4.5]decan-2-one; (342) 8-Cyclopropylmethyl-4-(4-fluorobenzyl)-3-(4-propoxybenzyl)-1-oxa-3,8-diaza-spiro[4.5]decan-2-one; (343) 8-Cyclohexylmethyl-4-(4-fluorobenzyl)-3-(4-propoxybenzyl)-1-oxa-3,8-diaza-spiro[4.5]decan-2-one; (344) 8-Cyclopentyl-4-(4-fluorobenzyl)-3-(4-propoxybenzyl)-1-oxa-3,8-diaza-spiro[4.5]decan-2-one; (345) 8-(2-[1,3]Dioxolan-2-yl-ethyl)-4-(4-fluorobenzyl)-3-(4-propoxybenzyl)-1-oxa-3,8-diaza-spiro[4.5]decan-2-one; (346) 4-(4-Fluorobenzyl)-8-[2-(2-oxo-imidazolidin-1-yl)-ethyl]-3-(4-propoxybenzyl)-1-oxa-3,8-diaza-spiro[4.5]decan-2-one; (347) 3-(4-Cyclopropylmethoxybenzyl)-8-ethyl-4-(4-fluorobenzyl)-1-oxa-3,8-diaza-spiro[4.5]decan-2-one; (348) 3-(4-Cyclopropylmethoxybenzyl)-4-(4-fluorobenzyl)-8-isopropyl-1-oxa-3,8-diaza-spiro[4.5]decan-2-one; (349) 3-(4-Cyclopropylmethoxybenzyl)-8-cyclopropylmethyl-4-(4-fluorobenzyl)-1-oxa-3,8-diaza-spiro[4.5]decan-2-one; (350) 3-(4-Cyclopropylmethoxybenzyl)-8-(2-[1,3]dioxolan-2-yl-ethyl)-4-(4-fluorobenzyl)-1-oxa-3,8-diaza-spiro[4.5]decan-2-one; (351) 3-(4-Cyclopropylmethoxybenzyl)-4-(4-fluorobenzyl)-8-[2-(2-oxo-imidazolidin-1-yl)-ethyl]-1-oxa-3,8-diaza-spiro[4.5]decan-2-one; (352) 8-(2-[1.3]-Dioxan-2-yl-ethyl)-4-(4-fluorobenzyl)-3-(4-isobutoxybenzyl)-1-oxa-3,8-diaza-spiro[4.5]decane-3-one; (353) 4-(4-Fluorobenzyl)-3-(4-isobutoxybenzyl)-8-{3-[(S)-4-isopropyl-2-oxo-oxazolidin-3-yl]-propyl}-1-oxa-3,8-diaza-spiro[4.5]decane-3-one; (354) N-{1-[2-(1,3-Dioxolan-2-yl)ethyl]piperidin-4-yl}-N-(4-fluorobenzyl)-W-(4-isobutoxybenzyl)carbamide hydrochloride; (355) N-{1-[2-(1,3-Dioxan-2-yl)ethyl]-piperidin-4-yl}-N-(4-fluorobenzyl)-2-[4-(2-hydroxy-2-methylpropoxy)phenyl]-acetamide tartrate; (356) N-(4-Fluorobenyzl)-N-(piperidin-4-yl)-2-(4-isobutoxyphenyl)acetamide; (357) N-{1-[3-(3,5-Dimethylpiperidin-1-yl)propyl]piperidin-4-yl-}-N-(4-fluorobenzyl)-2-(4-isobutoxyphenyl)acetamide dihydrochloride; (358) 1-[3-(4-{(4-Fluorobenzyl)-[2-(4isobutoxyphenyl)acetyl]amino}piperidin-1-yl)propyl]piperidine-4-carboxylic acid methyl ester dihydrochloride; (359) N-(4-Fluorobenzyl)-2-(4-isobutoxyphenyl)-N-{1-[2-(1-methylpyrrolidin-2-yl-)ethyl]piperidin-4-yl}acetamide dioxalate; (360) N-{1-[3-(2,6-Dimethylmorpholin-4-yl)propyl]piperidin-4-yl}-N-(4-fluorobenzyl)-2-(4-isobutoxyphenyl)acetamide dioxalate; (361) N-(4-Fluorobenzyl)-N-{1-[3-(3-hydroxypiperidin-1-yl)propyl]piperidin-4-yl}-2-(4-isobutoxyphenyl)acetamide dioxalate; (362) N-(4-Fluorobenzyl)-2-(4-isobutoxyphenyl)-N-{1-[3-(2-methylpiperidin-1-yl)-propyl]piperidin-4-yl}acetamide dioxalate; (363) N-(4-Fluorobenzyl-2-(4-isobutoxyphenyl)-N-[1-(3-pyrrolidin-1-yl-propyl)piperidin-4-yl]acetamide dioxalate; (364) N-{1-[3-(2,5-Dimethylpyrrolidin-1-yl)propyl]piperidin-4-yl}-N-(4-fluorobenzyl)-2-(4-isobutoxyphenyl)acetamide dioxalate; (365) N-(4-Fluorobenzyl)-N-{1-[3-(3-hydroxyrnethylpiperidin-1-yl)propyl]piperidin-4-yl}-2-(4-isobutoxyphenyl)acetamide dioxalate; (366) N-(4-Fluorobenzyl)-2-(4-isobutoxyphenyl)-N-{1-[3-(4-(S)-isopropyl-2-oxo-oxazolidin-3-yl)propyl]piperidin-4-yl}acetamide oxalate; (367) N-[2-(4-Fluorophenyl)ethyl]-2-(4-isobutoxyphenyl)-N-{1-[3-(4-(S)-isopropyl-2-oxo-oxazolidin-3-yl)propyl]piperidin-4-yl}acetamide oxalate; (368) N42-(4-Fluorophenyl)ethyl]-N-{1-[3-(4-(S)-isopropyl-2-oxo-oxazolidin-3-yl)propyl]piperidin-4-yl}-2-(4-propoxyphenyl)acetamide oxalate; (369) N-(4-Fluorobenzyl)-N-{1-[3-(4-(S)-isopropyl-2-oxo-oxazolidin-3-yl)propyl]piperidin-4-yl}-2-(4-propoxyphenyl)acetamide oxalate; (370) N-{1-[2-(1,3-Dioxan-2-yl)ethyl]piperidin-4-yl}-N-(4-fluorobenzyl)-2-(4-isobutoxyphenyl)acetamide oxalate; (371) N-{1-[2-(1,3-Dioxan-2-yl)ethyl]piperidin-4-yl}-N-[2-(4-fluorophenyl)ethyl]-2-(4-isobutoxyphenyl)acetamide oxalate; (372) N-{1-[2-(1,3-Dioxan-2-yl)ethyl]piperidin-4-yl}-N-[2-(4-fluorophenyl)ethyl]-2-(4-propoxyphenyl)acetamide oxalate; (373) N-{1-[2-(1,3-Dioxan-2-yl-)ethyl]piperidin-4-yl}-N-(4-fluorobenzyl)-2-(4-propoxyphenyl)acetamide tartrate; (374) N-{1-[2-(1,3-Dioxan-2-yl)ethyl]piperidin-4-yl}-N-(4-fluorobenzyl)-W-(4-isobutoxybenzyl)carbarnide tartrate; (375) N-{1-[2-(1,3-Dioxan-2-yl)ethyl]piperidin-4-yl}-N-(4-fluorobenzyl)-2-(4-fluorophenyl)acetamide tartrate; (376) N-{1-[2-(1,3-Dioxan-2-yl)ethyl]piperidin-4-yl}-N-(4-fluorobenzyl)-2-p-tolylacetamide tartrate; (377) 2-Benzofuran-5-yl-N-{1-[2-(1,3-dioxan-2-yl)ethyl]piperidin-4-yl}-N-(4-fluorobenzyl)acetamide tartrate; (378) 2-(2,3-Dihydrobenzofuran-5-yl)-N-{1-[2-(1,3-dioxan-2-yl)ethyl]piperidin-4-yl}-N-(4-fluorobenzyl)acetamide tartrate; (379) N-{1-[2-(2,2-Dimethyl-1,3-dioxolan-4-yl)ethyl]piperidin-4-yl}-N-(4-fluorobenzyl)-2-(4-isobutoxyphenyl)acetamide tartrate; (380) N-{1-[2-(1,3-Dioxan-4-yl)ethyl]piperidin-4-yl}-N-(4-fluorobenzyl)amine; (381) N-{1-[2-(1,3-Dioxan-4-yl)ethyl]piperidin-4-yl}-N-(4-fluorobenzyl)-2-(4-isobutoxyphenyl)acetamide tartrate; (382) N-{1-[2-(1,3-Dioxan-4-yl)ethyl]piperidin-4-yl}-N(4-fluorobenzyl)-2-(4-trifluorornethylphenyl)acetamide tartrate; (383) 2-(4-Cyanophenyl)-N-{1-[2-(1,3-dioxan-4-yl)ethyl]piperidin-4-yl}-N-(4-fluorobenzyl)acetamide tartrate; (384) N-(4-Fluorobenzyl)-2-(4-isobutoxyphenyl)-N-{1-[2-(2-oxo-imidazolidin-1-yl)ethyl]piperidin-4-yl}acetamide hydrochloride; (385) 2-(4-Methoxyphenyl)-N-(4-methylbenzyl)-N-{1-[2-(2-oxo-imidazolidin-1-yl)ethyl]piperidin-4-yl}acetamide hydrochloride; (386) N-(4-Fluorobenzyl)-2-(4-isopropoxyphenyl)-N-{1-[2-(2-oxo-imidazolidin-1-yl)ethyl]piperidin-4-yl}acetamide hydrochloride; (387) N-(4-Fluorobenzyl)-2-(4-isopropoxyphenyl)-N-{1-[3-(3-methyl-2-oxo-2,3-dihydro-benzoimidazol-1-yl)propyl]piperidin-4-yl}acetamide hydrochloride; (388) N-{1-[2-(2,4-Dioxo-1,4-dihydro-2H-quinazolin-3-yl)ethyl]piperidin-4-yl}-2-(4-methoxyphenyl)-N-(4-methylbenzyl)acetamide hydrochloride; (389) 2-(4-Methoxyphenyl)-N-(4-methylbenzyl)-N-{1-[3-(2-oxo-2,3-dihydrobenzoimidazol-1-yl)propyl]piperidin-4-yl}-acetamide hydrochloride; (390) N-(4-Fluorobenzyl)-2-(4-isopropoxyphenyl)-N-{1-[4-(2-oxo-2,3-dihydrobenzoimidazol-1-yl)butyl]piperidin-4-yl}acetamide hydrochloride; (391) N-{1-[2-(2,4-Dioxo-1,4-dihydro-2H-quinazolin-3-ypethyl]piperidin-4-yl}-N-(4-fluorobenzyl)-2-(4-isopropoxyphenyl)acetamide hydrochloride; (392) 4-(4-Fluorobenzylamino)-piperidine-1-carboxylic acid benzyl ester; (393) N-(1-Benzyloxycarbonylpiperidin-4-yl)-N-(4-fluorobenzyl)-N′-(4-isopropoxybenzyl)carbamide; (394) N-(4-Fluorobenzyl)-N′-(4-isopropoxybenzyl)-N-piperidin-4-yl-carbamide oxalate; (395) N-{1-[2-(1,3-Dioxolan-2-yl)ethyl]piperidin-4-yl}-N-(4-fluorobenzyl)-W-(4-isopropoxy-benzyl)carbamide oxalate; (396) N-{1-[2-(1,3-Dioxolan-2-yl)ethyl]piperidin-4-yl]2-(4-methoxyphenyl)-N-(4-methylbenzyl)acetamide hydrochloride; (397) N-{1-[2-(1,3-Dioxolan-2-yl-)ethyl]piperidin-4-yl}-N-(4-fluorobenzyl)-2-(4-isobutoxyphenyl)acetamide hydrochloride; (398) N-{1-[2-(1,3-Dioxolan-2-yl)ethyl]piperidin-4-yl}-2-(4-isopropoxyphenyl)-N-(4-methylbenzyl)acetamide hydrochloride; (399) N-{1-[2-(1,3-Dioxolan-2-yl)ethyl]piperidin-4-yl}-N-(4-fluorobenzyl)-2-(4-propoxyphenyl)acetamide tartrate; (400) N-(4-Fluorobenzyl)-N′-(4-isopropoxybenzyl)-N-{1-[2-((S)-4-methyl-1,3-dioxolane-2-yl)ethyl]piperidin-4-yl}carbamide oxalate; (401) N-(4-Fluorobenzyl)-N′-(4-isopropoxybenzyl)-N-[1-(3-morpholin-4-yl-propyl)piperidin-4-yl]carbamide oxalate; (402) 2-(4-Methoxyphenyl)-N-(4-methylbenzyl)-N-[1-(2-morpholin-4-yl-ethyl)piperidin-4-yl]acetamide dihydrochloride; (403) 2-(4-Methoxyphenyl)-N-(4-methylbenzyl)-N-[1-(3-morpholin-4-ylpropyl)piperidin-4-yl]acetamide dihydrochloride; (404) N-(4-Fluorobenzyl)-2-(4-isobutoxyphenyl)-N-[1-(3-morpholin-4-ylpropyl)piperidin-4-yl]acetamide dihydrochloride; (405) N-(4-Fluorobenzyl)-2-(4-isopropoxy-phenyl)-N-[1-(3-morpholin-4-yl-propyl)piperidin-4-yl]acetamide dihydrochloride; (406) N-(4-Fluorobenzyl)-N′-(4-isopropoxybenzyl)-N-[1-(3-piperidin-1-yl-propyl)piperidin-4-yl]carbamide oxalate; (407) N-(4-Fluorobenzyl)-N′-(4-isopropoxybenzyl)-N-[1-(3-((S)-4-isopropyl-2-oxazolidinon-1-yl-propyl)piperidin-4-yl]carbamide tartrate; (408) N-(4-Fluorobenzyl)-N′-(4-isopropoxybenzyl)-N-{1-[2-(2,5,5-trimethyl-1,3-dioxan-2-yl)ethyl]}piperidin-4-yl]carbamide oxalate; (409) N-{1-[3-(1,3-Dioxolan-2-yl)propyl]piperidin-4-yl}-N-(4-fluorobenzyl)-N′-(4-isopropoxybenzyl)carbamide oxalate; (410) N-[1-(2,2-Dimethyl-1,3-dioxan-5-yl)-piperidin-4-yl]-N-(4-fluorobenzyl)-N′-(4-isopropoxybenzyl)carbamide oxalate; (411) N-(4-Fluorobenzyl)-N′-(4-isopropoxybenzyl)-N-{[2-(1-methyl pyrrolidin-2-yl)ethyl]-piperidin-4-yl}carbamide oxalate; (412) N-[1-(2,2-Dimethyl-1,3-dioxan-5-yl)piperidin-4-yl]-N-(4-fluorobenzyl)-2-(4-isobutoxyphenyl)acetamide oxalate; (413) N-[1-(1,3-Dioxan-5-yl)-piperidin-4-yl)-N-(4-fluorobenzyl)-2-(4-isobutoxyphenyl)acetamide tartrate; (414) N-[1-(2,2-Dimethyl-1,3-dioxan-5-yl)piperidin-4-yl]-N-(4-fluorobenzyl)-2-(4-fluorophenyl)acetamide tartrate; (415) N-{1-[2-(1,3-Dioxan-4-yl)ethyl]piperidin-4-yl}-N-(4-fluorobenzyl)-2-(4-fluorophenyl)acetamide tartrate: (416) N-{1-[2-(1,3-Dioxan-4-yl)ethyl]piperidin-4-yl}-N-(4-fluorobenzyl)-2-(4-trifluoromethoxyphenyl)acetamide tartrate: (417) N-{1-[2-(1,3-Dioxan-4-yl)ethyl]piperidin-4-yl}-N-(4-fluorobenzyl)-2-(4-propoxyphenyl)acetamide tartrate; (418) N-(4-Fluorobenzyl)-2-(4-isobutoxyphenyl)-N-[1-(tetrahydropyran-4-yl)piperidin-4-yl]acetamide tartrate; (419) N-(4-Fluorobenzyl)-2-(4-isobutoxyphenyl)-N-[1-(tetrahydropyran-4-ylmethyl)piperidin-4-yl]acetamide tartrate; (420) N-(4-Fluorobenzyl)-2-(4-isobutoxyphenyl)-N-{1-[2-(tetrahydropyran-4-yl)ethyl]piperidin-4-yl]acetamide tartrate; (421) N-(4-Fluorobenzyl)-2-(4-fluorophenyl)-N-[1-(tetrahydropyran-4-yl)piperidin-4-yl]acetamide tartrate; (422) N-[1-((S)-3,5-Dihydroxypentyl)piperidine-4-yl]-N-(4-fluorobenzyl)-2-(4-isobutoxyphenyl)acetamide tartrate; (423) N-{1-[2-((4S)-1,3-Dioxane-4-yl)ethyl]piperidine-4-yl}-N-(4-fluorobenzyl)-2(4-isobutoxyphenyl)acetamide tartrate; (424) N-{1-[2-(1,3-Dioxan-2-yl)ethyl]piperidin-4-yl}-N-(4-fluorobenzyl)amine; (425) 2-(4-Benzyloxyphenyl)-N-{1-[2-(1,3-dioxan-2-yl)ethyl]piperidin-4-yl}-N-(4-fluorobenzyl)acetamide tartrate; (426) N-{1-[2-(1,3-Dioxan-2-yl)ethyl]piperidin-4-yl}-N-(4-fluorobenzyl)-2-(4-hydroxyphenyl)-acetamide tartrate; (427) N-{1-[2-(1,3-Dioxan-2-yl)ethyl]piperidin-4-yl}-N-(4-fluorobenzyl)-2-(4-rnethoxyphenyl)-acetamide tartrate; (428) N-{1-[2-(1,3-Dioxan-2-yl)ethyl]piperidin-4-yl}-N-(4-fluorobenzyl)-2-(4-isopropylphenyl)-acetamide tartrate; (429) N-{1-[2-(1,3-Dioxan-2-yl)ethyl]piperidin-4-yl}-N-(4-fluorobenzyl)-2-(4-trifluorornethoxy-phenyl)acetamide tartrate; (430) N-{1-[2-(1,3-Dioxan-2-yl)ethyl]-piperidin-4-yl}-N-(4-fluorobenzyl)-2-(4-ethoxyphenyl)-acetamide oxalate; (431) N-{1-[2-(1,3-Dioxan-2-yl)ethyl]piperidin-4-yl}-N-(4-fluorobenzyl)-2-(4-isopropoxyphenyl)-acetamide oxalate; (432) N-{1-[2-(1,3-Dioxan-2-yl)ethyl]piperidin-4-yl}-N-(4-fluorobenzyl)-2-phenylacetamide oxalate; (433) N-{1-[2-(1,3-Dioxan-2-yl)ethyl]piperidin-4-yl}-N-(4-fluorobenzyl)-2-[4-(2-fluoroethoxy)-phenyl]acetamide oxalate; (434) N-{1-[2-(5,5-Dimethyl-1,3-dioxan-2-yl)ethyl]piperidin-4-yl}-N-(4-fluorobenzyl)-2-(4-isobutoxyphenyl)acetamide oxalate; (435) N-(4-Fluorobenzyl)-2-(4-isobutoxyphenyl)-N-{1-[2-((R)-4-methyl-1,3-dioxan-2-yl)ethyl]-piperidin-4-yl}acetamide oxalate; (436) N-(4-Fluorobenzyl)-2-(4-isobutoxyphenyl)-N-{1-[2-((S)-4-methyl-1,3-dioxolan-2-yl)ethyl]piperidin-4-yl}acetamide oxalate; (437) N-{1-[2-(4,6-Dimethyl-1,3-dioxan-2-yl)ethyl]piperidin-4-yl}-N-(4-fluorobenzyl)-2-(4-isobutoxyphenyl)acetamide oxalate; (438) N-(4-Fluorobenzyl)-N-{1-[24(S)-4-methyl-1,3-dioxolan-2-yl)ethyl]piperidin-4-yl}-2-(4-trifluoromethoxyphenyl)acetamide oxalate; (439) N-(4-Fluorobenzyl)-2-(4-isopropylphenyl)-N-{1-[2-((S)-4-methyl-1,3-dioxolan-2-yl)ethyl]-piperidin-4-yl}acetamide oxalate; (440) N-(4-Fluorobenzyl)-N-{1-[2-((R)-4-rnethyl-1,3-dioxan-2-yl)ethyl]piperidin-4-yl}-2-(4-trifluorornethoxyphenyl)acetamide oxalate; (441) N-(4-Fluorobenzyl)-2-(4-isobutoxyphenyl)-N-{1-[2-(2,5,5-trimethyl-1,3-dioxan-2-yl)ethyl]piperidin-4-yl}acetamide oxalate; (442) N-(4-Fluorobenzyl)-2-(4-isobutoxyphenyl)-N-{1-[2-(2-methyl-1,3-dioxolan-2-yl)ethyl]-piperidin-4-yl-}acetamide oxalate; (443) N-(4-Fluorobenzyl)-2-(4-isobutoxyphenyl)-N-{1-[3-(1,3-dioxolan-2-yl)propyl]piperidin-4-yl}acetamide tartrate; (444) N-(4-Fluorobenzyl)-2-(4-isobutoxyphenyl)-N-{1-(3-piperidin-1-yl-propyl)piperidin-4-yl}-acetamide dihydrochloride; (445) N-(4-Fluorobenzyl)-2-(4-isobutoxyphenyl)-N-{1-[2-(tetrahydropyran-2-yloxy)ethyl]-piperidin-4-yl}acetamide oxalate; (446) N-(4-Fluorobenzyl)-2-(4-isobutoxyphenyl)-N-{1-[3-(2-oxo-piperidin-1-yl)propyl]piperidin-4-yl}acetamide; (447) N-(4-Fluorobenzyl)-2-(4-isobutoxyphenyl)-N-{1-[3-(2-oxo-pyrrolidin-1-yl)propyl]piperidin-4-yl}acetamide hydrochloride; (448) N-(4-Fluorobenzyl)-2-(4-isobutoxyphenyl)-N-{1-[3-((R)-4-isopropyl-2-oxo-oxazolidin-3-yl)propyl]piperidin-4-yl}acetamide oxalate; (449) N-(4-Fluorobenzyl)-2-(4-isobutoxyphenyl)-N-{1-[3-(2-oxo-oxazolidin-3-yl)propyl]piperidin-4-yl}acetamide oxalate; (450) N-(4-Fluorobenzyl)-2-(4-isobutoxyphenyl)-N-{1-[3-((S)-4-methyl 2-oxo-oxazolidin-3-yl)propyl]piperidin-4-yl}acetamide tartrate; (451) N-(4-Fluorobenzyl)-2-(4-isobutoxyphenyl)-N-{1-[3-((S)-4-ethyl-2-oxo-oxazolidin-3-yl)-propyl]piperidin-4-yl}acetamide oxalate; (452) N-(4-Fluorobenzyl)-2-(4-isobutoxyphenyl)-N-{1-[2-(1,3-oxothiolan-2-yl)ethyl]piperidin-4-yl}acetamide L-tartrate; (453) 2-(4-Bromophenyl)-N-{1-[2-(1,3-dioxan-2-yl)ethyl)piperidin-4-yl}-N-(4-fluorobenzyl)-acetamide L-tartrate; (454) N-{1-[2-(1,3-Dioxan-2-yl)ethyl)piperidin-4-yl}-N-(4-fluorobenzyl)-2-(4-isobutylamino-phenyl)acetamide L-tartrate; (455) N-{1-[2-(1,3-Dioxan-2-yl)ethyl)piperidin-4-yl}-N-(4-fluorobenzyl)-2-(4-propylaminophenyl)acetamide L-tartrate; (456) N-{1-[2-(1,3-Dioxan-2-yl)ethyl)piperidin-4-yl}-N-(4-fluorobenzyl)-2-(4-(1-nitropropyl)-phenyl)acetamide L-tartrate; (457) N-{1-[2 (1,3-Dioxan-2-yl)ethyl)piperidin-4-yl}-N-(4-fluorobenzyl)-2-[4-(2-oxopyrrolidin-1-yl)phenyl)acetamide L-tartrate; (458) N-{1-[2-(1,3-Dioxan-2-yl)ethyl)piperidin-4-yl}-N-(4-fluorobenzyl)-2-(4-isobutylsulfanylphenyl)acetamide L-tartrate; (459) N-{1-[2-(1,3-Dioxan-2-yl)ethyl)piperidin-4-yl}-N-(4-fluorobenzyl)-2-(4-iodophenyl)-acetamide L-tartrate; (460) 2-(4-Acetophenyl)-N-{1-[2-(1,3-dioxan-2-yl)ethyl)piperidin-4-yl}-N-(4-fluorobenzyl)-acetamide L-tartrate; (461) 2-[4-(1-hydroxyiminoethyl)phenyl]-N-{1-[2-(1,3-dioxan-2-yl)ethyl)piperidin-4-yl}-N-(4-fluorobenzyl)acetamide L-tartrate; (462) N-{1-[2-(1,3-Dioxan-2-yl)ethyl)piperidin-4-yl}-N-(4-fluorobenzyl)-2-(4-morpholin-4-yl-phenyl)acetamide L-tartrate; (463) N-{1-[2-(1,3-Dioxan-2-yl)ethylypiperidin-4-yl}-N-(4-fluorobenzyl)-2-(4-pyrazol-1-ylphenyl)acetamide L-tartrate; (464) N-{1-[2-(1,3-Dioxan-2-yl)-1-methylethyl]piperidin-4-yl}-N-(4-fluorobenzyl)-2-(4-isobutoxyphenyl)-acetamide L-tartrate; (465) N-{1-[2-(1,3-Dioxan-4-yl)ethyl)piperidin-4-yl}-N-(4-fluorobenzyl)-2-(4-pyrazol-1-ylphenyl)acetamide L-tartrate; (466) N-[1-((R)-3,5-Dihydroxypentyl)pipe-ridine-4-yl]-N-(4-fluorobenzyl)-2-(4-isobutoxyphenyl)acetamide tartrate; (467) N-{1-[2-((4R)-1,3-Dioxane-4-yl)ethyl]piperidine-4-yl}-N-(4-fluorobenzyl)-2-(4-isobutoxyphenyl)acetamide tartrate; (468) N-{1-[2-(1,3-Dioxan-2-yl)ethyl]piperidin-4-yl}-N-(4-fluorobenzyl)-2-[4-(1,2,4-triazol-4-yl)phenyl]acetamide L-tartrate; (469) nortriptyline; (470) duloxetine; (471) lofepramine; (472) tomoxetine; (473) 3-({1-[2-(7-methyl-5-oxo-5H)-[1,3]thiazolo[3,2-a]pyrimidin-6-yl)ethyl]-3-pyrrolidinyl}methyl)-1H-indole-5-carbonitrile hydrochloride; (474) 3-({1-[2-(6-chloro-2-oxo-2,3-dihydro-1H-indol-5-yl)ethyl]-3-pyrrolidinyl}-methyl)-1H-indole-5-carbonitrile hydrochloride; (475) moclobemide; (476) N-acetylserotonin; (477) bromfaromine; (478) beflaxozone; (479) chlorimipramine; (480) cyanimipramine; (481) cianopramine; (482) desipramine; (483) protriptyline; (484) trimipramine; (485) doxepin; (486) cyclobenzaprine; (487) 5-methoxycarbonylamino-N-acetyltryptamine; (488) amoxapine; (489) maprotiline; (490) fefazodone; (491) flesinoxan hydrochloride; (492) urapidil; (493) WY47846 (3a,4,4a,6a,7,7a-hexahydro-2-[4-[4-(2-pyrimidinyl)-1-piperazinyl]-butyl]-4,7-etheno-1H-cyclobutano[f]isoindole-1,3(2H)-dione dihydrochloride sesquihydrate); (494) SM3997 (N-[4-[4-(2-pyrimidinyl)-1-piperazinyl]butyl]-bicyclo[2.2.1]heptane-2,3-di-exo-carboximide); (495) 2-(4-(4-(2-pyrimidinyl)-1-piperazinyl-propyl)-1,2-benzoisothiazol-3-(2H)-one 1,1-dioxide hydrochloride; (496) KC9172 (3-butyl-9,9-dimethyl-7-[4-[4-[2-methoxyphenyl)-1-piperazinyl]butyl]-3,7-diazabicyclo[3,2,1]nonane-2,4,6,8-tetraone); (497) 4-(N,N-dipropylamino)-6-methoxy-1,3,4,5-tetrahydrobenz-[c,d]indole; (498) 4-[4-(N-1,2-benzisothiazol-3(2H)-one 1,1-dioxido)]butylamino-6-methoxy-1,3,4,5-tetrahydrobenz[c,d]-indole hydrochloride; (499) 5-carboxamidotryptamine; (500) N,N-dipropyl-5-carboxamidotryptamine; (501) AH25086 (3-(2-aminoethyl)-1H-indole-5-(N-methyl)acetamide); (502) GR43175 (3-(2-dimethylaminoethyl)-1H-indole-5-(N-methyl)methanesulfonamide); (503) 3-(2-[4-[2-(1,2-benzisothiazole-3(2H)-one 1,1-dioxido)]butyl]amino)ethyl-5-methoxy-1H-indole; (504) spiroxatrine; (505) MDL72832 (8-[4-(1,4-benzodioxan-2-ylmethylamino)butyl]-8-azaspiro-[4,5]decane-7,9-dione); (506) 2-[4-(1,4-benzodioxan-2-ylmethylamino)butyl]-1,2-benzisothiazol-3(2H)-one 1,1-dioxide; (507) 2-(N,N-dipropylamino)-8-hydroxy-1,2,3,4-tetrahydronaphthalene; (508) 2-{4-[2-(1,2-benzisothiazol-3(2H)-one 1,1-dioxido)]butyl}amino-8-methoxy-1,2,3,4-tetrahydronaphthalene; (509) 3-N,N-dipropylamino-5-hydroxy-thiochroman; 3-N,N-dipropylamino-5-ethoxy-thiochroman; (510) 3-N,N-dipropylamino-5-ethoxychroman; (511) 1-[2-(3-indolyl)]-ethyl-2,6-dimethylpiperidine; (512) 1-{2-[3-(5-carboxamido)indolyl]}ethyl-2,6-dimethylpiperidine; (513) RU24924 (5-methoxy-3-(1,2,3,6-tetrahydropyridin-4-yl]-1H-indole); (514) 5-methoxy-3-(1,2,3,6-tetrahydropyridin-5-yl)-1H-indole; (515) diethyl N-benzyloxycarbonyl-5-benzyloxycarbonyloxy-L-tryptophyl-L-aspartate; (516) dibenzyl N-benzyloxycarbonyl-5-hydroxy-L-tryptophanylaspartate; (517) 5-Hydroxy-L-tryptophyl-L-aspartic acid trihydrate; (518) diethyl N-benzyloxycarbonyl-5-hydroxy-L-tryptophyl-L-glutamate; (519) diethyl 5-hydroxy-L-tryptophyl-L-glutamate hydrochloride; (520) dibenzyl L-benzyloxycarbonyl-5-hydroxytryptophyl-L-glutamate; (521) 5-hydroxy-L-tryptophyl-L-glutamic acid; (522) pentachlorophenyl ester of N-benzyloxycarbonyl-5-hydroxy-L-tryptophan; (523) methyl ester of N-benzyloxycarbonyl-5-hydroxy-L-tryptophyl-L-tyrosine; (524) N-Acetyl-5-hydroxy-L-tryptophan; (525) methyl ester of N-acetyl-5-hydroxy-L-tryptophyl-L-tyrosine; (526) methyl ester of N-acetyl-5-hydroxy-L-tryptophyl-5-hydroxy-L-tryptophan; (527) 5-hydroxy-L-tryptophyl-L-alanine hydrate; (528) 5-hydroxy-L-tryptophan-L-valine; (529) 5-hydroxy-L-tryptophyl-L-leucine; (530) 5-hydroxy-L-tryptophyl-L-proline; (531) 5-hydroxy-L-tryptophyl-L-phenylalanine; (532) 5-hydroxy-L-tryptophyl-5-hydroxy-L-tryptophan; (533) 5-hydroxy-L-tryptophyl-L-tryptophan; (534) 1-(5-hydroxy)tryptophyl-L-serine; (535) 5-hydroxy-L-tryptophyl-L-arginine; (536) 5-hydroxy-L-tryptophylglycine; (537) 5-hydroxy-1-tryptophyl-gamma-aminobutyric acid; (538) 5-hydroxy-L-tryptophanamide hydrate; (539) methyl ester of 5-hydroxy-L-tryptophyl-L-histidine; (540) benzyl ester of L-5-hydroxytryptophan; (541) benzyl ester of N-benzyloxycarbonyl-5-hydroxy-L-tryptophyl-5-hydroxy-L-tryptophan; (542) 5-Hydroxy-L-tryptophyl-5-hydroxy-L-tryptophan hemihydrate; (543) 5-hydroxytryptophan inosinate; (544) theophylline salt of (DL) 5-hydroxytryptophan; (545) RU25591 (6,7,8,9-tetrahydro N, N-dimethyl 5-[4-nitrophenyl]oxy 5H-benzocyclohepten 7-amine) cis-fumarate); (546) LM5008 (4-[2-(3-indolyl)ethyl]piperidine); (547) DU24565 (6-nitro-2-(1-piperazinyl)quinoline); (548) CGP6085A (4-(5,6-dimethyl-2-benzofuranyl) piperidine hydrochloride); (549) alaprociate; (550) dibenzoxazepine; (551) deprenyl; (552) isocarboxazide; (553) furazolidone; (554) procarbazine; (555) Ro 60-0175/ORG 35030 ((S)-2-(4,4,7-trimethyl-1,4-dihydro-indeno (1,2-B) pyrrol-1-yl)-1-methyl-ethylamine) (556) Ro 60-0332/ORG 35035 ((S)-2-(Chloro-5-fluoro-indol-1-yl)-1-methylethylamine); (557) 1-[6-Chloro-5-trifluoromethyl)-2-pyridinyl]-piperazine hydrochloride; (558) 5-carboxyamidotryptamine; (559) SB 206553 (3,5-Dihydro-5-methyl-N-3-pyridinylbenzo[1,2-b:4,5-bldipyrrole-1(2H)-carboxamide hydrochloride); (560) ondansetron; (561) granisetron; (562) tropisetron; (563) dolasetron; (564) palonosetron; (565) trimethobenzamide; (566) risperidone; (567) clozapine; (568) azatadine; (569) cyproheptadine; (570) fenclonine; (571) chlorpromazine; (572) (313)-2,3-dihydrolysergine; (573) (313)-2,3-dihydroisolysergine; (574) (3β, 5β, 8β)-9,10-didehydro-2,3-dihydro-6-methylergoline-8-acetonitrile; (575) 25I-NBMD (2-(4-iodo-2,5-dimethoxyphenyl)-N-[(2,3-methylenedioxyphenyl)methyl]ethanamine); (576) N-(2-methoxybenzyl)-1-(8-bromo-2,3,6,7-tetrahydrobenzo[1,2-b:4,5-b′]difuran-4-yl)-2-aminoethane; (577) 5-benzyloxytryptamine; (578) 5-methoxy-7-N,N-dimethyltryptamine; (579) A372159 ((11S,16R)-3-[4-(propan-2-yloxy)-2-(trifluoromethyl)phenyl]-6-oxa-10,14-diazatetracyclo[8.6.1.0^{5,17}.0^{11,16}]heptadeca-1,3,5(17)-triene); (580) AL-34662 (14(S)-2-Aminopropyl)-1H-indazol-6-ol); (581) AL-37350A ((S)-(+)-1-(2-Aminopropyl)-8,9-dihydropyrano[3,2-e]indole); (582) AL-38022A ((S)-2-(8,9-dihydro-7H-pyrano[2,3-g]indazol-1-yl)-1-methylethylamine); (583) AS-19 ((25)-N,N-dimethyl-5-(1,3,5-trimethylpyrazol-4-yl)-1,2,3,4-tetrahydronaphthalen-2-amine); (584) alnespirone; (585) BIMU8 (N-[(1R,5S)-8-methyl-8-azabicyclo[3.2.1]oct-3-yl]-2-oxo-3-(propan-2-yl)-2,3-dihydro-1H-benzimidazole-1-carboxamide hydrochloride); (586) BMY-14802 (1-(4-fluorophenyl)-4-[4-(5-fluoropyrimidin-2-yl)piperazin-1-yl]butan-1-01); (587) BRL-54443 (3-(1-methylpiperidin-4-yl)-1H-indol-5-ol); (588) batoprazine; (589) benzylpiperazine; (590) binospirone; (591) 1-(8-bromobenzo[1,2-b;4,5-b]difuran-4-yl)-2-aminopropane); (592) CP-809,101 (2-[(3-Chlorophenyl)methoxy]-6-(1-piperazinyl)pyrazine); (593) CP-93,129 (3-(1,2,3,6-tetrahydropyrid in-4-yl)-1,4-d ihydropyrrolo[3,2-b]pyridin-5-one); (594) CP-94,253 (3-(1,2,5,6-tetrahydro-4-pyridyl)-5-propoxypyrrolo[3,2-b]pyridine); (595) CGS-12066A (4-(4-methylpiperazin-1-yl)-7-(trifluoromethyl)pyrrolo[1,2-a]quinoxaline); (596) chlorophenylbiguanide; (597) chlorphentermine; (598) dazopride; (599) dimemebfe; (600) 2,5-dimethoxy-4-bromoamphetamine; (601) 2,5-dimethoxy-4-fluoroamphetamine; (602) 2,5-dimethoxy-4-methylamphetamine; (603) EMD-386,088 (5-chloro-2-methyl-3-(1,2,3,6-tetrahydro-4-pyridinyl)-1H-indole); (604) EMDT (2-(2-ethyl-5-methoxy-1-indol-3-yl)-N,N-dimethylethanamine); (605) p-fluoropiperazine; (606) fluprazine; (607) jimscaline; (608) LY-293,284 ((4R)-6-acetyl-4-(di-n-propylamino)-1,3,4,5-tetrahydrobenz[c,d]indole); (609) lasmitidan; (610) lorcaserin; (611) 2-methyl-5-hydroxytryptamine; (612) 2-methyl-4,5-methylenedioxyamphetamine; (613) NBUMP (N-[4-[4-(2-methoxyphenyl)piperazin-1-yl]butyl]adamantane-1-carboxamide); (614) 1-(1-naphthyl)piperazine; (615) Org-37,684 ((3S)-3-[(2,3-dihydro-5-methoxy-1H-inden-4-yl)oxy]pyrrolidine); (616) PNU-22394 (6-Methyl-1,2,3,4,5,6-hexahydro-azepino[4,5-b]indole)); (617) PRX-00023 (N-(3-[4-(4-cyclohexylmethanesulfonylaminobutyl)piperazin-1-yl]phenyl)acetamide); (618) RH-34 (3-[2-(2-methoxybenzylamino)ethyl]-1H-quinazoline-2,4-dione); (619) RS56812 (N-[(3R)-1-azabicyclo[2.2.2]oct-3-yl]-2-(1-methyl-1H-indol-3-yl)-2-oxoacetamide); (620) RS67333 (1-(4-amino-5-chloro-2-methoxyphenyl)-3-(1-butyl-4-piperidinyl)-1-propanone); (621) RU24969 (5-Methoxy-3-(1,2,5,6-tetrahydro-4-pyridinyl)-1H-indole); (622) Ro60-0175 ((S)-6-Chloro-5-fluoro-1H-indole-2-propanamine); (623) TFMFIy ((2R)-1-(8-trifluoromethyl-2,3,6,7-tetrahydrobenzo[1,2-b:4,5-Mdifuran-4-yl)-2-aminoethane); (624) U92016-A ((8R)-8-(Dipropylamino)-6,7,8,9-tetrahydro-3H-benz[e]indole-2-carbonitrile) (625) VER3323 ((2S)-1-(6-bromo-2,3-dihydroindol-1-yl)propan-2-amine); (626) vilazodone; (627) WAY-181,187 (1-[(2S,5S)-4,4-difluoro-5-(hydroxymethyl)tetrahydrofuran-2-yl]pyrimidine-2,4(1H,3H)-dione); (628) WAY-208,466 (N′-[(2Z)-4-(2,4-dichlorophenyl)-3-(2-methylpropyl)-1,3-thiazol-2(3H)-ylidene]-2-(pyrazin-2-yloxy)acetohydrazide); (629) YM-348 (2S)-1-(7-ethyl-1H-furo[2,3-Mindazol-1-yl)propan-2-amine); (630) alprenolol; (631) BMY 7378 (8-(2-[4-(2-methoxyphenyl)-1-piperazinyl]ethyl)-8-azaspiro[4.5]decane-7,9-dione); (632) cyanopindolol; (633) iodocyanopindolol; (634) lezcotozan; (635) methiothepin; (636) NAN-190 (1-(2-methoxyphenyl)-4-(4-phthalimidobutyl)piperazine); (637) oxprenolol; (638) pindolol; (639) propranolol; (640) robalzotan; (641) S15535 (1-(2,3-dihydro-1,4-benzodioxin-8-yl)-4-(2,3-dihydro-1H-inden-2-yl)piperazine); (642) spiperone; (643) TFMPP; (644) UH-301 ((S)-5-fluoro-8-hydroxy-2-(dipropylamino)tetralin); (645) WAY-100,135 ((S)—N-tert-butyl-3-(4-(2-methoxyphenyl)-piperazin-1-yl)-2-phenylpropanamide); (646) WAY-100,635 (N-[2-[4-(2-methoxyphenyl)-1-piperazinyl]ethyl]-N-(2-pyridyl)cyclohexanecarboxamide); (647) mefway; (648) 5-hydroxytryptophan; (649) 5-hydroxytryptophan creatinine sulfate complex; (650) 5-methoxytryptamine; (651) 5-methoxytryptamine creatinine sulfate complex; (652) 5-HIAA (5-hydroxyindoleacetic acid); and (653) 5-HIAA (5-hydroxyindoleacetic acid) creatinine sulfate complex; and the salts, solvates, analogues, congeners, bioisosteres, hydrolysis products, metabolites, precursors, and prodrugs thereof.

35. The method of claim 21 wherein the composition comprises metformin or a salt thereof and melatonin or a salt thereof.

36. The method of claim 35 wherein the composition comprises metformin hydrochloride and melatonin.

37. The method of claim 21 wherein the composition consists essentially of the first and second agents.

38. The method of claim 21 wherein the composition further comprises a pharmaceutically acceptable carrier.

39. The method of claim 21 wherein the first agent is associated with a carrier substance to transport the first agent to an intended site of action.

40. The method of claim 21 wherein the second agent is associated with a carrier substance to transport the second agent to an intended site of action.

41. The method of claim 21 wherein the composition comprises the first agent and the second agent in a weight ratio of 1-1000:0.01-1.

42. The method of claim 21 wherein the composition comprises the first agent and the second agent in a weight ratio of 1-100:0.05-1.

43. The method of claim 21 wherein the composition comprises the first agent and the second agent in a weight ratio of 10-100:0.1-1.

44. The method of claim 21 wherein the composition is administered parenterally, orally, nasally, rectally, topically or buccally.

45. The method of claim 44 wherein parenteral administration is a route of administration selected from the group consisting of subcutaneous, intracutaneous, intravenous, intramuscular, intraarticular, intraarterial, intrasynovial, intrasternal, intrathecal, intralesional, and intracranial injection.


SEQUENCE LISTING (7 sequences)

SEQ ID NO: 1 - protein, 9 aa, Homo sapiens
SAFSVGLET

SEQ ID NO: 2 - protein, 6 aa, Homo sapiens
PIRFTK

SEQ ID NO: 3 - protein, 16 aa, Homo sapiens
FYNQQNHYDGSTGKFH

SEQ ID NO: 4 - protein, 11 aa, Homo sapiens
NIPGLYYFAYH

SEQ ID NO: 5 - protein, 17 aa, Homo sapiens
TYVMKDVKVSLFKKDKA

SEQ ID NO: 6 - protein, 22 aa, Homo sapiens
TYDQYQENNVDGASGSVLLHLE

SEQ ID NO: 7 - protein, 13 aa, Homo sapiens
GDNVYLNVYGEGE